(19) United States
(12) Patent Application Publication
(10) Pub. No.: US 2011/0190161 A1
(43) Pub. Date: Aug. 4, 2011

(54) Methylation Profile of Cancer

(75) Inventors: Anatoliy A. Melnikov, Glenview, IL (US); Victor V. Levenson, Chicago, IL (US)

(73) Assignee: NORTHWESTERN UNIVERSITY, Evanston, IL (US)

(21) Appl. No.: 13/085,067

(22) Filed: Apr. 12, 2011

Related U.S. Application Data
(63) Continuation of application No. 11/872,956, filed on Oct. 16, 2007.
(60) Provisional application No. 60/852,360, filed on Oct. 17, 2006.

(51) Int. Cl. C40B 30/04; C12Q 1/68

(52) U.S. Cl. 506/9; 435/6.11

(57) ABSTRACT

The present invention relates to compositions and methods for cancer diagnostics, including but not limited to, cancer markers. In particular, the present invention provides methods of identifying methylation patterns in genes associated with specific cancers.

### CROSS-REFERENCE TO RELATED APPLICATIONS

The present application claims the benefit under 35 U.S.C. §119(e) to U.S. provisional application No. 60/852,360, filed on Oct. 17, 2007, the content of which is incorporated herein by reference in its entirety.

### BACKGROUND

The present invention relates to compositions and methods for cancer diagnostics, including but not limited to, cancer markers. In particular, the present invention provides methods of identifying methylation patterns in genes associated with specific cancers.

Early detection of cancer can save lives and the importance of early detection of cancer can hardly be underestimated. Early diagnosis has profound effects on survival rate, quality of life, and overall cost to society, so screening for cancer provides a valuable opportunity to promote a shift in stage distribution to earlier stages and to increased survival.

For example, for breast cancer, radiological screening techniques (mammography, ultrasonography, computed tomography, magnetic resonance imaging) have contributed greatly to early detection. Unfortunately, detection rates of mammography depend on tissue density (up to 100% sensitivity in fatty versus 47%—in dense breasts) and the stage of the disease (81% for invasive ductal carcinomas (IDC) versus 55% for ductal carcinomas in situ, DCIS). Increased sensitivity (up to 89% for DCIS) comes with magnetic resonance imaging, which can be enhanced even further by a combination of different techniques. Unfortunately, the cost of these procedures for screening is unacceptably high and results can vary from one observer to another.

Thus, there is a need in the art for reliable diagnostic (e.g., detection) and prognostic methods to identify and monitor cancer (e.g., breast, ovarian, pancreatic, liver, colon, etc.) that do not depend on tissue density or experience of the observer.

### SUMMARY

The present invention relates to compositions and methods for cancer diagnostics, including but not limited to, cancer markers. In particular, the present invention provides methods of identifying methylation patterns in genes associated with specific cancers.

Accordingly, in some embodiments, the present invention provides a method, comprising providing a biological sample from a subject (e.g., blood, bodily fluid, tissue, cytological sample), the biological sample comprising genomic DNA; detecting the presence or absence of DNA methylation in one or more genes to generate a methylation profile for the subject; and comparing the methylation profile to one or more standard methylation profiles, wherein the standard methylation profiles are selected from the group consisting of methylation profiles of non-cancerous samples and methylation profiles of cancerous samples. In certain embodiments, the detecting the presence or absence of DNA methylation comprises the digestion of the genomic DNA with a methylation-sensitive restriction enzyme followed by amplification of gene-specific DNA fragments, which optionally may include multiplex amplification. Optionally, the amplified DNA may include one or more CpG sequences or CpG islands which are not digested by the methylation-sensitive restriction enzyme.

In further embodiments, the present invention provides a method of characterizing cancer, comprising providing a biological sample from a subject diagnosed with cancer, the biological sample comprising genomic DNA; and detecting the presence or absence of DNA methylation in one or more genes or one or more sets of genes (e.g., each set containing 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 52, 53, 54, 55, 56, . . . genes), examples of which are listed in Table 1, thereby characterizing cancer in the subject. In some embodiments, the methylation status of the promoter region of the gene is investigated. In some embodiments, the characterization of cancer comprises detecting the presence or absence of chemotherapy resistant cancer.

TABLE 1

| | | Alternative | | |
|---|---|---|---|---|
| Gene | HUGO name | symbol | Alternative name | Genbank # |
| ABCB1 | ATP binding cassette, sub- | MDR1 | multidrug resistance 1 | X58723 |
| | family B, member 1 | | | |
| ACTB | actin beta | | beta actin | Y00474 |
| APAF1 | apoptotic peptidase activating | | apoptotic protease | AC013283 |
| | factor | | activating factor | |
| BRCA1 | breast cancer 1, early onset | BRCA | breast and ovarian cancer | U37574 |
| | | | susceptibility protein 1 | |
| CALCA | calcitonin/calcitonin-related | CALC | calcitonin | X15943 |
| | polypeptide, alpha | | | |
| CASP8 | caspase 8, apoptotis-rclatcd | | caspase 8 | AB038980 |
| | cysteine peptidase | | | |
| CCND2 | cyclin D2 | CYC D2 | | U47284 |
| CDHI | cadherin 1 | | E-cadherin | L34545 |
| CDKN1A | cyclin-dependent kinase | p21waf1/cip1, | | AF497972 |
| | inhibitor 1A | p21 | | |
| CDKN1B | cycin-dependent kinase | p27kip1 | | AB005590 |
| | inhibitor 1B | | | |
| CDKN1C | cyclin-dependent kinase | p57kip2, p57 | | D64137 |
| | inhibitor 1C | | | |
| CDKN2A | cyclin-dependent kinase | p16INK4A | | NT_037734 |
| | inhibitor 2A | | | |
| CDKN2B | cyclin-dependent kinase | p15INK4B, p15 | | NT_037734 |
| | inhibitor 2B | | | |
| DAPK I | death associated protein | DAPK | death associated protein | AL161787 |
| | kinase 1 | | kinase | |
| DNAJC15 | dnaJ (Hsp40) homolog, | MCJ | methylation controlled J | NT_024524 |
| | subfamily C, member 15 | | protein | |
| EDNRB | endothelin receptor type B | | | AF114163 |
| EP300 | E1A binding protein p300 | | | AL080243 |
| ESR1 promoter A | estrogen receptor 1 | ERaA | estrogen receptor alpha | AL356311 |
| | | | (proximal) | |
| ESR1 promoter B | estrogen receptor 1 | ERaB | estrogen receptor alpha | |
| | | | (distal) | |
| FABP3 | fatty acid binding protein 3 | MDGI | mammary derived growth | U17081 |
| | | | inhibitor | |
| FAS | Fas (TNF receptor | CD95 | | X87625 |
| | superfamily, member 6) | | | |
| FHIT | fragile histidine triad gene | | | AF399855 |
| GPC3 | glypican 3 | | | AF003529 |
| GSTP1 | glutathione-S-transferase p1 | GSTP | | M37065 |
| HIC1 | hypermethylated in cancer 1 | HIC | | L41919 |
| ICAM1 | intercellular adhesion | CD54 | | M65001 |
| | molecule 1 | | | |
| MCTS1 | malignant T cell amplified | MCT-1 | | AC011890 |
| | sequence | | | |
| MGMT | O-6-methylguanine DNA | | | X61657 |
| | methyltransferase | | | |
| MLH1 | mutL homolog 1 | HMLH1 | | AC011816 |
| MSH2 | mutS homolog 2 | hMSH2 | | AB006445 |
| MUC2 | mucin 2, intestinal/tracheal | | mucin 2 | U67167 |
| MYOD1 | myogenic differentiation 1 | MYF3 | myogenic factor 3 | AC124056 |
| NR3C1 | nuclear receptor subfamily 3, | GR | glucocorticoid receptor | M69074 |
| | group C, member 1 | | | |
| PAX5 | paired box gene 5 | | | AF268279 |
| PGK1 | phosphoglycerate kinase 1 | PGK | | M34017 |
| PGR distal | progesterone receptor | PR, PR-2D | progesterone receptor | X51730 |
| | | | distal promoter | |
| PGR proximal | progesterone receptor | PR, PR-1A | progesterone receptor | X51730 |
| | | | proximal promoter | |
| PLAU | plasminogen activator, | uPA | urokinase plasminogen | X02419 |
| | urokinase | | activator | |
| PRDM2 | PR domain containing 2, with | RIZ 1, RIZ | retinoblastoma protein- | AF472587 |
| | ZNF domain | | interacting zinc finger | |
| | | | protein | |
| PRKCDBP | protein kinase C, delta binding | SRBC | serum deprivation | AF408198 |
| | protein | | response factor (sdr)- | |
| | | | related gene product that | |
| | | | binds to c-kinase | |
| PYCARD | PYD and CARD domain | TMS1 | target of methylation- | AF184072 |
| | containing | | induced silencing-1 | |
| RARB | retinoic acid receptor, beta | RAR beta 2, | retinoic acid receptor | X56849 |
| | | RARB2, RAR | beta 2 | |
| RASSF1 | Ras associated (RalGDS/ | RASSF1A | | AC002481 |
| | AF-6) domain family 1 | | | |
| RB I | retinoblastoma 1 | | | AL392048 |
| RPL15 | ribosoinal protein L15 | | | AB061823 |
| S100A2 | S100 calcium binding protein | S100+ | | AL162258 |
| | A2 | | | |
| SCGB3A1 | secretoglobin, family 3A, | HIN1 | high in normal-1 | AC006207 |
| | member 1 | | | |
| SFN | stratifin | | 14-3-3 sigma | AF029081 |
| SLC19A1 | solute carrier family 19 (folate | RFC1, RFC | reduced folate carrier | U92868 |
| | transporter), member 1 | | | |
| SOCS1 | suppressor of cytokine | SOCS | | Z46940 |
| | signaling 1 | | | |
| SYK | spleen tyrosine kinase | | | AC021581 |
| TES | testis derived transcript | | | A1250865 |
| THBS1 | thrombospondin 1 | THBS | | J04835 |
| TNFSF11 | tumor necrosis factor (ligand) | TRANCE, | osteoprotegerin ligand | AF333234 |
| | superfamily, member 11 | TRANKL, | | |
| | | OPGL | | |
| TP73 | tumor protein p73 | p73 | | AF235000 |
| VHL | von Hippel-Lindau tumor | | | AF010238 |
| | suppressor | | | |

In other embodiments, the characterization of cancer comprises determining a chance (quantitative or qualitative) of disease-free survival. In still further embodiments, the characterization of cancer comprises determining the risk of developing metastatic disease. In yet other embodiments, the characterization of cancer comprises monitoring disease progression in a subject. In some embodiments, the biological sample is a biopsy sample. In other embodiments, the biological sample is a blood plasma sample. In further embodiments, the biological sample is a cytological sample that has been fixed (e.g., with a fixative or preservative such as Preservcyt® Solution). In some embodiments, the DNA methylation may comprise CpG methylation. In some preferred embodiments, detecting the presence or absence of DNA methylation comprises the digestion of said genomic DNA with a methylation-sensitive restriction enzyme followed by amplification of gene-specific DNA fragments, which optionally may be a multiplex amplification. In some embodiments, the methylation-sensitive restriction enzyme comprises Hin6I. In other embodiments the methylation sensitive restriction enzyme comprises HpaII. In certain embodiments, the cancer is breast, ovarian, colon, pancreatic, liver, lung and/or prostatic.

The present invention further provides a method of diagnosing cancer, comprising providing a biological sample from a subject, the biological sample comprising genomic DNA; and detecting the presence or absence of DNA methylation in one or more genes listed in Table 1, thereby diagnosing cancer in the subject. In some embodiments, the subject is at high risk of developing cancer.

The present invention additionally provides a kit for characterizing cancer, comprising reagents for (e.g., sufficient for) detecting the presence or absence of DNA methylation in one or more genes listed in Table 1. In some embodiments, the kit further comprises instructions for using the kit for characterizing cancer in the subject. In some embodiments, the instructions comprise instructions required by the United States Food and Drug Administration for use in in vitro diagnostic products. In some embodiments, the reagents comprise reagents for digestion of genomic DNA comprising the one or more genes with a methylation-sensitive restriction enzyme followed by amplification of gene-specific DNA fragments (optionally multiplex amplification of DNA fragments having CpG methylation). In some embodiments, characterizing cancer comprises detecting the presence or absence of chemotherapy resistant cancer. In other embodiments, characterizing cancer comprises determining a chance of disease-free survival. In still further embodiments, characterizing cancer comprises determining the risk of developing metastatic disease. In yet other embodiments, characterizing cancer comprises monitoring disease progression in the subject.

In some embodiments, the present invention provides a method of characterizing or detecting cancer, comprising providing a biological sample from a subject suspected of having cancer or diagnosed with cancer, the biological sample comprising genomic DNA; and detecting the presence or absence of DNA methylation in one or more of the genes listed in Table 1, thereby characterizing or diagnosing cancer in the subject.

In one embodiment, the subject is suspected of having ovarian cancer. In some embodiments, the biological sample tested from a subject suspected of having ovarian cancer is tested for the presence or absence of DNA methylation in one or more of the following genes; FHIT, MLH1, DNAJC15, FAS, MGMT, progesterone receptor (PGR), RARB, RPL15, PYCARD, PLAU and S100A2.

In one embodiment, the subject is suspected of having prostate cancer. In some embodiments, the biological sample tested from a subject suspected of having prostate cancer is tested for the presence or absence of DNA methylation in one or more of the following genes; BRCA1, CALCA, CASP8, CYCD2, EDNRB, EP300, FHIT, GPC3, NR3C1, HIC1, DNAJC15, FABP3, ABCB1, MSH2, CDKN1A, CDKN1C, PAX5, PGK1, progesterone receptor (“PGR” which may include the proximal promoter “PR-1P” or the distal promoter “PR-2D”), S100A2, TES, THBS and VHL.

In one embodiment, the subject is suspected of having lung cancer. In some embodiments, the biological sample tested from a subject suspected of having lung cancer is tested for the presence or absence of DNA methylation in one or more of the following genes; CASP8, CDKN1C, VHL, PAX5, DAPK1, NR3C1, MGMT, progesterone receptor PGR proximal or distal promoter (e.g., PR-1P or PR-2D), MLH1, SLC19A1, TES, TNFSF11, CYCD2, MYOD1, RB1, SFN, ESR1 promoter A or promoter B, and GPC3.

In one embodiment, the subject is suspected of having pancreatic cancer. In some embodiments, the biological sample tested from a subject suspected of having pancreatic cancer is tested for the presence or absence of DNA methylation in one or more of the following genes; SFN, BRCA1, DAPK1, EDNRB, NR3C1, DNAJC15, MUC2, CDKN1A, CDKN1C, PGK1, progesterone receptor (e.g., PR-1P or PR-2D), S100A2, TES and VHL.

In one embodiment, the subject is suspected of having colon cancer. In some embodiments, the biological sample tested from a subject suspected of having colon cancer is tested for the presence or absence of DNA methylation in one or more of the following genes; BRCA1, CASP8, CYCD2, DAPK1, ERAB, GPC3, NR3C1, ABCB1, MYOD1, CDKN1A, CDKN1C, PGK1, progesterone receptor PGR proximal or distal promoter (e.g., PR-1P or PR-2D), RAR, RB1, SLC19A1, RPL15, S100A2, SOCS1, TES, THBS and VHL.

In some embodiments, the methods may be used to diagnose or characterize cancer or hyperplasia in a subject (e.g., ovarian cancer, lung cancer, prostate cancer, pancreatic cancer, colon cancer, invasive ductal carcinoma (IDC) of breast tissue, ductal carcinoma in situ (DCIS) of breast tissue, atypical ductal hyperplasia (ADH) of breast tissue, or combinations thereof). The methods may include: (a) reacting isolated genomic DNA from the subject and a methylation-sensitive restriction enzyme; wherein the genomic DNA comprises a plurality of promoters from different genes, and the enzyme cleaves unmethylated promoters and does not cleave methylated promoters; (b) contacting the genomic DNA thus reacted and a plurality of pairs of specific primers in an amplification mixture (optionally a multiplex amplification mixture), the pairs of specific primers being configured to hybridize to the genomic DNA and to amplify a plurality of different promoters through a region comprising an uncleaved promoter; (c) reacting the amplification mixture; (d) detecting one or more amplified promoters in the reacted amplification mixture or the absence thereof, thereby diagnosing or characterizing cancer or hyperplasia in the subject. Optionally, a promoter may include a CpG sequence which is methylated or unmethylated (e.g., a CpG sequence within a CpG island). Diagnosing or characterizing may include diagnosing or characterizing therapy resistant forms of cancer or hyperplasia (e.g., chemotherapy resistant forms of cancer or hyperplasia).

In the methods, genomic DNA may be isolated from any suitable biological sample from the subject. In some embodiments, genomic DNA is isolated from blood, plasma, or serum. In other embodiments, genomic DNA is isolated from tissue.

In the methods, the amplified promoters in a reacted amplification mixture may be detected by any suitable means. In some embodiments, one or more amplified promoters in the reacted amplification mixture are detected (or their absence is detected) by: (1) contacting a microarray and the reacted amplification mixture, the microarray comprising a plurality of DNA samples, each of which hybridizes to one of the plurality of different promoters; and (2) detecting hybridization or the lack of hybridization between DNA in the reacted amplification mixture and one or more of the plurality of DNA samples of the microarray thereby obtaining a methylation profile. In further embodiments, the methylation profile of the subject may be compared to a standard methylation profile (e.g., a standard methylation profile for non-cancerous samples, a standard methylation profile for cancerous samples, or both).

The methods may utilize control samples. In some embodiments, the) methods include: (a) separating isolated genomic DNA from the subject into: (i) a control sample and (ii) an experimental sample; and (b) adding control nucleic acid to both the control and experimental samples, wherein the control nucleic acid comprises at least one known promoter that is unmethylated (e.g., within a CpG sequence). In further embodiments, the control sample may not be reacted with the methylation-sensitive restriction enzyme and the experimental sample may be reacted with the methylation-sensitive restriction enzyme, where both the control and experimental samples are contacted with primers for the control nucleic acid under conditions such that a fragment of the control nucleic acid is amplified if the known promoter is uncleaved. Control samples may include control DNA comprising promoters for one or more control genes (e.g., ACTB, GADPH, and TUBA3 genes).

The methods typically utilize a plurality of pairs of specific primers. In some embodiments, the plurality of pairs of specific primers comprises at least five (5) pairs of specific primers (or at least ten (10) pairs of specific primers). The plurality of pairs of specific primers may be configured to amplify one or more genes as disclosed herein in order to diagnose cancer or hyperplasia in a subject.

The methods may include diagnosing cancer in a subject (e.g., pancreatic cancer or colon cancer) by: (a) reacting a plasma sample from the subject and reagents for detecting methylation status of genomic DNA in the sample; and (b) determining the methylation status for a plurality of genes to generate a methylation profile, thereby diagnosing cancer in the subject. Reagents for detecting methylation status may include one or more of the following: methylation-sensitive restriction enzymes; bisulfite reagents for converting unmethylated cytosine to uracil; and specific oligonucleotides that may be used as probes or as primers in an amplification mixture (and optionally may be designed to hybridize to methylated or unmethylated cytosine residues either before or after treatment with bisulfite).

The disclosed methods may include diagnosing hyperplasia in breast tissue of a subject. In some embodiments of the methods, each of the five pairs of specific primers is configured to amplify a gene selected from the group consisting of EP300, MGMT, TP73, PGR (distal promoter), THBS1, PYCARD (TMS1), PRKCDBP (SRBC), FABP3 (MDG1), MSH2, HIC1, BRCA1, TES, NR3C1 (GR), ICAM1, DAPK1, TNFSF11 (RANKL), DNAJC15 (MCJ), CDH1, CASP8, RPL15, and PGK1.

The disclosed methods may exhibit high sensitivity, high selectivity, or both high sensitivity and high selectivity in diagnosing cancer or hyperplasia. In some embodiments, the methods exhibit sensitivity of at least about 80% (preferably 85%, 90%, 95%, or 99%). In some embodiments, the methods exhibit selectivity of at least about 80% (preferably 85%, 90%, or 95%).

### BRIEF DESCRIPTION OF THE FIGURES

FIG. 1 shows the differences in methylated genes between normal blood and blood from subjects with ovarian cancer.

FIG. 2 shows the differences in methylated genes between normal blood and blood from subjects with lung cancer.

FIG. 3 shows the results of the methylation assay of the present invention applied to normal blood compared to blood from a subject with prostate cancer.

FIG. 4 shows the CpG methylation profile of genes from the blood of normal subjects when compared to that of blood from pancreatic cancer patients.

FIG. 5 shows methylation profiling in blood from normal subjects compared to that of patients with colon cancer.

FIG. 6 provides a general schema of the M³ assay. Isolated DNA is divided into two aliquots, and one of them is incubated with Hin6I, while the other is left untreated. Both are used for PCR amplification with gene-specific primers, the products are labeled with different fluorophores, mixed and used for competitive hybridization with the array. After signal processing and statistical analysis selected diagnostic gene set is evaluated in all specimens.

FIG. 7 provides the layout for genes present on a microarray. The microarray contains 64 positions (8×8 format) with 3 empty and 61 occupied spots. Three spots (ACTS*, GAPDH*, and TUBA3*) contain probes for transcribed sequences of corresponding genes, while another spot is occupied by a probe for genomic DNA of A. thaliana. One of the remaining probes (HTLF) is defective. Accordingly, 61 occupied spots contain four controls and one defective probe, leaving 56 spots for analysis. Two promoters are evaluated for ESR1 (A and B) and PGR (proximal and distal).

FIG. 8 provides a graphic representation of performance of the M³-assay with heterogeneous samples. Genomic DNA from MCF7 and T47D was mixed at different ratio and used for analysis. Methylation status of MYOD1, PAX5, RPL15, and RB1 was determined as described and plotted against the percentage of unmethylated genes. Cy5/Cy3 ratio remains at the level of SMC for all genes with no less than 50% of methylated fragments, and such genes are scored as methylated. Further increase in Cy5/Cy3 ratio reflects prevalence of unmethylated fragments in the sample.

### DETAILED DESCRIPTION

To facilitate an understanding of the present invention, a number of terms and phrases are defined below:

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

As used herein, the term “subject suspected of having cancer” refers to a subject that presents one or more symptoms indicative of a cancer (e.g., a noticeable lump or mass). A subject suspected of having cancer may also have one or more risk factors. A subject suspected of having cancer has generally not been tested for cancer. However, a “subject suspected of having cancer” encompasses an individual who has received an initial diagnosis (e.g., a CT scan showing a mass) but for whom the sub-type or stage of cancer is not known. The term further includes people who once had cancer (e.g., an individual in remission).

As used herein, the term “subject at risk for cancer” refers to a subject with one or more risk factors for developing a specific cancer. Risk factors include, but are not limited to, genetic predisposition, environmental expose, preexisting non-cancer disease, and lifestyle.

As used herein, the term “stage of cancer” refers to a numerical measurement of the level of advancement of a cancer. Criteria used to determine the stage of a cancer include, but are not limited to, the size of the tumor, whether the tumor has spread to other parts of the body and where the cancer has spread (e.g., within the same organ or region of the body or to another organ).

As used herein, the term “providing a prognosis” refers to providing information regarding the impact of the presence of cancer (e.g., as determined by the diagnostic methods of the present invention) on a subject's future health (e.g., expected morbidity or mortality).

As used herein, the term “subject diagnosed with a cancer” refers to a subject having cancerous cells. The cancer may be diagnosed using any suitable method, including but not limited to, the diagnostic methods of the present invention.

As used herein, the term “instructions for using said kit for detecting cancer in said subject” includes instructions for using the reagents contained in the kit for the detection and characterization of cancer in a sample from a subject. In some embodiments, the instructions further comprise the statement of intended use required by the U.S. Food and Drug Administration (FDA) in labeling in vitro diagnostic products.

As used herein, the term “detecting the presence or absence of DNA methylation” refers to the detection of DNA methylation in the promoter region of one or more genes (e.g., cancer markers of the present invention) of a genomic DNA sample. The detecting may be carried out using any suitable method, including, but not limited to, those disclosed herein.

As used herein, the term “detecting the presence or absence of chemotherapy resistant cancer” refers to detecting a DNA methylation pattern characteristic of a tumor that is likely to be resistant to chemotherapeutic agents (e.g., selective estrogen receptor modulators (SERMs)).

As used herein, the term “determining a chance of disease-free survival” refers to the determining the likelihood of a subject diagnosed with cancer surviving without the recurrence of cancer (e.g., metastatic cancer). In some embodiments, determining a chance of disease free survival comprises determining the DNA methylation pattern of the subject's genomic DNA.

As used herein, the term “determining the risk of developing metastatic disease” refers to likelihood of a subject diagnosed with cancer developing metastatic cancer. In some embodiments, determining the risk of developing metastatic disease comprises determining the DNA methylation pattern of the subject's genomic DNA.

As used herein, the term “monitoring disease progression in said subject” refers to the monitoring of any aspect of disease progression, including, but not limited to, the spread of cancer, the metastasis of cancer, and the development of a pre-cancerous lesion into cancer. In some embodiments, monitoring disease progression comprises determining the DNA methylation pattern of the subject's genomic DNA.

As used herein, the term “methylation profile” refers to a presentation of methylation status of one or more cancer marker genes in a subject's genomic DNA. In some embodiments, the methylation profile is compared to a standard methylation profile comprising a methylation profile from a known type of sample (e.g., cancerous or non-cancerous samples or samples from different stages of cancer). In some embodiments, methylation profiles are generated using the methods of the present invention. The profile may be presented as a graphical representation (e.g., on paper or on a computer screen), a physical representation (e.g., a gel or array) or a digital representation stored in computer memory.

As used herein, the term “non-human animals” refers to all non-human animals. Such non-human animals include, but are not limited to, vertebrates such as rodents, non-human primates, ovines, bovines, ruminants, lagomorphs, porcines, caprines, equines, canines, felines, ayes, etc.

The term “gene” refers to a nucleic acid (e.g., DNA) sequence that comprises coding sequences necessary for the production of a polypeptide, precursor, or RNA (e.g., rRNA, tRNA). The polypeptide can be encoded by a full length coding sequence or by any portion of the coding sequence so long as the desired activity or functional properties (e.g., enzymatic activity, ligand binding, signal transduction, immunogenicity, etc.) of the full-length or fragment are retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

In addition to containing introns, genomic forms of a gene may also include sequences located on both the 5′ and 3′ end of the sequences that are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, post-transcriptional cleavage and polyadenylation.

The term “wild-type” refers to a gene or gene product that has the characteristics of that gene or gene product when isolated from a naturally occurring source. A wild-type gene is that which is most frequently observed in a population and is thus arbitrarily designed the “normal” or “wild-type” form of the gene. In contrast, the term “modified” or “mutant” refers to a gene or gene product that displays modifications in sequence and or functional properties (i.e., altered characteristics) when compared to the wild-type gene or gene product. It is noted that naturally-occurring mutants can be isolated; these are identified by the fact that they have altered characteristics when compared to the wild-type gene or gene product.

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

DNA molecules are said to have “5′ ends” and “3′ ends” because mononucleotides are reacted to make oligonucleotides or polynucleotides in a manner such that the 5′ phosphate of one mononucleotide pentose ring is attached to the 3′ oxygen of its neighbor in one direction via a phosphodiester linkage. Therefore, an end of an oligonucleotide or polynucleotide is referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a nucleic acid sequence, even if internal to a larger oligonucleotide or polynucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand. The promoter and enhancer elements that direct transcription of a linked gene are generally located 5′ or upstream of the coding region. However, enhancer elements can exert their effect even when located 3′ of the promoter element or the coding region. Transcription termination and polyadenylation signals are located 3′ or downstream of the coding region.

Transcriptional control signals in eukaryotes comprise “promoter” and “enhancer” elements. Promoters and enhancers consist of short arrays of DNA sequences that interact specifically with cellular proteins involved in transcription (T. Maniatis et al., Science 236:1237 (1987)). Promoter and enhancer elements have been isolated from a variety of eukaryotic sources including genes in yeast, insect and mammalian cells, and viruses (analogous control elements, i.e., promoters, are also found in prokaryote). The selection of a particular promoter and enhancer depends on what cell type is to be used to express the protein of interest. Some eukaryotic promoters and enhancers have a broad host range while others are functional in a limited subset of cell types (for review see, Voss et al., Trends Biochem. Sci., 11:287 (1986); and T. Maniatis et al., supra). For example, the SV40 early gene enhancer is very active in a wide variety of cell types from many mammalian species and has been widely used for the expression of proteins in mammalian cells (Dijkema et al., EMBO J. 4:761 (1985)). Two other examples of promoter/enhancer elements active in a broad range of mammalian cell types are those from the human elongation factor la gene (Uetsuki et al., J. Biol. Chem., 264:5791 (1989); Kim et al., Gene 91:217 (1990); and Mizushima and Nagata, Nuc. Acids. Res., 18:5322 (1990)) and the long terminal repeats of the Rous sarcoma virus (Gorman et al., Proc. Natl. Acad. Sci. USA 79:6777 (1982)) and the human cytomegalovirus (Boshart et al., Cell 41:521 (1985)). Some promoter elements serve to direct gene expression in a tissue-specific manner.

As used herein, the term “promoter/enhancer” denotes a segment of DNA which contains sequences capable of providing both promoter and enhancer functions (i.e., the functions provided by a promoter element and an enhancer element, see above for a discussion of these functions). For example, the long terminal repeats of retroviruses contain both promoter and enhancer functions. The enhancer/promoter may be “endogenous” or “exogenous” or “heterologous.” An “endogenous” enhancer/promoter is one that is naturally linked with a given gene in the genome. An “exogenous” or “heterologous” enhancer/promoter is one that is placed in juxtaposition to a gene by means of genetic manipulation (i.e., molecular biological techniques such as cloning and recombination) such that transcription of that gene is directed by the linked enhancer/promoter.

As used herein, the terms “complementary” or “complementarity” are used in reference to polynucleotides (i.e., a sequence of nucleotides) related by the base-pairing rules. For example, for the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

The term “homology” refers to a degree of complementarity. There may be partial homology or complete homology (i.e., identity). A partially complementary sequence is a nucleic acid molecule that at least partially inhibits a completely complementary nucleic acid molecule from hybridizing to a target nucleic acid is “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous nucleic acid molecule to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target that is substantially non-complementary (e.g., less than about 30% identity); in the absence of non-specific binding the probe will not hybridize to the second non-complementary target.

When used in reference to a double-stranded nucleic acid sequence such as a cDNA or genomic clone, the term “substantially homologous” refers to any probe that can hybridize to either or both strands of the double-stranded nucleic acid sequence under conditions of low stringency as described above.

When used in reference to a single-stranded nucleic acid sequence, the term “substantially homologous” refers to any probe that can hybridize (i.e., it is the complement of) the single-stranded nucleic acid sequence under conditions of low stringency as described above.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids. A single molecule that contains pairing of complementary nucleic acids within its structure is said to be “self-hybridized.”

As used herein, the term “Tₘ” is used in reference to the “melting temperature.” The melting temperature is the temperature at which a population of double-stranded nucleic acid molecules becomes half dissociated into single strands. The equation for calculating the Tₘ of nucleic acids is well known in the art. As indicated by standard references, a simple estimate of the Tₘ value may be calculated by the equation: Tₘ=81.5+0.41(% G+C), when a nucleic acid is in aqueous solution at 1 M NaCl (See e.g., Anderson and Young, Quantitative Filter Hybridization, in Nucleic Acid Hybridization (1985)). Other references include more sophisticated computations that take structural as well as sequence characteristics into account for the calculation of Tₘ.

As used herein the term “stringency” is used in reference to the conditions of temperature, ionic strength, and the presence of other compounds such as organic solvents, under which nucleic acid hybridizations are conducted. With “high stringency” conditions, nucleic acid base pairing will occur only between nucleic acid fragments that have a high frequency of complementary base sequences. Thus, conditions of “weak” or “low” stringency are often required with nucleic acids that are derived from organisms that are genetically diverse, as the frequency of complementary sequences is usually less.

“High stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄.H₂O and 1.85 g/l EDTA, pH adjusted to 7.4 with NaOH), 0.5% SDS, 5×Denhardt's reagent and 100 μg/ml denatured salmon sperm DNA followed by washing in a solution comprising 0.1×SSPE, 1.0% SDS at 42° C. when a probe of about 500 nucleotides in length is employed.

“Medium stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄.H₂O and 1.85 g/l EDTA, pH adjusted to 7.4 with NaOH), 0.5% SDS, 5×Denhardt's reagent and 100 μg/ml denatured salmon sperm DNA followed by washing in a solution comprising 1.0×SSPE, 1.0% SDS at 42° C. when a probe of about 500 nucleotides in length is employed.

“Low stringency conditions” comprise conditions equivalent to binding or hybridization at 42° C. in a solution consisting of 5×SSPE (43.8 g/l NaCl, 6.9 g/l NaH₂PO₄.H₂O and 1.85 g/l EDTA, pH adjusted to 7.4 with NaOH), 0.1% SDS, 5×Denhardt's reagent (50×Denhardt's contains per 500 ml: 5 g Ficoll (Type 400, Pharamcia), 5 g BSA (Fraction V; Sigma)) and 100 μg/ml denatured salmon sperm DNA followed by washing in a solution comprising 5×SSPE, 0.1% SDS at 42° C. when a probe of about 500 nucleotides in length is employed.

The art knows well that numerous equivalent conditions may be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of low stringency hybridization different from, but equivalent to, the above listed conditions. In addition, the art knows conditions that promote hybridization under conditions of high stringency (e.g., increasing the temperature of the hybridization and/or wash steps, the use of formamide in the hybridization solution, etc.) (see definition above for “stringency”).

“Amplification” is a special case of nucleic acid replication involving template specificity. It is to be contrasted with non-specific template replication (i.e., replication that is template-dependent but not dependent on a specific template). Template specificity is here distinguished from fidelity of replication (i.e., synthesis of the proper polynucleotide sequence) and nucleotide (ribo- or deoxyribo-) specificity. Template specificity is frequently described in terms of “target” specificity. Target sequences are “targets” in the sense that they are thought to be sorted out from other nucleic acid. Amplification techniques have been designed primarily for this sorting out.

Template specificity is achieved in most amplification techniques by the choice of enzyme. Taq and Pfu polymerases, by virtue of their ability to function at high temperature, are found to display high specificity for the sequences bounded and thus defined by the primers; the high temperature results in thermodynamic conditions that favor primer hybridization with the target sequences and not hybridization with non-target sequences (H. A. Erlich (ed.), PCR Technology, Stockton Press (1989)).

As used herein, the term “amplifiable nucleic acid” is used in reference to nucleic acids that may be amplified by any amplification method. It is contemplated that “amplifiable nucleic acid” will usually comprise “sample template.”

As used herein, the term “sample template” refers to nucleic acid originating from a sample that is analyzed for the presence of “target”. In contrast, “background template” is used in reference to nucleic acid other than sample template that may or may not be present in a sample. Background template is most often inadvertent. It may be the result of carryover, or it may be due to the presence of nucleic acid contaminants thought to be purified away from the sample. For example, nucleic acids from organisms other than those to be detected may be present as background in a test sample.

As used herein, the term “primer” refers to an oligonucleotide, whether occurring naturally as in a purified restriction digest or produced synthetically, that is capable of acting as a point of initiation of synthesis when placed under conditions in which synthesis of a primer extension product that is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer and the use of the method.

As used herein, the term “probe” refers to an oligonucleotide (i.e., a sequence of nucleotides), whether occurring naturally as in a purified restriction digest or produced synthetically, recombinantly or by PCR amplification, that is capable of hybridizing to another oligonucleotide of interest. A probe may be single-stranded or double-stranded. Probes are useful in the detection, identification and isolation of particular gene sequences. It is contemplated that any probe used in the present invention will be labeled with any “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

As used herein, the term “polymerase chain reaction” (“PCR”) refers to the method of K. B. Mullis U.S. Pat. Nos. 4,683,195, 4,683,202, and 4,965,188, hereby incorporated by reference, which describe a method for increasing the concentration of a segment of a target sequence in a mixture of genomic DNA without cloning or purification. This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing and polymerase extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified”.

With PCR, it is possible to amplify a single copy of a specific target sequence in genomic DNA to a level detectable by several different methodologies (e.g., hybridization with a labeled probe; incorporation of biotinylated primers followed by avidin-enzyme conjugate detection; incorporation of ³²P-labeled deoxynucleotide triphosphates, such as dCTP or dATP, into the amplified segment). In addition to genomic DNA, any oligonucleotide or polynucleotide sequence can be amplified with the appropriate set of primer molecules. In particular, the amplified segments created by the PCR process are, themselves, efficient templates for subsequent PCR amplifications. As used herein, the terms “PCR product,” “PCR fragment,” and “amplification product” refer to the resultant mixture of compounds after two or more cycles of the PCR steps of denaturation, annealing and extension are complete. These terms encompass the case where there has been amplification of one or more segments of one or more target sequences.

As used herein, the term “amplification reagents” refers to those reagents (deoxyribonucleotide triphosphates, buffer, etc.), needed for amplification except for primers, nucleic acid template and the amplification enzyme. Typically, amplification reagents along with other reaction components are mixed to form an amplification mixture which may be placed and contained in a reaction vessel (test tube, microwell, etc.).

As used herein, the terms “restriction endonucleases” and “restriction enzymes” refer to bacterial enzymes, each of which cut double-stranded DNA at or near a specific nucleotide sequence.

The terms “in operable combination,” “in operable order,” and “operably linked” as used herein refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced.

The term “isolated” when used in relation to a nucleic acid, as in “an isolated oligonucleotide” or “isolated polynucleotide” refers to a nucleic acid sequence that is identified and separated from at least one component or contaminant with which it is ordinarily associated in its natural source. Isolated nucleic acid is such present in a form or setting that is different from that in which it is found in nature. In contrast, non-isolated nucleic acids as nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding a given protein includes, by way of example, such nucleic acid in cells ordinarily expressing the given protein where the nucleic acid is in a chromosomal location different from that of natural cells, or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may be single-stranded), but may contain both the sense and anti-sense strands (i.e., the oligonucleotide or polynucleotide may be double-stranded).

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reaction that occur within a natural environment.

The term “test compound” refers to any chemical entity, pharmaceutical, drug, and the like that is a candidate for use to treat or prevent a disease, illness, sickness, or disorder of bodily function. Test compounds comprise both known and potential therapeutic compounds. A test compound can be determined to be therapeutic by screening using the screening methods of the present invention.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Environmental samples include environmental material such as surface matter, soil, water, crystals and industrial samples. Such examples are not however to be construed as limiting the sample types applicable to the present invention.

Advances in molecular biology are making an impact on the design and development of new, more efficient drugs, and more precise diagnostic procedures. However, there is still a noticeable gap when a given approach is already well established and widely used for research goals, but its clinical applications remain unrecognized and its usefulness for diagnostic and prognostic purposes remains untested.

Microarray-based expression profiling has emerged as a very powerful approach for broad evaluation of gene expression in various systems. However, this approach has its limitations, and one of the most important is the requirement of a certain minimal amount of mRNA: if it is below a certain level due to low promoter activity, short half-life of mRNA, or small amounts of starting material expression of the gene cannot be unambiguously detected. An additional concern is the stability of RNA, which in many cases is difficult to control (e.g., for surgically removed tissue samples), so that the absence of a signal for a certain gene might reflect artificially introduced degradation rather than genuine decrease in expression.

DNA is a much more stable milieu for analysis, and DNA methylation in regions with increased density of CpG dinucleotides (i.e., CpG islands) has been shown to correlate inversely with corresponding gene expression when such CpG islands are located in the promoter and/or the first exon of the gene. A number of techniques have been developed for methylation analysis; arguably the most popular of them—methylation-specific PCR or MSP—takes advantage of modification of unmethylated cytosines by bisulfate and alkali which results in their conversion to uracils, changing their partners from guanosine to thymidine. This change can be detected by PCR with primers that contain appropriate substitutions. A substantial amount of data on gene-specific methylation has been acquired using MSP.

The present invention improves methylation analysis by providing a technique for high throughput analysis without losses in the sensitivity. The first phase of the assay involves digestion of genomic DNA with methylation-sensitive enzyme (e.g., HpaII or Hin6I), which cuts unmethylated, for example, CCGG sites while leaving even hemi-methylated sites intact. Efficiency of this step determines the discriminating power of the approach, since the next procedure—amplification of the CpG island-containing fragment with primers flanking the methylation specific restriction enzyme site—serves mainly to increase the sensitivity of the assay. Reference is made to U.S. application Ser. No. 10/677,701, entitled “Methylation Profile of Cancer,” which was filed on Oct. 2, 2003, and claims the benefit of U.S. provisional application No. 60/415,628, filed on Oct. 2, 2002, the contents of which are incorporated herein by reference in their entireties.

The present invention overcomes many of the problems of mRNA arrays (e.g., stability of RNA and quantitation of expression) by evaluating gene expression by measuring methylation profiles of CpG islands. These regions of unusually high GC content have been described in many genes (Cooper et al., DNA 2:131 (1983)); the cytosine of CpG islands can be modified by methyltransferase to produce a methylated derivative-5-methylcytosine (Cooper et al., supra; Baylin et al., AIDS Res Hum Retroviruses 8:811 (1992)). If a methylated cytosine is located in the promoter region of a gene, it is likely to be silenced (Cooper et al., supra). Silencing of various tumor suppressor and growth regulator genes (Rountree et al., Oncogene. 20: 3156 (2001); Yang et at, Endocr Relat Cancer. 8: 115-127 (2001)) has been linked to cancer development and progression in general (Baylin et al., supra; Jones, Cancer Res. 46:461 (1986)). Accordingly, in some embodiments, present invention provides cancer diagnostics comprising the identification of methylation patterns in cancer samples. None of the known genes is methylated in all cases of cancer; thus simultaneous analysis of several genes within the same sample increases the clinical value of the assay.

In some embodiments, the present invention provides methylation-based procedures for cancer detection. The present invention demonstrates that microarray-mediated methylation assay (M³A) can achieve high sensitivity and high specificity. Importantly, M³A performance does not require subjective evaluation of assay data, making its results observer-independent.

Abnormal DNA methylation in neoplastic cells can be a valuable biomarker for cancer detection (Herman, 2004, Chest, 125:119 S-122S; Brena et al., 2006, J. Mol. Med. 1-13). Unfortunately, DNA of known regions has only a certain probability of methylation (Herman et al., 1995, Cancer Res. 55:4525-4530), and this probability varies for different stages of the disease (Kominsky et al., 2003, Oncogene 22:2021-2033; Fackler et al., 2003, Int. J. Cancer 107:970-975; Bae et al., 2004, Clin. Cancer Res. 10:5998-6005). To circumvent this problem, an approach based on evaluation of methylation in many regions within the same sample was developed, and statistical assessment of data from many clinical samples analyzed.

M³A was used for methylation detection. A limited number of GCGC sites in each gene is evaluated by this approach (Melnikov et al., 2005, Nucl. Acids Res. 33:e93), so, in some embodiments, choosing a different set of sites within the same set of genes can affect the final readout. Accordingly, in some embodiments, a variety of sets of sites within the same set of genes is utilized. This feature of the assay indicates that, in some embodiments, assignment of “methylated” or “unmethylated” values depends on the selection of the GCGC sites within each region.

Signal detection in M³A is based in part on competitive hybridization of two PCR products (one from digested and the second from undigested DNA of the same sample), which are labeled with different fluorophores, so that hybridization results are scored as fluorescence intensity for each of them. Assignment of “methylated” (M) and “unmethylated” (UM) calls depends on the ratio of fluorescence of undigested and digested DNA, which, in preferred embodiments, produce one of two values: 1, if the fragment is methylated and digestion does not affect its representation, and infinity, if the fragment is unmethylated and no signal from digested DNA is detected. This type of ideal distribution is rarely seen even in cell lines because of intrinsic heterogeneity of biological material (Melnikov et al., 2005, supra).

Additional complications may be associated with the unequal performance of fluorophores Cy3 and Cy5, which ideally should not influence signal distribution but in reality can affect the results. To adjust results a “self-self” hybridization is sometimes used for expression microarrays when aliquots of the same DNA sample are labeled separately with Cy3 and Cy5 fluorescent dyes and co-hybridized to the same microarray. Thus, in some embodiments, a similar adjustment is done for methylation detection, so the Cy5/Cy3 ratio from two identical aliquots can be used as the threshold of methylated fragments. Using this approach it is possible to convert numerical data of microarray experiments to binary readout defining methylated and unmethylated calls. In some embodiments, the technique is used for diagnostic purposes (e.g., for use with heterogeneous clinical samples where quantitative differences in methylation can depend on variations in tumor/stroma ratio, presence of inflammation, tumor cell death and other reasons).

In some embodiments, the present invention provides methods of correlating methylation patterns with clinical outcomes (e.g., patients at high-risk for developing cancer, disease-free survival, resistance to chemotherapy, and development of metastatic disease). In other embodiments, the present invention provides methods of disease monitoring during treatment and rapid screening of the high-risk population.

Differential methylation of CpG sequences provides an alternative way to characterize expression—or more accurately, repression—profiles of cell lines and tissues. Repression of heavily methylated genes is thought to depend on interactions of methylated cytosines with MeCP2, which either interferes with transcriptional complex assembly or prevents its movement.

Experiments conducted during the course of development of the present invention provide a novel methylation assay designed to provide a fast estimate on the methylation status of chosen genes. The assay relies on restriction endonuclease specificity to discriminate between methylated and unmethylated sequences, and on PCR reaction to amplify surviving templates. The present invention is not limited to the use of methylation specific restriction enzymes and PCR. Any method that examines methylation state (e.g., by selective cleavage, modification, etc.) followed by detection, is contemplated by the present invention. The number and specifics of the genes analyzed can be altered based on the choice of primers.

The methods of the present invention are amenable to detection of differences in expression profiles when inadequate quantities of starting material are available. In some embodiments, the method includes extensive digestion of genomic DNA with a methylation-sensitive restriction enzyme (e.g., HpaII or Hin6I), followed by multiplexed amplification of gene-specific DNA fragments comprising CpG sequences (e.g., CpG islands).

The markers of the present invention, when used to characterize or diagnose cancer, may be detected by any appropriate methodology or technology, including any future developed technologies that identify differentially methylated DNA sequences.

The present invention provides isolated antibodies. In some embodiments, the antibodies are used to confirm or validate the data obtained from methylation analysis. These antibodies find use in the diagnostic and therapeutic methods described herein.

In some embodiments, the present invention provides cancer therapies. In some embodiments, the cancer therapies target genes with altered methylation patterns in cancer, and in particular, breast, ovarian, lung, pancreatic, colon or prostate cancers.

In some embodiments, the present invention provides pharmaceutical compositions that may comprise all or portions of cancer markers polynucleotide sequences, cancer markers polypeptides, inhibitors or antagonists of cancer markers bioactivity, including antibodies, alone or in combination with at least one other agent, such as a stabilizing compound, and may be administered in any sterile, biocompatible pharmaceutical carrier, including, but not limited to, saline, buffered saline, dextrose, and water. The pharmaceutical compositions find use as therapeutic agents and vaccines for the treatment of cancer.

The present invention is not limited to the therapeutic applications described above. Indeed, any therapeutic application that specifically targets tumor cells expressing the cancer markers of the present invention are contemplated, including but not limited to, antisense therapies. In yet other embodiments, drugs that alter DNA methylation (e.g., demethylation drugs) are used to treat cancers that are identified by the methods of the present invention as comprising DNA hypermethylation. Exemplary demethylation drugs include, but are not limited to, those disclosed in Villar-Garea and Esteller (Current Drug Metabolism, 4:11 (2003)), Lin et al. (Cancer Research 61:8611 (2001)) and Young and Smith (J. Biol. Chem. 276:19610 (2001)).

The present invention provides methods and compositions for using cancer markers as a target for screening drugs that can alter, for example, expression of a cancer marker (e.g., those identified using the above methods) or methylation status of the cancer marker.

For example, in some embodiments, the methods of the present invention are used to evaluate the effect of drugs that alter DNA methylation status. In some embodiments, the methods of the present invention find use in the screening of candidate methylation drugs for efficacy and dosage. In other embodiments, the methods of the present invention are used to determine the specificity of drugs that effect DNA methylation (e.g., to determine the genes effected by DNA de-methylation drugs).

In particular, the present invention contemplates the use of cell lines transfected with cancer marker and variants thereof for screening compounds for activity, and in particular to high throughput screening of compounds from combinatorial libraries (e.g., libraries containing greater than 10⁴ compounds). The cell lines of the present invention can be used in a variety of screening methods. In some embodiments, the cells can be used in second messenger assays that monitor signal transduction following activation of cell-surface receptors. In other embodiments, the cells can be used in reporter gene assays that monitor cellular responses at the transcription/translation level. In still further embodiments, the cells can be used in cell proliferation assays to monitor the overall growth/no growth response of cells to external stimuli.

In second messenger assays, the host cells are preferably transfected as described above with vectors encoding cancer marker or variants or mutants thereof. The host cells are then treated with a compound or plurality of compounds (e.g., from a combinatorial library) and assayed for the presence or absence of a response. It is contemplated that at least some of the compounds in the combinatorial library can serve as agonists, antagonists, activators, or inhibitors of the expression or repression of cancer marker gene expression. It is also contemplated that at least some of the compounds in the combinatorial library can serve as agonists, antagonists, activators, or inhibitors of protein acting upstream or downstream of the protein encoded by the vector in a signal transduction pathway.

In some embodiments, the second messenger assays measure fluorescent signals from reporter molecules that respond to intracellular changes (e.g., Ca²⁺ concentration, membrane potential, pH, IP₃, cAMP, arachidonic acid release) due to stimulation of membrane receptors and ion channels (e.g., ligand gated ion channels; see Denyer et al., Drug Discov. Today 3:323 (1998); and Gonzales et al., Drug. Discov. Today 4:431-39 (1999)). Examples of reporter molecules include, but are not limited to, FRET (florescence resonance energy transfer) systems (e.g., Cuo-lipids and oxonols, EDAN/DABCYL), calcium sensitive indicators (e.g., Fluo-3, FURA 2, INDO 1, and FLUO3/AM, BAPTA AM), chloride-sensitive indicators (e.g., SPQ, SPA), potassium-sensitive indicators (e.g., PBFI), sodium-sensitive indicators (e.g., SBFI), and pH sensitive indicators (e.g., BCECF).

In general, the host cells are loaded with the indicator prior to exposure to the compound. Responses of the host cells to treatment with the compounds can be detected by methods known in the art, including, but not limited to, fluorescence microscopy, confocal microscopy (e.g., FCS systems), flow cytometry, microfluidic devices, FLIPR systems (See, e.g., Schroeder and Neagle, J. Biomol. Screening 1:75 (1996)), and plate-reading systems. In some preferred embodiments, the response (e.g., increase in fluorescent intensity) caused by compound of unknown activity is compared to the response generated by a known agonist and expressed as a percentage of the maximal response of the known agonist. The maximum response caused by a known agonist is defined as a 100% response. Likewise, the maximal response recorded after addition of an agonist to a sample containing a known or test antagonist is detectably lower than the 100% response.

The cells are also useful in reporter gene assays. Reporter gene assays involve the use of host cells transfected with vectors encoding a nucleic acid comprising transcriptional control elements of a target gene (i.e., a gene that controls the biological expression and function of a disease target) spliced to a coding sequence for a reporter gene. Therefore, activation of the target gene results in activation of the reporter gene product. In some embodiments, the reporter gene construct comprises the 5′ regulatory region (e.g., promoters and/or enhancers) of a protein whose expression is controlled by cancer marker in operable association with a reporter gene. Examples of reporter genes finding use in the present invention include, but are not limited to, chloramphenicol transferase, alkaline phosphatase, firefly and bacterial luciferases, β-galactosidase, β-lactamase, and green fluorescent protein. The production of these proteins, with the exception of green fluorescent protein, is detected through the use of chemiluminescent, colorimetric, or bioluminecent products of specific substrates (e.g., X-gal and luciferin). Comparisons between compounds of known and unknown activities may be conducted as described above.

Specifically, the present invention provides screening methods for identifying modulators, i.e., candidate or test compounds or agents (e.g., proteins, peptides, peptidomimetics, peptoids, small molecules or other drugs) which bind to cancer markers of the present invention or regulate the expression of cancer markers of the present invention, have an inhibitory (or stimulatory) effect on, for example, cancer marker expression or cancer marker activity, or have a stimulatory or inhibitory effect on, for example, the expression or activity of a cancer marker substrate. Compounds thus identified can be used to modulate the activity of target gene products (e.g., cancer marker genes) either directly or indirectly in a therapeutic protocol, to elaborate the biological function of the target gene product, or to identify compounds that disrupt normal target gene interactions. Compounds that alter the expression of a cancer marker of the present invention are particularly useful in the treatment of cancers.

In one embodiment, the invention provides assays for screening candidate or test compounds that are substrates of a cancer marker protein or polypeptide or a biologically active portion thereof. In another embodiment, the invention provides assays for screening candidate or test compounds that bind to or modulate the activity of a cancer marker protein or polypeptide or a biologically active portion thereof.

The test compounds of the present invention can be obtained using any of the numerous approaches in combinatorial library methods known in the art, including biological libraries; peptoid libraries (libraries of molecules having the functionalities of peptides, but with a novel, non-peptide backbone, which are resistant to enzymatic degradation but which nevertheless remain bioactive; see, e.g., Zuckennann et al., J. Med. Chem. 37: 2678-85 (1994)); spatially addressable parallel solid phase or solution phase libraries; synthetic library methods requiring deconvolution; the ‘one-bead one-compound’ library method; and synthetic library methods using affinity chromatography selection. The biological library and peptoid library approaches are preferred for use with peptide libraries, while the other four approaches are applicable to peptide, non-peptide oligomer or small molecule libraries of compounds (Lam (1997) Anticancer Drug Des. 12:145).

Examples of methods for the synthesis of molecular libraries can be found in the art, for example in: DeWitt et al., Proc. Natl. Acad. Sci. U.S.A. 90:6909 (1993); Erb et al., Proc. Nad. Acad. Sci. USA 91:11422 (1994); Zuckermann et al., J. Med. Chem. 37:2678 (1994); Cho et al., Science 261:1303 (1993); Carrell et al., Angew. Chem. Int. Ed. Engl. 33.2059 (1994); Carell et al., Angew. Chem. Int. Ed. Engl. 33:2061 (1994); and Gallop et al., J. Med. Chem. 37:1233 (1994).

Libraries of compounds may be presented in solution (e.g., Houghten, Biotechniques 13:412-421 (1992)), or on beads (Lain, Nature 354:82-84 (1991)), chips (Fodor, Nature 364:555-556 (1993)), bacteria or spores (U.S. Pat. No. 5,223,409; herein incorporated by reference), plasmids (Cull et W., Proc. Nad. Acad. Sci. USA 89:18651869 (1992)) or on phage (Scott and Smith, Science 249:386-390 (1990); Devlin Science 249:404-406 (1990); Cwirla et al., Proc. Natl Acad. Sci. 87:6378-6382 (1990); Felici, J. Mol. Biol. 222:301 (1991)).

In one embodiment, an assay is a cell-based assay in which a cell that expresses a cancer marker protein or biologically active portion thereof is contacted with a test compound, and the ability of the test compound to the modulate cancer marker's activity or expression is determined. Determining the ability of the test compound to modulate cancer marker activity can be accomplished by monitoring, for example, changes in enzymatic activity. The cell, for example, can be of mammalian origin.

The ability of the test compound to modulate cancer marker binding to a compound, e.g., a cancer marker substrate, can also be evaluated. This can be accomplished, for example, by coupling the compound, e.g., the substrate, with a radioisotope or enzymatic label such that binding of the compound, e.g., the substrate, to a cancer marker can be determined by detecting the labeled compound, e.g., substrate, in a complex.

Alternatively, the cancer marker is coupled with a radioisotope or enzymatic label to monitor the ability of a test compound to modulate cancer marker binding to a cancer marker substrate in a complex. For example, compounds (e.g., substrates) can be labeled with ¹²⁵I, ³⁵S, ¹⁴C or ³H, either directly or indirectly, and the radioisotope detected by direct counting of radioemmission or by scintillation counting. Alternatively, compounds can be enzymatically labeled with, for example, horseradish peroxidase, alkaline phosphatase, or luciferase, and the enzymatic label, detected by determination of conversion of an appropriate substrate to product.

The ability of a compound (e.g., a cancer marker substrate) to interact with a cancer marker with or without the labeling of any of the interactants can be evaluated. For example, a microphysiometer can be used to detect the interaction of a compound with a cancer marker without the labeling of either the compound or the cancer marker (McConnell et al. Science 257:1906-1912 (1992)). As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and cancer marker.

In yet another embodiment, a cell-free assay is provided in which a cancer marker gene, protein or biologically active portion thereof is contacted with a test compound and the ability of the test compound to bind to the cancer marker gene, protein or biologically active portion thereof is evaluated. Preferred biologically active portions of the cancer marker proteins to be used in assays of the present invention include fragments that participate in interactions with substrates or other proteins, e.g., fragments with high surface probability scores.

Cell-free assays involve preparing a reaction mixture of the target gene protein and the test compound under conditions and for a time sufficient to allow the two components to interact and bind, thus forming a complex that can be removed and/or detected.

The interaction between two molecules can also be detected, e.g., using fluorescence energy transfer (FRET) (see, for example, Lakowicz et al., U.S. Pat. No. 5,631,169; Stavrianopoulos et al., U.S. Pat. No. 4,968,103; each of which is herein incorporated by reference). In another embodiment, determining the ability of the cancer marker protein or nucleic acid to bind to a target molecule can be accomplished using real-time Biomolecular Interaction Analysis (BIA) (see, e.g., Sjolander and Urbaniczky, Anal. Chem. 63:2338-2345 (1991) and Szabo et al. Curr. Opin. Struct. Biol. 5:699-705 (1995)). “Surface plasmon resonance” or “BIA” detects biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal that can be used as an indication of real-time reactions between biological molecules.

In one embodiment, the target gene product or the test substance is anchored onto a solid phase. The target gene product/test compound complexes anchored on the solid phase can be detected at the end of the reaction. Preferably, the target gene product can be anchored onto a solid surface, and the test compound, (which is not anchored), can be labeled, either directly or indirectly, with detectable labels discussed herein.

It may be desirable to immobilize cancer marker nucleic acids, proteins, an anti-cancer marker antibody or its target molecule to facilitate separation of complexed from non-complexed forms of one or both of the proteins, as well as to accommodate automation of the assay. Binding of a test compound to a cancer marker protein, or interaction of a cancer marker protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix. For example, glutathione-S-transferase-cancer marker fusion proteins or glutathione-S-transferase/target fusion proteins can be adsorbed onto glutathione Sepharose beads (Sigma Chemical, St. Louis, Mo.) or glutathione-derivatized microtiter plates, which are then combined with the test compound or the test compound and either the non-adsorbed target protein or cancer marker protein, and the mixture incubated under conditions conducive for complex formation (e.g., at physiological conditions for salt and pH). Following incubation, the beads or microtiter plate wells are washed to remove any unbound components, the matrix immobilized in the case of beads, complex determined either directly or indirectly, for example, as described above.

Alternatively, the complexes can be dissociated from the matrix, and the level of cancer marker binding or activity determined using standard techniques. Other techniques for immobilizing either cancer marker protein or a target molecule on matrices include using conjugation of biotin and streptavidin. Biotinylated cancer marker protein or target molecules can be prepared from biotin-NHS (N-hydroxy-succinimide) using techniques known in the art (e.g., biotinylation kit, Pierce Chemicals, Rockford, EL), and immobilized in the wells of streptavidin-coated 96 well plates (Pierce Chemical).

In order to conduct the assay, the non-immobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface; e.g., using a labeled antibody specific for the immobilized component (the antibody, in turn, can be directly labeled or indirectly labeled with, e.g., a labeled anti-IgG antibody).

This assay is performed utilizing antibodies reactive with cancer marker protein or target molecules but which do not interfere with binding of the cancer marker protein to its target molecule. Such antibodies can be derivatized to the wells of the plate, and unbound target or cancer marker protein trapped in the wells by antibody conjugation. Methods for detecting such complexes, in addition to those described above for the GST-immobilized complexes, include immunodetection of complexes using antibodies reactive with the cancer marker protein or target molecule, as well as enzyme-linked assays which rely on detecting an enzymatic activity associated with the cancer marker protein or target molecule.

Alternatively, cell free assays can be conducted in a liquid phase. In such an assay, the reaction products are separated from unreacted components, by any of a number of standard techniques, including, but not limited to: differential centrifugation (see, for example, Rivas and Minton, Trends Biochem Sci 18:284-7 (1993)); chromatography (gel filtration chromatography, ion-exchange chromatography); electrophoresis (see, e.g., Ausubel et al., eds. Current Protocols in Molecular Biology 1999, J. Wiley: New York.); and immunoprecipitation (see, for example, Ausubel et al., eds. Current Protocols in Molecular Biology 1999, J. Wiley: New York). Such resins and chromatographic techniques are known to one skilled in the art (See e.g., Heegaard J. Mol. Recognit. 11:141-8 (1998); Hageand Tweed J. Chromatogr. Biomed. Sci. Appl 699:499-525 (1997)). Further, fluorescence energy transfer may also be conveniently utilized, as described herein, to detect binding without further purification of the complex from solution.

The assay can include contacting the cancer marker nucleic acid, protein or biologically active portion thereof with a known compound that binds the cancer marker to form an assay mixture, contacting the assay mixture with a test compound, and determining the ability of the test compound to interact with a cancer marker protein, wherein determining the ability of the test compound to interact with a cancer marker protein includes determining the ability of the test compound to preferentially bind to cancer marker or biologically active portion thereof, or to modulate the activity of a target molecule, as compared to the known compound.

To the extent that cancer marker can, in vivo, interact with one or more cellular or extracellular macromolecules, such as proteins, inhibitors of such an interaction are useful. A homogeneous assay can be used to identify inhibitors.

Modulators of cancer marker expression can also be identified. For example, a cell or cell free mixture is contacted with a candidate compound and the expression of cancer marker mRNA or protein evaluated relative to the level of expression of cancer marker mRNA or protein in the absence of the candidate compound. When expression of cancer marker mRNA or protein is greater in the presence of the candidate compound than in its absence, the candidate compound is identified as a stimulator of cancer marker mRNA or protein expression. Alternatively, when expression of cancer marker mRNA or protein is less (i.e., statistically significantly less) in the presence of the candidate compound than in its absence, the candidate compound is identified as an inhibitor of cancer marker mRNA or protein expression. The level of cancer marker mRNA or protein expression can be determined by methods described herein for detecting cancer marker mRNA or protein.

A modulating agent can be identified using a cell-based or a cell free assay, and the ability of the agent to modulate the activity of a cancer marker protein can be confirmed in vivo, e.g., in an animal such as an animal model for a disease (e.g., an animal with breast cancer).

The present invention contemplates the generation of transgenic animals comprising an exogenous cancer marker gene of the present invention or mutants and variants thereof (e.g., truncations). In preferred embodiments, the transgenic animal displays an altered phenotype (e.g., increased presence of cancer or drug resistant cancer) as compared to wild-type animals. Methods for analyzing the presence or absence of such phenotypes include but are not limited to, those disclosed herein. In some preferred embodiments, the transgenic animals further display an increased growth of tumors or increased evidence of cancer.

The transgenic animals of the present invention find use in drug (e.g., cancer therapy) screens. In some embodiments, test compounds (e.g., a drug that is suspected of being useful to treat cancer) and control compounds (e.g., a placebo) are administered to the transgenic animals and the control animals and the effects evaluated. In other embodiments, transgenic and control animals are given immunotherapy (e.g., including but not limited to, the methods described above) and the effect on cancer symptoms is assessed.

The transgenic animals can be generated via a variety of methods. In some embodiments, embryonal cells at various developmental stages are used to introduce transgenes for the production of transgenic animals. Different methods are used depending on the stage of development of the embryonal cell. The zygote is the best target for micro-injection. In the mouse, the male pronucleus reaches the size of approximately 20 micrometers in diameter, which allows reproducible injection of 1-2 picoliters (pl) of DNA solution. The use of zygotes as a target for gene transfer has a major advantage in that in most cases the injected DNA will be incorporated into the host genome before the first cleavage (Brinster et al., Proc. Natl. Acad. Sci. USA 82:4438-4442 (1985)). As a consequence, all cells of the transgenic non-human animal will carry the incorporated transgene. This will in general also be reflected in the efficient transmission of the transgene to offspring of the founder since 50% of the germ cells will harbor the transgene. U.S. Pat. No. 4,873,191 describes a method for the micro-injection of zygotes; the disclosure of this patent is incorporated herein in its entirety.

In other embodiments, retroviral infection is used to introduce transgenes into a non-human animal. In still other embodiments, homologous recombination is utilized to knock-out gene function or create deletion mutants (e.g., truncation mutants). Methods for homologous recombination are described in U.S. Pat. No. 5,614,396, incorporated herein by reference.

### Illustrative Embodiments

The following embodiments are provided in order to demonstrate and further illustrate certain preferred aspects of the present invention and are not to be construed as limiting the scope thereof.

Embodiment 1. A method for detecting cancer in a subject, comprising: a) providing a sample from said subject, wherein said sample comprises nucleic acid; b) exposing said sample to reagents for detecting methylation status; and c) determining the methylation status of the promoter of a gene listed in Table 1.

Embodiment 2. A method of characterizing cancer, comprising: a) providing a sample from a subject, said sample comprising genomic DNA; and b) detecting the presence or absence of DNA methylation in five or more genes listed in Table 1, thereby characterizing cancer in said subject.

Embodiment 3. The method of embodiment 1, wherein said detecting cancer comprises detecting the presence or absence of breast cancer.

Embodiment 4. The method of embodiment 1, wherein said detecting cancer comprises detecting the presence or absence of ovarian cancer.

Embodiment 5. The method of embodiment 1, wherein said detecting cancer comprises detecting the presence or absence of lung cancer.

Embodiment 6. The method of embodiment 1, wherein said detecting cancer comprises detecting the presence or absence of pancreatic cancer.

Embodiment 7. The method of embodiment 1, wherein said detecting cancer comprises detecting the presence or absence of colon cancer.

Embodiment 8. The method of embodiment 1, wherein said detecting cancer comprises detecting the presence or absence of prostate cancer.

Embodiment 9. The method of embodiment 1, wherein said sample is plasma.

Embodiment 10. The method of embodiment 2, wherein said sample is plasma.

Embodiment 11. The method of embodiment 1 or 2, wherein said DNA methylation comprises CpG methylation.

Embodiment 12. The method of embodiment 2, wherein said cancer is breast cancer.

Embodiment 13. The method of embodiment 2, wherein said cancer is ovarian cancer.

Embodiment 14. The method of embodiment 2, wherein said cancer is long cancer.

Embodiment 15. The method of embodiment 2, wherein said cancer is pancreatic cancer.

Embodiment 16. The method of embodiment 2, wherein said cancer is colon cancer.

Embodiment 17. The method of embodiment 2, wherein said cancer is prostate cancer.

Embodiment 18. A kit for characterizing cancer, comprising reagents sufficient for detecting the presence or absence of DNA methylation from a blood sample in five or more genes listed in Table 1.

Embodiment 19. The kit of embodiment 18, further comprising reagents for detecting the presence or absence of DNA methylation of eight or more genes listed in Table 1.

Embodiment 20. The kit of embodiment 18, further comprising instructions for using said kit for characterizing cancer in said subject.

Embodiment 21. A method for diagnosing cancer in a subject, comprising: (a) reacting isolated genomic DNA from the subject and a methylation-sensitive restriction enzyme; wherein the genomic DNA comprises a plurality of promoters from different genes, and the enzyme cleaves unmethylated CpG sequences in the promoters and does not cleave methylated CpG sequences in the promoters; (b) contacting the genomic DNA thus reacted and a plurality of pairs of specific primers in a multiplex amplification mixture, the pairs of specific primers being configured to hybridize to the genomic DNA and to amplify a plurality of different promoters through a region comprising an uncleaved CpG sequence; (c) reacting the amplification mixture; (d) detecting one or more amplified promoters in the reacted amplification mixture or the absence thereof, thereby diagnosing cancer in the subject selected from the group consisting of ovarian cancer, lung cancer, prostate cancer, pancreatic cancer, and colon cancer.

Embodiment 22. The method of embodiment 21, wherein the genomic DNA is isolated from blood.

Embodiment 23. The method of embodiment 21, wherein the genomic DNA is isolated from plasma.

Embodiment 24. The method of embodiment 21, wherein the genomic DNA is isolated from tissue of the subject.

Embodiment 25. The method of any of embodiments 21-24, wherein detecting one or more amplified promoters in the reacted amplification mixture or the absence thereof comprises: (1) contacting a microarray and the reacted amplification mixture, the microarray comprising a plurality of DNA samples, each of which hybridizes to one of the plurality of different promoters; and (2) detecting hybridization or the lack of hybridization between DNA in the reacted amplification mixture and one or more of the plurality of DNA samples of the microarray thereby obtaining a methylation profile.

Embodiment 26. The method of embodiment 25, further comprising comparing the methylation profile for the subject and a standard methylation profile selected from the group consisting of a standard methylation profile for non-cancerous samples, a standard methylation profile for cancerous samples, and both standard methylation profiles.

Embodiment 27. The method of embodiment any of embodiments 21-26, further comprising the step of separating the isolated genomic DNA of step (a) into: (i) a control sample and (ii) an experimental sample and adding control nucleic acid to both the control and experimental samples, wherein the control nucleic acid comprises at least one known CpG sequence that is unmethylated.

Embodiment 28. The method of embodiment 27, wherein the control sample is not reacted with the methylation-sensitive restriction enzyme and the experimental sample is reacted with the methylation-sensitive restriction enzyme, and wherein both the control and experimental samples are contacted with primers for the control nucleic acid under conditions such that a fragment of the control nucleic acid is amplified if the known CpG sequence is uncleaved.

Embodiment 29. The method of any of embodiments 21-28, wherein the plurality of pairs of specific primers comprises at least five pairs of specific primers.

Embodiment 30. The method of embodiment 29, wherein each of the five pairs of specific primers is configured to amplify a gene selected from the group consisting of FHIT, HMLH1, DNAJC15, MGMT, progesterone receptor (e.g., PR-1P or PR-2D), RARB, RPL15, PYCARD, and PLAU, and the diagnosed cancer is ovarian cancer.

Embodiment 31. The method of embodiment 29, wherein each of the five pairs of specific primers is configured to amplify a gene selected from the group consisting of BRCA1, EP300, NR3C1 (GR), MLH1, DNAJC15 (MCJ), CDKN1C (p57kip2), TP73, PGR (proximal promoter), THBS1, and PYCARD (TMS1), and the diagnosed cancer is ovarian cancer.

Embodiment 32. The method of embodiment 29, wherein each of the five pairs of specific primers is configured to amplify a gene selected from the group consisting of BRCA1, HIC1, PAX5, PGR (proximal promoter), and THBS1, and the diagnosed cancer is ovarian cancer.

Embodiment 33. The method of embodiment 29, wherein the five pairs of specific primers comprise a primer pair that is configured to amplify a promoter of a gene selected from the group consisting of FHIT, MLH1, DNAJC15, MGMT, progesterone receptor (e.g., PR-1P or PR-2D), RARB, RPL15, PYCARD, and PLAU, and the diagnosed cancer is ovarian cancer.

Embodiment 34. The method of embodiment 29, wherein each of the five pairs of specific primers is configured to amplify a gene selected from the group consisting of CASP 8, CDKN1C, VHL, PAX5, DAPK1, NR3C1, MGMT, progesterone receptor (e.g., PR-1P or PR-2D), MLH1, RFC, TES, TNFSF11, CCND2, MYOD1, RB1, SFN, ESR1 (e.g., promoter A or promoter B), and GPC3, and the diagnosed cancer is lung cancer.

Embodiment 35. The method of embodiment 29, wherein each of the five pairs of specific primers is configured to amplify a gene selected from the group consisting of CASP 8, CDKN1C, VHL, PAX5, progesterone receptor (e.g., PR-1P or PR-2D), and GPC3, and the diagnosed cancer is lung cancer.

Embodiment 36. The method of embodiment 29, wherein the five pairs of specific primers comprise a primer pair that is configured to amplify a promoter of a gene selected from the group consisting of CASP 8, CDKN1C, VHL, PAX5, DAPK1, NR3C1, MGMT, progesterone receptor (e.g., PR-1P or PR-2D), MLH1, RFC, TES, TNFSF11, CCND2, MYOD1, RB1, SFN, ESR1 (e.g., promoter A or promoter B), and GPC3, and the diagnosed cancer is lung cancer.

Embodiment 37. The method of embodiment 29, wherein the five pairs of specific primers comprise a primer pair that is configured to amplify a promoter of a gene selected from the group consisting of CASP 8, CDKN1C, VHL, PAX5, progesterone receptor (e.g., PR-1P or PR-2D), and GPC3, and the diagnosed cancer is lung cancer.

Embodiment 38. The method of embodiment 29, wherein each of the five pairs of specific primers is configured to amplify a gene selected from the group consisting of BRCA1, CALCA, CASP 8, CCND2, EDNRB, EP 300, FHIT, GPC3, NR3C1, HIC1, DNAJC15, FABP3, ABCB1, MSH2, CDKN1A, CDKN1C, PAX5, PGK1, progesterone receptor (e.g., PR-1P or PR-2D), S100A2, TES, THBS, and VHL, and the diagnosed cancer is prostate cancer.

Embodiment 39. The method of embodiment 29, wherein the five pairs of specific primers comprise a primer pair that is configured to amplify a promoter of a gene selected from the group consisting of BRCA1, CALCA, CASP 8, CCND2, EDNRB, EP 300, FHIT, GPC3, NR3C1, HIC1, DNAJC15, FABP3, ABCB1, MSH2, CDKN1A, CDKN1C, PAX5, PGK1, progesterone receptor (e.g., PR-1P or PR-2D), S100A2, TES, THBS, and VHL, and the diagnosed cancer is prostate cancer.

Embodiment 40. The method of embodiment 29, wherein each of the five pairs of specific primers is configured to amplify a gene selected from the group consisting of SFN, BRCA1, DAPK1, EDNRB, NR3C1, DNAJC15, MUC2, CDKN1A, CDKN1C, PGK1, progesterone receptor (e.g., PR-1P or PR-2D), S100A2, TES, and VHL, and the diagnosed cancer is pancreatic cancer.

Embodiment 41. The method of embodiment 29, wherein the five pairs of specific primers comprise a primer pair that is configured to amplify a promoter of a gene selected from the group consisting of SFN, BRCA1, DAPK1, EDNRB, NR3C1, DNAJC15, MUC2, CDKN1A, CDKN1C, PGK1, progesterone receptor (e.g., PR-1P or PR-2D), S100A2, TES, and VHL, and the diagnosed cancer is pancreatic cancer.

Embodiment 42. The method of embodiment 29, wherein each of the five pairs of specific primers is configured to amplify a gene selected from the group consisting of BRCA1, CASP 8, CCND2, DAPK1, ESR1 (e.g., promoter A or promoter B), GPC3, NR3C1, ABCB1, MYOD1, CDKN1A, CDKN1C, PGK1, progesterone receptor (e.g., PR-1P or PR-2D), RAR, RB1, RFC, RPL15, S100A2, SOCS1, TES, THBS, and VHL, and the diagnosed cancer is colon cancer.

Embodiment 43. The method of embodiment 29, wherein the five pairs of specific primers comprise a primer pair that is configured to amplify a promoter of a gene selected from the group consisting of BRCA1, CASP 8, CCND2, DAPK1, ESR1 (e.g., promoter A or promoter B), GPC3, NR3C1, ABCB1, MYOD1, CDKN1A, CDKN1C, PGK1, progesterone receptor (e.g., PR-1P or PR-2D), RAR, RB1, RcC, RPL15, S100A2, SOCS1, TES, THBS, and VHL, and the diagnosed cancer is colon cancer.

Embodiment 44. The method of any of embodiments 21-43, wherein the plurality of pairs of specific primers comprises at least ten pairs of specific primers.

Embodiment 45. The method of any of embodiments 21-43, wherein the plurality of pairs of specific primers comprises at least forty pairs of specific primers.

Embodiment 46. The method of any of embodiments 21-45, wherein the methylation specific restriction enzyme comprises Hin6I.

Embodiment 47. The method of any of embodiments 21-46, (a) reacting isolated genomic DNA from the subject and the methylation-sensitive restriction enzyme comprises digesting the genomic DNA to completion.

Embodiment 48. The method of any of embodiments 21-46, wherein diagnosing cancer comprises diagnosing the presence of chemotherapy resistant cancer.

Embodiment 49. The method of any of embodiments 21-46, wherein diagnosing cancer comprises determining chance of disease-free survival.

Embodiment 50. The method of any of embodiments 21-46, wherein diagnosing cancer comprises determining risk of developing metastatic disease.

Embodiment 51. The method of any of embodiments 21-46, wherein diagnosing cancer comprises monitoring disease progression in the subject.

Embodiment 52. The method of any of embodiments 21-51, wherein the method diagnoses cancer with a sensitivity of at least about 80%, preferably at least about 90%, more preferably at least about 95%.

Embodiment 53. A method for diagnosing pancreatic cancer in a subject, comprising: (a) reacting a plasma sample from the subject and reagents for detecting methylation status of genomic DNA in the sample; (b) determining the methylation status for a plurality of genes to generate a methylation profile, thereby diagnosing pancreatic cancer in the subject.

Embodiment 54. A method for diagnosing colon cancer in a subject, comprising: (a) reacting a plasma sample from the subject and reagents for detecting methylation status of genomic DNA in the sample; (b) determining the methylation status for a plurality of genes to generate a methylation profile, thereby diagnosing colon cancer in the subject.

Embodiment 55. The method of embodiment 53 or 54, wherein the method diagnoses cancer with a sensitivity of at least about 80%, preferably at least about 90%, more preferably at least about 95%.

Embodiment 56. A method for diagnosing hyperplasia in breast tissue of a subject, comprising: (a) reacting isolated genomic DNA from the subject and a methylation-sensitive restriction enzyme; wherein the genomic DNA comprises a plurality of promoters from different genes, and the enzyme cleaves an unmethylated CpG sequence in the promoters and does not cleave a methylated CpG sequence in the promoters; (c) contacting the genomic DNA thus reacted and a plurality of pairs of specific primers in a multiplex amplification mixture, the pairs of specific primers being configured to hybridize to the genomic DNA and to amplify a plurality of different promoters through a region comprising an uncleaved CpG sequence; (d) reacting the amplification mixture; (e) detecting one or more amplified promoters in the reacted amplification mixture or the absence thereof, thereby diagnosing hyperplasia in breast tissue of the subject, wherein the diagnosed hyperplasia in breast tissue is selected from the group consisting of invasive ductal carcinoma (IDC), ductal carcinoma in situ (DCIS), atypical ductal hyperplasia (ADH), and combinations thereof.

Embodiment 57. The method of embodiment 56, wherein the genomic DNA is isolated from breast tissue of the subject.

Embodiment 58. The method of embodiment 56, wherein the genomic DNA is isolated from ductal fluid of the subject.

Embodiment 59. The method of any of embodiments 56-58, wherein detecting one or more amplified promoters in the reacted amplification mixture or the absence thereof comprises: (1) contacting a microarray and the reacted amplification mixture, the microarray comprising a plurality of DNA samples, each of which hybridizes to one of the plurality of different promoters; and (2) detecting hybridization or the lack of hybridization between DNA in the reacted amplification mixture and one or more of the plurality of DNA samples of the microarray thereby obtaining a methylation profile.

Embodiment 60. The method of embodiment 59, further comprising comparing the methylation profile for the subject and a standard methylation profile selected from the group consisting of a standard methylation profile for non-cancerous samples, a standard methylation profile for cancerous samples, and both standard methylation profiles.

Embodiment 61. The method of any of embodiments 56-60, further comprising the step of separating the isolated genomic DNA of step (a) into: (i) a control sample and (ii) an experimental sample and adding control nucleic acid to both the control and experimental samples, wherein the control nucleic acid comprises at least one known CpG sequence that is umethylated.

Embodiment 62. The method of embodiment 61, wherein the control sample is not reacted with the methylation-sensitive restriction enzyme and the experimental sample is reacted with the methylation-sensitive restriction enzyme, and wherein both the control and experimental samples are contacted with primers for the control nucleic acid under conditions such that a fragment of the control nucleic acid is amplified if the known CpG sequence is uncleaved.

Embodiment 63. The method of any of embodiments 56-62, wherein the plurality of pairs of specific primers comprises at least five pairs of specific primers.

Embodiment 64. The method of embodiment 63, wherein each of the five pairs of specific primers is configured to amplify a gene selected from the group consisting of EP300, MGMT, TP73, PGR (distal promoter), THBS1, PYCARD (TMS1), PRKCDBP (SRBC), FABP3 (MDG1), MSH2, HIC1, BRCA1, TES, NR3C1 (GR), ICAM1, DAPK1, TNFSF11 (RANKL), DNAJC15 (MCJ), CDH1, CASP8, RPL15, and PGK1.

Embodiment 65. The method of embodiment 64, wherein the five pairs of specific primers comprise a primer pair that is configured to amplify a promoter of a gene selected from the group consisting of EP300, MGMT, TP73, PGR (distal promoter), THBS1, PYCARD (TMS1), PRKCDBP (SRBC), FABP3 (MDG1), MSH2, HIC1, BRCA1, TES, NR3C1 (GR), ICAM1, DAPK1, TNFSF11 (RANKL), DNAJC15 (MCJ), CDH1, CASP8, RPL15, and PGK1.

Embodiment 66. The method of any of embodiments 56-65, wherein the plurality of pairs of specific primers comprises at least ten pairs of specific primers.

Embodiment 67. The method of any of embodiments 56-66, wherein the method diagnoses cancer with a sensitivity of at least about 80%, preferably at least about 90%, more preferably at least about 95%.

Embodiment 68. A kit for performing any of the methods of embodiments 21-66.

### EXAMPLES

The following Examples (I-III) are provided in order to demonstrate and further illustrate certain preferred embodiments and aspects of the present invention and are not to be construed as limiting the scope thereof.

### Example I

### A. Experimental

1. General Experimental Outline

Purified genomic DNA from tumor specific plasma samples is divided into two parts; one of the samples is treated with the methylation-sensitive restriction enzyme Hin6I while the other one is used as a control. Both control and digested DNA is used as templates for nested PCR with aminoallyl-dUTP added at the second round of amplification. Following amplification, the incorporated aminoallyl-dUTP is coupled to reactive Cy5 or Cy3 dyes, creating fluorescently labeled probes. One of the dyes is used for PCR products from undigested control DNA, while another is used for PCR products from Hin6I-digested DNA. Both labeled products are mixed together and applied to a custom-designed microarray slide for competitive hybridization. A microarray reader is used to quantify fluorescence of each fluorophore in every spot of the array, and the Cy5/Cy3 ratio used to assess methylation status. Methylated fragments produce Cy5/Cy3 ratios close to 1, while unmethylated fragments have ratios higher than 1. Statistical analysis of hybridization data is performed to identify informative features and build the classifier for each cancer marker panel.

2. DNA Isolation from Plasma

Plasma (100 μl) was incubated with 1 ml DNAzol (MRC, Inc.) for 15 min at room temperature. NaCl (0.15 M final concentration), EDTA (1.5 mM final concentration) and linear polyacrylamide (80 μg/ml final concentration) were added to the plasma/DNAzol mix and the solution was thoroughly mixed followed by DNA precipitation with 0.5 ml ethanol. The DNA was pelleted by microcentrifuge at 12000 rpm for 10 min at room temperature. The DNA pellet was dissolved pellet in 50 μl buffer (10 mM Tris pH 8.0, 5 mM EDTA, 50 mM NaCl and 150 μg/ml proteinase K), and the DNA sample was incubated at 55° C. for 2 hr. DNAzol treatment and DNA precipitation was repeated as above, and the final pellet was washed twice with 70% ethanol. The final, washed DNA pellet was dissolved in 40 μl of 8 mM NaOH, and the solution was neutralized with 1M Hepes. DNA concentration was measured with DNA Quant 200 (Hoefer) instrument.

3. Restriction Enzyme Digestion of Tissues

Exhaustive digestion of DNA is done with the methylation sensitive restriction endonuclease Hin6I (Fermentas International, Inc., recognition site GCGC). Successful digestion of 4 ng of DNA is done with 40 U of the enzyme in 100 μl of reaction mix at 37° C. for 48 hr. To exclude non-specific degradation of DNA during a long incubation we use the second aliquot of DNA incubated without the enzyme. This control is then processed side-by-side with digested DNA and only fragments with an adequate signal from control DNA are scored. After digestion is completed, the DNA is purified and quantitated as previously described.

4. PCR Amplification of Sample DNA

The first round of PCR amplification (see Table 2 for primer sequences; F=forward primer, R=reverse primer) is performed using 400 pg of digested and control DNAs. Empirically assembled primer groups for multiplex reactions allow simultaneous amplification of five targets in each reaction. Final concentration of primers is 0.2 μM for each of the multiplex PCR reactions. KlenTaq®1 (DNA Polymerase Technology, Inc) is used at 20 Upper 50 μl reaction. To PCR buffer supplied with the enzyme we add betaine (Sigma) to 1.5M and dNTPs (Sigma) to 0.25 mM. The tubes are placed into a preheated ABI 9600 thermocycler and incubated for 5 min prior to addition of KlenTaq® 1. PCR is started for 25 cycles by initial denaturation at 95° C. followed by 25 cycles of; 45 sec—62° C.; 1 min—72° C.; 1 min cycling conditions. After 25 cycles the PCR reactions are kept at 4° C.

The PCR products of the first round are purified using QIAquick® PCR Purification Kit (Qiagen) and quantified. Amplification products for corresponding DNAs are combined, and 400 pg are used for the second PCR, which is assembled as above except for dNTPs, where a mix of aminoallyl-dUTP (Biotium, Inc,) and dTTP (3:1) is used. The second round of PCR (see Table 3 for primer sequences; F=forward primer, R=reverse primer) is performed as the first except only 20 cycles are used. PCR products are purified using QIAquick PCR Purification Kit and products are combined.

The second PCR products are dried in vacuum and dissolved in 5 μl of 200 mM NaHCO₃ buffer (pH 9.0). Cy3 or Cy5 fluorescent dyes in DMSO are added to each tube, mixed and spun. Labeling continues for two hours at room temperature in the dark. Unreacted Cy dyes are quenched by 4.5 μl 4M hydroxylamine for 15 minutes in the dark. Final purification is done by precipitating labeled PCR products with ethanol.

TABLE 2

| Gene | Primer 5′ to 3′ | SEQ ID NO |
|---|---|---|
| SFN-F | TGGGAAATGTGTCCAACAAAC | SEQ ID NO: 1 |
| SFN-R | GCCACCAATTCCCTGAAACTC | SEQ ID NO: 2 |
| ACTB-F | AATCGCGTGCGCCGTTC | SEQ ID NO: 3 |
| ACTB-R | ATCGGCAAAGGCGAGGCTCT | SEQ ID NO: 4 |
| APAF1-F | GCGCCTTCCACTGCGATATT | SEQ ID NO: 5 |
| APAF1-R | GTTCCCACCAATGCCGGACTC | SEQ ID NO: 6 |
| BRCA1-F | CTGAGAGGCTGCTGCTTAG | SEQ ID NO: 7 |
| BRCA1-R | GAATACCCATCTGTCAGCTTC | SEQ ID NO: 8 |
| CALCA-F | TGCGGAGAGCGAGTCTTAGATAC | SEQ ID NO: 9 |
| CALCA-R | CCAATTACGCGTGACCTCAAC | SEQ ID NO: 10 |
| CASP8-F | CGGCTGGTGAGCAGGAAG | SEQ ID NO: 11 |
| CASP8-R | GCATCTGAGCTCC | SEQ ID NO: 12 |
| | AAGTCCACTCTG | |
| CCND2-F | GACCGTGCTGGCGGACTTC | SEQ ID NO: 13 |
| CCND2-R | TGGCCACACCGATGCAGCTT | SEQ ID NO: 14 |
| DAPK1-F | AGGATCTGGAGCGAACTG | SEQ ID NO: 15 |
| DAPK1-R | GGCTCCGGAAGTGACTG | SEQ ID NO: 16 |
| CDH1-F | CTCCAGCTTGGGTGAAAGAG | SEQ ID NO: 17 |
| CDH1-R | CGTACCGCTGATTGGCTGAG | SEQ ID NO: 18 |
| EDNRB-F | GAGAGGGCATCAGGAAGGAG | SEQ ID NO: 19 |
| EDNRB-R | AGGCCGCAGGCAAGAACCAG | SEQ ID NO: 20 |
| EP300-F | AGGAGGTGAGTGTCTCTTGTC | SEQ ID NO: 21 |
| EP300-R | CTGGAGAGGGATGCGGACTCG | SEQ ID NO: 22 |
| ESR1-A-F | GGTGCCCTACTACCTGGAG | SEQ ID NO: 23 |
| ESR1-A-R | CCGGCGAGAGAACTTGAC | SEQ ID NO: 24 |
| ESR1-B-F | CTCTGGCTGTGCCACACTG | SEQ ID NO: 25 |
| ESR1-B-R | GCACAAAGAATCCTACAAGTC | SEQ ID NO: 26 |
| Fas-F | AATGCCCATTTGTGCAACGA | SEQ ID NO: 27 |
| Fas-R | CGTACTGAGCGGGTCCAC | SEQ ID NO: 28 |
| FHIT-F | GTGCGGTACAGCCTTTCGTTA | SEQ ID NO: 29 |
| FHIT-R | TCCTGTGACCGGACAGAGC | SEQ ID NO: 30 |
| GPC3-F | AGTGGCCCTGAGGAGCAAGAG | SEQ ID NO: 31 |
| GPC3-R | CCAGAGCGCCCTGTGTAGAG | SEQ ID NO: 32 |
| NR3C1-F | GCGTCACCAACAGGTTGCATC | SEQ ID NO: 33 |
| NR3C1-R | TCTCCTTCCACCCACAGAAT | SEQ ID NO: 34 |
| GSTP1-F | TCCGGGATCGCAGCGGTC | SEQ ID NO: 35 |
| GSTP1-R | CGAAGACTGCGGCGGCGAAA | SEQ ID NO: 36 |
| HIC1-F | GTAAAGTTCTCCGCCCTGAATG | SEQ ID NO: 37 |
| HIC1-R | CCGGACCAGGAGAAGGAG | SEQ ID NO: 38 |
| SCGB3A1-F | ACGTTGCCACGGTCTGGGAT | SEQ ID NO: 39 |
| SCGB3A1-R | CAGGCAGGCCCGGCCTTTG | SEQ ID NO: 40 |
| MLH1-F | CGCCACATACCGCTCGTAG | SEQ ID NO: 41 |
| MLH1-R | GCTGTCCGCTCTTCCTATTG | SEQ ID NO: 42 |
| ICAM1-F | CTTAGCGCGGTGTAGACCGT | SEQ ID NO: 43 |
| ICAM1-R | GAGCCATAGCGAGGCTGAG | SEQ ID NO: 44 |
| DNAJC15-F | CATGGCTGCCCGTGGTGTC | SEQ ID NO: 45 |
| DNAJC15-R | GGCGTCAAAGCCCAGCAC | SEQ ID NO: 46 |
| MCTS1-F | AAGTCCCGCCCTTTCAGCTAC | SEQ ID NO: 47 |
| MCTS1-R | ATAGGGAAGGGCCCGGAATG | SEQ ID NO: 48 |
| FABP3-F | GCCACCAGGCAGTGAGAGTGA | SEQ ID NO: 49 |
| FABP3-R | GGCCTCTAGGCACTCTGGAATC | SEQ ID NO: 50 |
| ABCB1-F | TCCACTAAAGTCGGAGTATC | SEQ ID NO: 51 |
| ABCB1-R | TGGTCCAGTGCCACTAC | SEQ ID NO: 52 |
| MGMT-F | ACGGGCCATTTGGCAAAC | SEQ ID NO: 53 |
| MGMT-R | GTCGGCGCATGCCCAGTG | SEQ ID NO: 54 |
| MSH2-F | CTTCCGGGCACATTACGAG | SEQ ID NO: 55 |
| MSH2-R | CACACCCACTAAGCTGTTTC | SEQ ID NO: 56 |
| MUC2-F | CAGGGCTGCCTCATCCTG | SEQ ID NO: 57 |
| MUC2-R | CTCCCAGACGCGACTTG | SEQ ID NO: 58 |
| MYOD1-F | GTTGTTGCACTCGTGCGTTTC | SEQ ID NO: 59 |
| MYOD1-R | CGGCACGCCCTTTCCAAAC | SEQ ID NO: 60 |
| CDKN2B-F | CTGGCCTCCCGGCGATCAC | SEQ ID NO: 61 |
| CDKN2B-R | CATTACCCTCCCGTCGTCCTTC | SEQ ID NO: 62 |
| CDKN2A-F | AGCATGGAGCCTTCGGCTGAC | SEQ ID NO: 63 |
| CDKN2A-R | TCCGGAGAATCGAAGCGCTAC | SEQ ID NO: 64 |
| CDKN1A-F | TGGAGAGTGCCAACTCATTC | SEQ ID NO: 65 |
| CDKN1A-R | TCAGCGCGGCCCTGATATAC | SEQ ID NO: 66 |
| CDKN1B-F | CTCCGAGGCCAGCCAGAG | SEQ ID NO: 67 |
| CDKN1B-R | GGTGGAAGGGAGGCTGACGAAG | SEQ ID NO: 68 |
| CDKN1C-F | ATCGCCGTGGTGTTGTTG | SEQ ID NO: 69 |
| CDKN1C-R | CTGTCCGGTGGTGGACTCT | SEQ ID NO: 70 |
| TP73-F | AAAGGCGGCGGGAAGGAG | SEQ ID NO: 71 |
| TP73-R | CGGCCCCTAGGCGGGTTA | SEQ ID NO: 72 |
| PAX5-F | AAACCCGGCCTGCGCTCG | SEQ ID NO: 73 |
| PAX5-R | CTAGCCAGCGCACCTACG | SEQ ID NO: 74 |
| PGK1-F | CTAAGTCGGGAAGGTTCCTTG | SEQ ID NO: 75 |
| PGK1-R | GCTTGCAGAATGCGGAACAC | SEQ ID NO: 76 |
| PGR-p-F | TCGGCCATACCTATCTCCCT | SEQ ID NO: 77 |
| PGR-p-R | AGCCGGTGGATCTTCGGGA | SEQ ID NO: 78 |
| PGR-d-F | AGTACTCTGCGTCTCCAGTC | SEQ ID NO: 79 |
| PGR-d-R | CAGAGGGAGGAGAAAGTG | SEQ ID NO: 80 |
| RARB-F | GTTTAGGGCTTGCATGTG | SEQ ID NO: 81 |
| RARB-R | CACCAACTCCCAGGATTC | SEQ ID NO: 82 |
| RASSF1-F | CGCGGCTCTCCTCAGCTCCT | SEQ ID NO: 83 |
| RASSF1-R | CCCAGATGAAGTCGCCACAG | SEQ ID NO: 84 |
| RB1-F | CCACAGTCACCCACCAGACTC | SEQ ID NO: 85 |
| RB1-R | TCCTCTCCCGACTCCCGTTA | SEQ ID NO: 86 |
| SLC19A1-F | GATCCAGCTTGCGCCAGGAATG | SEQ ID NO: 87 |
| SLC19A1-R | CGTCCCGCGAACGCGTC | SEQ ID NO: 88 |
| PRDM2-F | CTAGGGTGCGGTCGGACTTG | SEQ ID NO: 89 |
| PRDM2-R | GCCGCCATCTTGACTCCAG | SEQ ID NO: 90 |
| RPL15-F | GCGGTGCGTGAAACAAACCTG | SEQ ID NO: 91 |
| RPL15-R | CCCAGAGCGTCATGGGACATGTAG | SEQ ID NO: 92 |
| S100A2-F | GGGTTGGATTTCAGCAGGATAG | SEQ ID NO: 93 |
| S100A2-R | CAGGGAAGGGAACACCACATAC | SEQ ID NO: 94 |
| SOCS1-F | CACCTGTGCCTGCTAGAAGAG | SEQ ID NO: 95 |
| SOCS1-R | CCTGCGCCAGTCTTTTAAACCG | SEQ ID NO: 96 |
| PRKCDBP-F | TTGCCGTGCCAACACAGTC | SEQ ID NO: 97 |
| PRKCDBP-R | CTTGAAAGCGTTTCGCCTTCCG | SEQ ID NO: 98 |
| SYK-F | CGGGCGCGTTAAGGAAGTT | SEQ ID NO: 99 |
| SYK-R | CCCGTAACCTCCTCTCCTTACC | SEQ ID NO: 100 |
| THBS1-F | AAACGGGCCCAGTCTCTAGT | SEQ ID NO: 101 |
| THBS1-R | CGCGCAACTTTCCAGCTAGA | SEQ ID NO: 102 |
| TES-F | ACGCCCAGAGAATCCCTTCG | SEQ ID NO: 103 |
| TES-R | GCGCCGCTCAACAGCCACTC | SEQ ID NO: 104 |
| PYCARD-F | TGGAATTGAGGGAGCTTCAC | SEQ ID NO: 105 |
| PYCARD-R | AAGGCGCTTCCTTACTACAC | SEQ ID NO: 106 |
| TNFSF11-F | CTCTTGGACCTCCAGAAAGACAG | SEQ ID NO: 107 |
| TNFSF11-R | CTTGGAGCCCGGCTTTGG | SEQ ID NO: 108 |
| PLAU-F | TTCTGTCTGTGCTTCTTGGGAGAG | SEQ ID NO: 109 |
| PLAU-R | CCGCAACGCTCACAAAGATTTGG | SEQ ID NO: 110 |
| VHL-F | CTATTTCCGCGAGCGCGTTC | SEQ ID NO: 111 |
| VHL-R | ATTCCCTCCGCGATCCAGAC | SEQ ID NO: 112 |

TABLE 3

| Gene | Primer 5′ to 3′ | SEQ ID NO |
|---|---|---|
| SFN-F | GGGCTGGAGCTTC | SEQ ID |
| | AGAGGCTGCTTG | NO: 112 |
| SFN-R | GGCCTCTGACCTATG | SEQ ID |
| | AGCTCCAGACTGTG | NO: 113 |
| ACTB-F | AATCGCGTGCGC | SEQ ID |
| | CGTTCCGAAAG | NO: 114 |
| ACTB-R | ATCGGCAAAGGC | SEQ ID |
| | GAGGCTCTGTG | NO: 115 |
| APAF1-R | GCGCCTTCCACT | SEQ ID |
| | GCGATATTGCTC | NO: 116 |
| APAF1-R | GTTCCCACCAA | SEQ ID |
| | TGCCGGACTCG | NO: 117 |
| BRCA1-F | CTGAGAGGCTGCT | SEQ ID |
| | GCTTAGCGGTAG | NO: 118 |
| BRCA1-R | GAATACCCATCTGT | SEQ ID |
| | CAGCTTCGGAAATC | NO: 119 |
| CALCA-F | TGCGGAGAGCGAGT | SEQ ID |
| | CTTAGATACCCAG | NO: 120 |
| CALCA-R | CCAATTACGCGTG | SEQ ID |
| | ACCTCAACAGCTC | NO: 121 |
| CASP8-F | CCGCTGGGAGGC | SEQ ID |
| | TGCCAAAGTTC | NO: 122 |
| CASP8-R | GCATCTGAGCTCCA | SEQ ID |
| | AGTCCACTCTGTTC | NO: 123 |
| CCND2-F | GACCGTGCTGG | SEQ ID |
| | CGGACTTCACC | NO: 124 |
| CCND2-R | TGGCCACACCGA | SEQ ID |
| | TGCAGCTTTCTA | NO: 125 |
| DAPK1-F | GGAGAGGGAGTC | SEQ ID |
| | GCCAGGAATGTG | NO: 126 |
| DAPK1-R | CAGGGACGCCGC | SEQ ID |
| | GGAAGAATGAAG | NO: 127 |
| CDH1-F | CTCCAGCTTGGGT | SEQ ID |
| | GAAAGAGTGAGAC | NO: 128 |
| CDH1-R | CGTACCGCTGATT | SEQ ID |
| | GGCTGAGGGTTC | NO: 129 |
| EDNRB-F | GAGAGGGCATCAGG | SEQ ID |
| | AAGGAGTTTCGAC | NO: 130 |
| EDNRB-R | GCAGGCAAGAAC | SEQ ID |
| | CAGCGCAACCAG | NO: 131 |
| EP300-F | TCTCTTGTCGCC | SEQ ID |
| | TCCTCCTCTCCC | NO: 132 |
| EP300-R | CTGGAGAGGGATG | SEQ ID |
| | CGGACTCGATAG | NO: 133 |
| ESR1-A-F | GGTGCCCTACTAC | SEQ ID |
| | CTGGAGAACGAG | NO: 134 |
| ESR1-A-R | CCGGCGAGAGAAC | SEQ ID |
| | TTGACTCTGAAC | NO: 135 |
| ESR1-B-F | CCACACTGCTCC | SEQ ID |
| | CTGTGAGCAGAC | NO: 136 |
| ESR1-B-R | CCCATGGAGAACA | SEQ ID |
| | GCAATCCTCATC | NO: 137 |
| Fas-F | AATGCCCATTTG | SEQ ID |
| | TGCAACGAACC | NO: 138 |
| Fas-R | CGTACTGAGCG | SEQ ID |
| | GGTCCACCAAC | NO: 139 |
| FHIT-F | GTGCGGTACAGC | SEQ ID |
| | CTTTCGTTACAC | NO: 140 |
| FHIT-R | TCCTGTGACCGG | SEQ ID |
| | ACAGAGCAGAGC | NO: 141 |
| GPC3-F | AGTGGCCCTGAG | SEQ ID |
| | GAGCAAGAGACG | NO: 142 |
| GPC3-R | CACCCTCCTCTC | SEQ ID |
| | GCACTGCCTTCG | NO: 143 |
| NR3C1-F | GCGTCACCAACAG | SEQ ID |
| | GTTGCATCGTTC | NO: 144 |
| NR3C1-R | TCTCCTTCCAC | SEQ ID |
| | CCACAGAATCC | NO: 145 |
| GSTP1-F | TCCGGGATCGCA | SEQ ID |
| | GCGGTCTTAGG | NO: 146 |
| GSTP1-R | CGAAGACTGCGG | SEQ ID |
| | CGGCGAAACTC | NO: 147 |
| HIC1-F | GGTAAAGTTCTCC | SEQ ID |
| | GCCCTGAATGAC | NO: 148 |
| HIC1-R | GGACCAGGAGAAGG | SEQ ID |
| | AGCAGGAGGTGAG | NO: 149 |
| SCGB3A1-F | ACGTTGCCACGGT | SEQ ID |
| | CTGGGATCAGAG | NO: 150 |
| SCGB3A1-R | CAGGCAGGCCCG | SEQ ID |
| | GCCTTTGTCTC | NO: 151 |
| MLH1-F | CGCCACATACCGC | SEQ ID |
| | TCGTAGTATTCG | NO: 152 |
| MLH1-R | GCTGTCCGCTCTTC | SEQ ID |
| | CTATTGGTTCGTTT | NO: 153 |
| ICAM1-F | CTTAGCGCGGTG | SEQ ID |
| | TAGACCGTGATT | NO: 154 |
| ICAM1-R | GAGCCATAGCGA | SEQ ID |
| | GGCTGAGGTTG | NO: 155 |
| DNAJC15-F | CATGGCTGCCCG | SEQ ID |
| | TGGTGTCATCG | NO: 156 |
| DNAJC15-R | GGCGTCAAAGCC | SEQ ID |
| | CAGCACAAAGC | NO: 157 |
| MCTS1-F | AAGTCCCGCCCT | SEQ ID |
| | TTCAGCTACCTC | NO: 158 |
| MCTS1-R | ATAGGGAAGGGCC | SEQ ID |
| | CGGAATGGGAAAG | NO: 159 |
| FABP3-F | GCCACCAGGCAG | SEQ ID |
| | TGAGAGTGAAGG | NO: 160 |
| FABP3-R | TGGCCTCTAGGCA | SEQ ID |
| | CTCTGGAATCTG | NO: 161 |
| ABCB1-F | TTTCACGTCTTG | SEQ ID |
| | GTGGCCGTTCC | NO: 162 |
| ABCB1-R | TGGTCCAGTGCC | SEQ ID |
| | ACTACGGTTTG | NO: 163 |
| MGMT-F | ACGGGCCATTTG | SEQ ID |
| | GCAAACTAAGG | NO: 164 |
| MGMT-R | GGCCTGAGGCA | SEQ ID |
| | GTCTGCGCATC | NO: 165 |
| MSH2-F | CCTGGTGGCAACC | SEQ ID |
| | TACCCTTGCATAC | NO: 166 |
| MSH2-R | AGTCAGCTTCCAG | SEQ ID |
| | GGCTGCGTTTCG | NO: 167 |
| MUC2-F | CAGGGCTGCCTC | SEQ ID |
| | ATCCTGAAGAAG | NO: 168 |
| MUC2-R | CCAAAGACAGGG | SEQ ID |
| | CCAGGCACACAG | NO: 169 |
| MYOD1-F | GTTGTTGCACTCG | SEQ ID |
| | TGCGTTTCTCTG | NO: 170 |
| MYOD1-R | CGGCACGCCCTT | SEQ ID |
| | TCCAAACCTCTC | NO: 171 |
| CDKN2B-F | ACGGAATTCTTT | SEQ ID |
| | GCCGGCTGGCTC | NO: 172 |
| CDKN2B-R | CATTACCCTCCCG | SEQ ID |
| | TCGTCCTICTGC | NO: 173 |
| CDKN2A-F | AGCATGGAGCCT | SEQ ID |
| | TCGGCTGACTGG | NO: 174 |
| CDKN2A-R | TCCGGAGAATCGAA | SEQ ID |
| | GCGCTACCTGATTC | NO: 175 |
| CDKN1A-F | GGGAAATGTGTC | SEQ ID |
| | CAGCGCACCAAC | NO: 176 |
| CDKN1A-R | TCAGCGCGGCCC | SEQ ID |
| | TGATATACAACC | NO: 177 |
| CDKN1B-F | CTCCGAGGCCAGC | SEQ ID |
| | CAGAGCAGGTTTG | NO: 178 |
| CDKN1B-R | GGTGGAAGGGAGG | SEQ ID |
| | CTGACGAAGAAG | NO: 179 |
| CDKN1C-F | ATCGCCGTGGTGTT | SEQ ID |
| | GTTGAAACTGAAA | NO: 180 |
| CDKN1C-R | GGTGGTGGACTCTT | SEQ ID |
| | CTGCGTCGGGTTC | NO: 181 |
| TP73-F | GAGCGCCGGGA | SEQ ID |
| | GGAGACCTTG | NO: 182 |
| TP73-R | CGGCCCCTAGGC | SEQ ID |
| | GGGTTATATGG | NO: 183 |
| PAX5-F | AAACCCGGCCTG | SEQ ID |
| | CGCTCGTCTAAG | NO: 184 |
| PAX5-R | CTAGCCAGCGCA | SEQ ID |
| | CCTACGGGAAG | NO: 185 |
| PGK1-F | CTAAGTCGGGAAGG | SEQ ID |
| | TTCCTTGCGGTTCG | NO: 186 |
| PGK1-R | CGGGCAGGAACAG | SEQ ID |
| | GGCCCACACTAC | NO: 187 |
| PGR-p-F | TCGGCCATACCTA | SEQ ID |
| | TCTCCCTGGACG | NO: 188 |
| PGR-p-R | AGCCGGTGGATCT | SEQ ID |
| | TCGGGAAGTTCG | NO: 189 |
| PGR-d-F | TGCGTCTCCAGTC | SEQ ID |
| | CTCGGACAGAAG | NO: 190 |
| PGR-d-R | CCTGCCCTTGGCC | SEQ ID |
| | TCCATCCTGTCGT | NO: 191 |
| RARB-F | ACAGACAGAAAG | SEQ ID |
| | GCGCACAGAGG | NO: 192 |
| RARB-R | CACCAACTCCCA | SEQ ID |
| | GGATTCTCACAG | NO: 193 |
| RASSF1-F | CGCGGCTCTCC | SEQ ID |
| | TCAGCTCCTTC | NO: 194 |
| RASSF1-R | CCCAGATGAAGTC | SEQ ID |
| | GCCACAGAGGTC | NO: 195 |
| RB1-F | CCACAGTCACCCA | SEQ ID |
| | CCAGACTCTTTG | NO: 196 |
| RB1-R | TCCTCTCCCGACT | SEQ ID |
| | CCCGTTACAAAA | NO: 197 |
| SLC19A1-F | GATCCAGCTTGCG | SEQ ID |
| | CCAGGAATGCAG | NO: 198 |
| SLC19A1-R | GTCCCGCGAA | SEQ ID |
| | CGCGTCCTGA | NO: 199 |
| PRDM2-F | CTAGGGTGCGG | SEQ ID |
| | TCGGACTTGCC | NO: 200 |
| PRDM2-R | GCCGCCATCTTGA | SEQ ID |
| | CTCCAGTCGGAA | NO: 201 |
| RPL15-F | GCGGTGCGTGAAA | SEQ ID |
| | CAAACCTGTTCTC | NO: 202 |
| RPL15-R | CCCAGAGCGTCATG | SEQ ID |
| | GGACATGTAGTTC | NO: 203 |
| S100A2-F | GGCATGGGCATGT | SEQ ID |
| | GTGGGCACGTTC | NO: 204 |
| S100A2-R | CCACATACCAGGGC | SEQ ID |
| | CTGTGGGCAGTTG | NO: 205 |
| SOCS1-F | CACCTGTGCCTGCT | SEQ ID |
| | AGAAGAGTCTCATC | NO: 206 |
| SOCS1-R | CCTGCGCCAGTCT | SEQ ID |
| | TTTAAACCGGCTC | NO: 207 |
| PRKCDBP-F | TTGCCGTGCCAA | SEQ ID |
| | CACAGTCTCTGC | NO: 208 |
| PRKCDBP-R | CTTGAAAGCGTTTC | SEQ ID |
| | GCCTTCCGCTGTC | NO: 209 |
| SYK-F | CGGGCGCGTTAA | SEQ ID |
| | GGAAGTTGCCCA | NO: 210 |
| SYK-R | CCCGTAACCTCCT | SEQ ID |
| | CTCCTTACCAGAA | NO: 211 |
| THBS1-F | AAACGGGCCCAGT | SEQ ID |
| | CTCTAGTATCCAC | NO: 212 |
| THBS1-R | GCGCGCAACTTTC | SEQ ID |
| | CAGCTAGAAAGTG | NO: 213 |
| TES-F | ACGCCCAGAGAA | SEQ ID |
| | TCCCTTCGGAG | NO: 214 |
| TES-R | CGAACACGGGAA | SEQ ID |
| | ACCTGCGGAAC | NO: 215 |
| PYCARD-F | TGGAATTGAGGGAG | SEQ ID |
| | CTTCACGCTTCTA | NO: 216 |
| PYCARD-R | AAGGCGCTTCCTTA | SEQ ID |
| | CTACACCCTTGGTC | NO: 217 |
| TNFSF11-F | GGACCTCCAGAAAG | SEQ ID |
| | ACAGCTGAGGATG | NO: 218 |
| TNFSF11-R | CTTGGAGCCCGG | SEQ ID |
| | CTTTGGGTCCTG | NO: 219 |
| PLAU-F | GTCGCGTGATGAAG | SEQ ID |
| | ACTTCACAGCTCC | NO: 220 |
| PLAU-R | CCCAACAGCGTCT | SEQ ID |
| | GGACTGAGGAATC | NO: 221 |
| VHL-F | CTATTTCCGCGA | SEQ ID |
| | GCGCGTTCCATC | NO: 222 |
| VHL-R | ATTCCCTCCGCG | SEQ ID |
| | ATCCAGACCACC | NO: 223 |

5. Development and Manufacture of the Array

Oligonucleotide arrays are custom designed by Microarrays, Inc (Nashville, Tenn.). Probes for the array are 50-60 mers to keep hybridization and washing temperatures high (Relogio et al., 2002, Nucleic Acids Res 30:e51). Probes have been designed according to the Affymetrix model (Mei et al., 2003, Proc. Natl. Acad. Sci. 10:11237-11242). Three types of control probes are present on the array: (1) transcribed regions from Arabidopsis thaliana (definitive negative control, heterologous); (2) transcribed regions of human α-tubulin, β-actin and glyceraldehyde-phosphate-dehydrogenase (GAPDH, definitive negative controls, homologous); (3) promoters of β-actin, phosphoglycerate kinase (PGK1) and ribosomal protein L15 (conditional homologous negative control). HPLC-purified oligonucleotides with an amino group and a six-carbon spacer at the 5′-end are spotted on aminosilane-modified glass slides in triplicate, so each slide contains three identical subarrays. Attachment of the probe is done by incubation at 60° C. for 3.5 hr and for 10 min at 120° C. Slides are stored under vacuum in the dark at room temperature. Genes to be tested in the DNA methylation assay include those listed in Table 1 that are specific to the cancer diagnostic being performed, as shown in Figures. These genes represent different functional groups; all of them have been identified as methylated in different types of cancer. This project will be the first to test methylation of all of them in the same sample of normal ovarian tissue and ovarian cancer.

6. Probe Hybridizations with Microarray

Competitive hybridization of the PCR probes to oligonucleotide arrays is done in rotating tubes in the hybridization chamber. The slides are pre-hybridized for 1 hr at 42° C. in 5×SSC, 0.1% SDS, 1% BSA, rinsed with deionized water and dried by short centrifugation. Hybridization space is created on the slide by Microarray GeneFrames (AbGene, Rochester, N.Y.). Denatured DNA is added to the array, the coverslip is sealed, and the slides are incubated in the dark at 42° C. for 18 hr. After hybridization the GeneFrame and the coverslip are removed, and the slides are washed with shaking in a set of buffers heated to 42° C.: 5 min in 1×SSC, 0.1% SDS; 5 min in 0.1×SSC, 0.1% SDS; 3 min in 0.1×SSC, 0.1% SDS. Slides are dried by a short, low-speed centrifugation and stored in the dark before scanning.

During optimization of the procedure, a single PCR product was labeled with two different fluorophores, probes were mixed, and used for hybridization. In this mixture Cy5- and Cy3-labeled fragments were represented equally imitating conditions for methylated fragments. Mean Cy5/Cy3 ratio calculated from such experiments produced the normalization coefficient to account for fluorophore-related differences in labeling and detection.

7. Signal Detection and Sample Scoring

Scanning is done with ScanArray™ 4000XL (Packard BioChip) according to the manual. ScanArray™ software allows selection of different Photo Multiplier Tube (PMT) gain parameters to adjust to different quantum yields of Cy3 and Cy5 fluorophores; these parameters were established experimentally based on the maximum signal strength and minimum background/PMT noise. The protocol (EasyScan) for detection of two fluorophore hybridizations is used.

Quantitation of the signal is done using the Adaptive Circle algorithm of the ScanArray™ software. Initially the signals are normalized to account for differences in fluorophore incorporation and detection. The percentage of the signal for an individual spot relative to the total signal from the corresponding fluorophore is used to normalize signals across the array and then the ratio of the Cy5/Cy3 percentages for each spot is computed. An alternative technique makes use of the expected distribution of the ratios and allows for differences in methylation status at the majority of sites under investigation. Suppose we observe (xᵢ,yᵢ), i=1, . . . , n where xᵢ is the Cy3 intensity and yᵢ is the Cy5 intensity for specimen i. The goal of normalization is to find a function, ƒ(.) such that yᵢ≧ƒ(xᵢ), for most of the regions. A smoothed lower boundary for the cloud (xᵢ,yᵢ), i=1, . . . , n can be achieved by non-parametric quantile regression in which the 10-20% quantile curve is used as the normalizing function ƒ(.). Such a function will allow measurement error so that some yᵢ values may be slightly less than ƒ(xᵢ). In the end, the ratio rᵢ=yᵢ/ƒ(xᵢ) is then used to measure the signal. This technique will produce ratios that are either close to 1 or >1 and will reduce the number of methylation sites with middle range ratios (1.3 to 2). After the signals are normalized, ratios will be computed.

The percentage normalization method allows the detection of very high Cy3:Cy5 ratios (up to 5,000) and approximately equal ratios (between 0.8 and 1.2), which correspond to unmethylated and methylated sites, respectively. Some genes fall in the intermediate range (genes methylated in some part of the population with ratios between 1.3 and 2) and are removed from the diagnostic set. The quantile regression normalization method eliminates these intermediate values, so no manual adjustment is required.

The pattern of expression microarray analysis is followed and non-specific filtering is applied to remove uninvolved or uninformative features from consideration before selecting the most divergent in their methylation status (Scholtens and von Heydebreck, 2005, Studies is Bioinformatics and Computational Biology Solutions using R and Bioconductor, Gentle, am et al., Eds.). Two non-specific filters are applied: 1) for all samples investigated, 80% of the samples must give interpretable ratios (<1.3 or >2); and 2) at least 10% differential methylation must be observed across all samples (e.g., 90% methylated and 10% unmethylated). After the non-specific filtering step, methylation sites (features) are selected on the basis of differential status in the cancer and normal tissues. For feature selection and classifier design the Support Vector Machine algorithm is used, which has been developed for pattern recognition tasks (Model et al., 2001, Bioinformatics 17(Suppl. 1):S157-164). All samples are divided into a training set and a test set. Initially, Support Vector Machine is used with the training set to select features and create the classifier function, which is then validated with a “leave-one-out” analysis using the same training set (Lee et al., 2004, IEEE Trans. Neural. Netw. 15:750-757). Results are subsequently evaluated using the Fisher's Exact test.

### B. Results

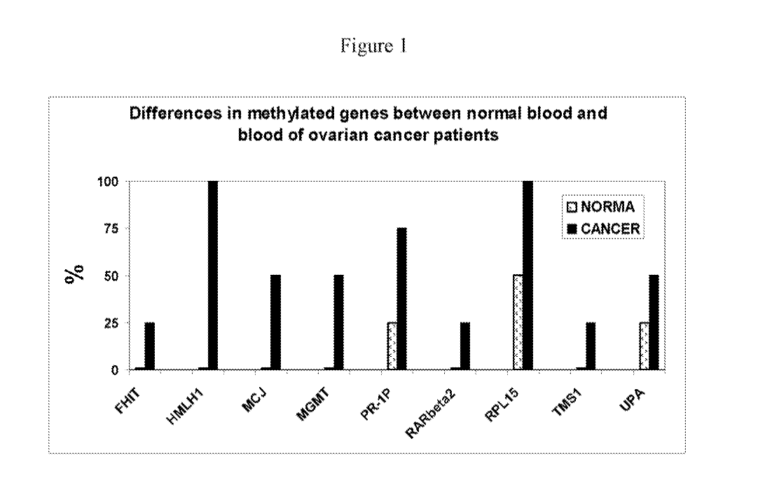

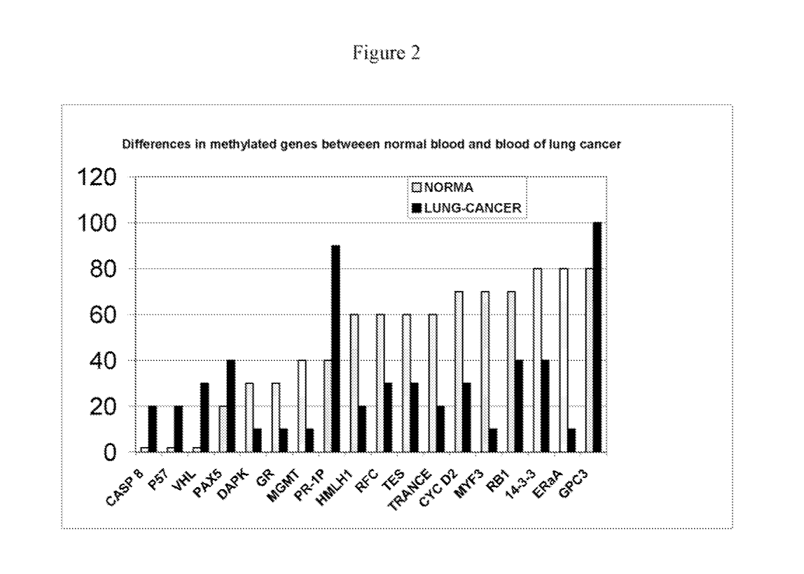

Ovarian cancer methylation profiling is seen in FIG. 1. Genes studied include FHIT, MLH1, DNAJC15, MGMT, progesterone receptor (e.g., PR-1P or PR-2D), RARB, RPL15, PYCARD and PLAU. The graph demonstrates the percentage of methylated genes relative to the methylation status of their normal counterpart. The genes studied all showed increased methylation in ovarian cancer as compared to a non-cancerous patient. Such patterns or methylation can be used as diagnostic for ovarian cancer. FIG. 2 shows the methylation profiling in plasma DNA from lung cancer patients. The results show high frequency of CpG island methylation in genes CASP8, CDKN1C, VHL, PAX5, progesterone receptor (e.g., PR-1P or PR-2D) and GPC3 relative to methylation found in DNA from normal subjects.

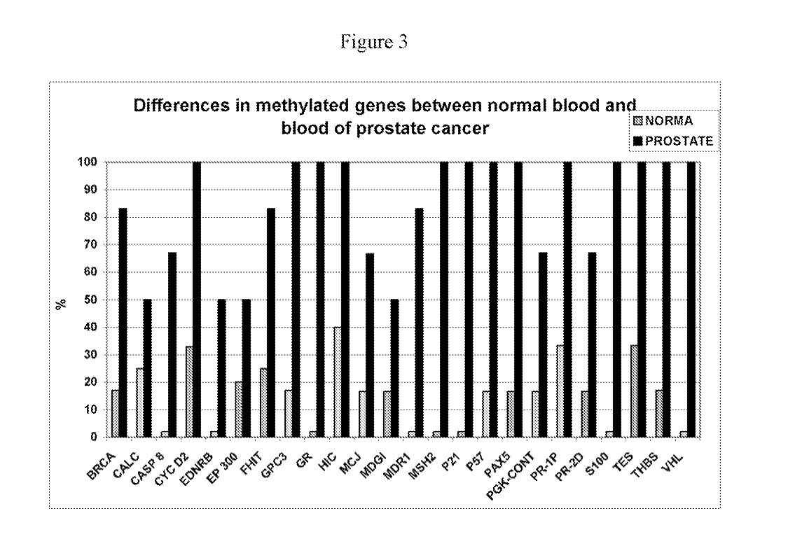

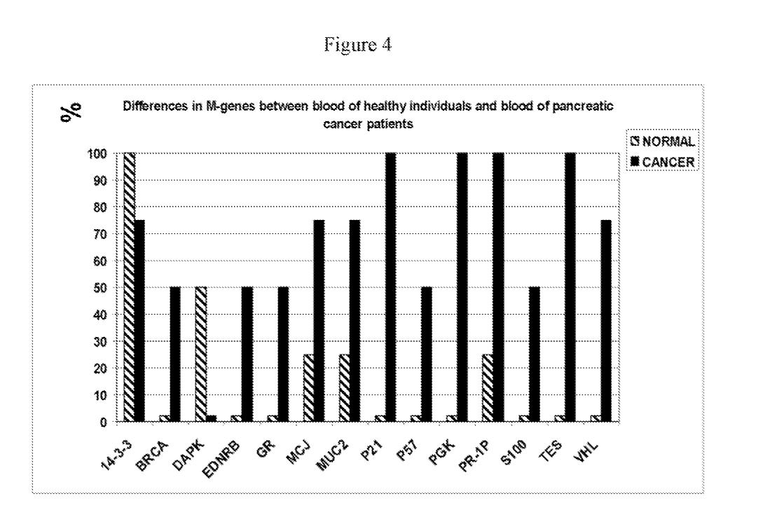

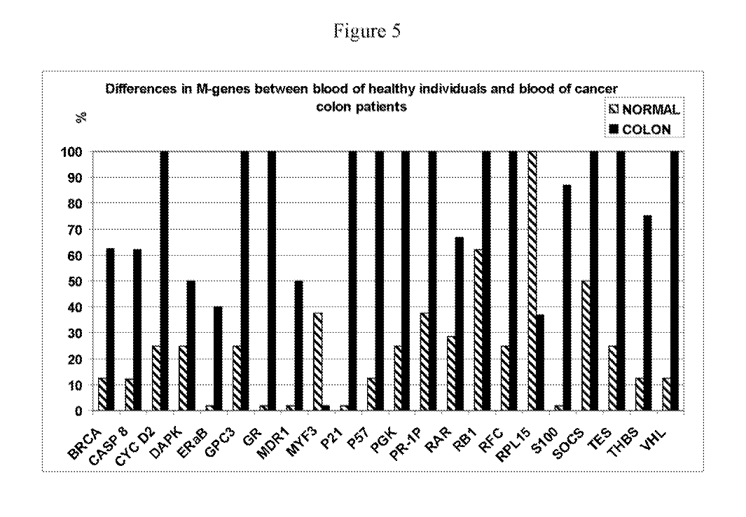

High frequency of methylation is seen in all genes tested in DNA from prostate cancer subjects relative to normal subject DNA, as seen in FIG. 3. However, of the genes tested for methylation in DNA from pancreatic cancer subjects, all but DAPK1 and SFN showed increased CpG methylation in cancer DNA (FIG. 4). When assaying plasma DNA from colon cancer patients, as can be seen in FIG. 5, MYOD1 and RPL15 are the only two genes tested that did not demonstrate increased frequency of CpG methylation over normal.

FIGS. 1-5 all show distinctive gene methylation patterns for various cancers, thereby allowing for profiling, diagnosing, and characterization of the related cancers.

### Example II

### A. Introduction

Early detection of breast cancer improves survival rates and quality of life, so screening for breast cancer is an important target of public health (Knutson D, Steiner E., Am Fam Physician, 75:1660-6 (2007)). Screening by mammography affords early detection, but its sensitivity is influenced by many factors, including tissue density and the stage of the disease (Berg W A, et al. Radiology, 233:830-49 (2004)).

DNA methylation is an attractive paradigm for cancer detection in that differential methylation of multiple genes in normal versus tumor tissue is well-established (Baylin S B, Ohm J E., Nat Rev Cancer, 6:107-16 (2006); Jones P A., Semin Hematol, 42:S3-8 (2005); Feinberg A P, Tycko B., Nat Rev Cancer, 4:143-53 (2004)). Identical modification of DNA in multiple sites allows testing of multiple biomarker candidates by the same technique. While analysis of each separate biomarker may not be adequate for diagnosis, combinations of biomarkers can produce accurate assays for cancer detection. Such assays together with the presence of abnormally methylated DNA in the blood of cancer patients (Taback B, Hoon D S., Acad Sci, 1022:1-8 (2004); Fiegl H, et al., Cancer Res, 65:1141-5 (2005)), create a possibility for a minimally-invasive diagnostic test.

We have developed a platform for multiplex detection of DNA methylation at multiple genomic sites (Melnikov A A, et al., Nucleic Acids Res, 33:e93 (2005)) and tested its performance in DNA from fixed human tissues (Bhandare D J, et al., Clin. Chim. Acta, 367:211-3 (2006)). Here we present proof-of-principle data on selection of informative methylated or unmethylated promoter sequences for cancer detection using DNA from gross sections of formalin-fixed paraffin-embedded (FFPE) clinical specimens. Our approach allows detection of pathological changes via an observer-independent assay, which has obvious advantages for clinical practice.

### B. Materials and Methods

1. Clinical Samples

The project was approved by the Institutional Review Board of Northwestern University. “Infiltrating ductal carcinoma” or “IDC” was defined as malignant mammary epithelial cells invading stroma. Samples of well, moderately and poorly differentiated IDC were examined. Most samples were invasive carcinoma with accompanying DCIS. “Ductal carcinoma in situ” or “DCIS” was defined as malignant mammary epithelial cells contained within ducts or duct-like structures. Samples contained well, moderately and poorly differentiated DCIS, while samples with invasive carcinoma were excluded. “Atypical Ductal Hyperplasia” or “ADH” was defined according to Page and Tavassoli (Jensen R A, et al., J Cell Biochem Suppl, 17G:59-64 (1993); MacGrogan G, Tavassoli F A., Virchows Arch, 443:609-17 (2003)) as lesions having all the characteristics of low grade DCIS but less than 2 mm in size or, if larger lesions, having only some characteristics of DCIS. Samples with papillomas and radial scars with atypical hyperplasia were sometimes present, but those with DCIS and/or IDC or more advanced disease were excluded. Normal breast tissue samples from reduction mammaplasty (diagnosis of macromastia) contained either no pathological changes or the changes were minimal (fibrosis, fibroadenoma).

All samples were collected using IRB-approved protocols, evaluated by a pathologist, and stored as FFPE blocks. They were identified by Surgical Pathology Final Reports (without personal data) and reviewed by one of the authors (ELW). One ten-micron section was used for DNA isolation. There were no attempts to isolate tumor cells or to remove uninvolved areas. The ethnicity of the subjects was not considered. The ages of the subjects and tumor characteristics are presented in data provided in Table 4.

TABLE 4
Characteristics of clinical specimens

| | Tissue type | | | |
|---|---|---|---|---|
| | DCIS | IDC | ADH | Normal |
| | (n = 28) | (n = 39) | (n = 40) | (n = 31) |
| Age | | | | |
| Mean (SD) | 58.8 (11.1) | 52.2 (13.3) | 57.6 (11.6) | 33.2 (10.5) |
| Range | 40-81 | 33-80 | 36-91 | 22-61 |
| p-value† | <0.001 | | | |
| Grade | | | | |
| 1 | 10 | 2 | ND | ND |
| 2 | 9 | 5 | ND | ND |
| 3 | 9 | 32 | ND | ND |
| p-value‡ | <0.001 | | | |
| Estrogen receptor | | | | |
| Fraction positive | 1 | 0.55 | NA | NA |
| Reported value | .64ⁿ¹ | .64ⁿ¹ | NA | NA |
| p-value* | <0.001 | 0.31 | | |
| Progesterone receptor | | | | |
| Fraction positive | 0.75 | 0.5 | NA | NA |
| Reported value | .57ⁿ¹ | .57ⁿ¹ | NA | NA |
| p-value* | 0.06 | 0.42 | | |
| TP53 | | | | |
| Fraction positive | 0.19 | 0.47 | NA | NA |
| Reported value | .185ⁿ² | .53ⁿ³ | NA | NA |
| p-value* | 0.81 | 0.51 | | |

†p-value from ANOVA model of age on tissue type; Bonferroni corrected p-values for pairwise comparisons demonstrate a significant difference in the normal group compared to all others (p < 0.001)
‡p-value from Fisher's Exact Test analog for 3 × 2 table comparing DCIS and IDC grades
*p-value from exact binomial test comparing observed proportions to literature-reported values
ⁿ¹Leonard GD, et al. Breast J, 10:146-9 (2004).
ⁿ²Rajan PB, et al., Breast Cancer Res Treat, 42:283-90 (1997).
ⁿ³Tan P, et al., Oncol Rep, 6:1159-63 (1999).

2. DNA Isolation

After xylene deparaffination and ethanol precipitation; the tissue pellet was processed using a DNeasy Tissue kit (Qiagen, Valencia, Calif.). Purified DNA was dissolved in 10 mM Tris pH 7.8, 0.5 mM EDTA.

3. Microarray Mediated Methylation Assay: Overall Approach

In the microarray mediated methylation assay (M³-assay), one portion of each genomic DNA sample was digested with a methylation-sensitive restriction enzyme while another portion of the same sample served as an undigested control. Selected regions of the genomic DNA from each of the digested and undigested DNA samples were amplified by PCR using gene-specific primers that flank restriction sites. For the amplified product from the digested portion only fragments with methylated sites were capable to serve as templates, whereas in the undigested (control) portion, all fragments were amplified. Comparison between the two sets of PCR products was done by gel electrophoresis (MSRE-PCR) (Melnikov A A, et al., Nucleic Acids Res, 33:e93 (2005)) or by competitive hybridization with custom-designed microarrays (M³-assay). Fluorescent signals of hybridized fragments in the M³-assay were separately scored, and the ratio between the signals from control and digested DNAs was calculated. This ratio was used to assign “methylated” or “unmethylated” calls to the targeted regions. The data were statistically assessed to select groups of informative fragments, which were then analyzed together as a composite biomarker. Details of the method are presented below.

4. Microarray Mediated Methylation Assay: DNA Digestion

Hin6I (Fermentas, Hanover, Md.) was used to digest one half of each purified genomic DNA sample as described (Melnikov A A, et al., Nucleic Acids Res, 33:e93 (2005)). The second half of each DNA sample was incubated in the digestion buffer but without the enzyme and served as the control.

5. PCR Amplification

Nested PCR was performed as described (Melnikov A A, et al., Nucleic Acids Res, 33:e93 (2005)). KlenTaq1 (Barnes W M., Proc Natl Acad Sci USA, 91:2216-20 (1994)) (DNA Polymerase Technology, St. Louis, Mo.) was used at 8 Upper 30 μl reaction. Betain and dNTPs (Sigma, St. Louis, Mo.) were added to the PCR buffer to 1.5M and 0.25 mM, respectively. The PCR reaction was assembled on ice, the tubes were placed into a thermocycler (ABI 9600, Applied Biosystems, Foster City, Calif.), incubated at 95° C. for 5 min, and KlenTaq1 was added. After 25 cycles (95° C.; 45 sec—62° C.; 1 min—72° C.; 1 min) the products were precipitated, dissolved in TE, and 1.5 ng was used for the second PCR, assembled with aminoallyl-dUTP (Biotium, Hayward, Calif.) and dTTP (3:1), and performed as the first. PCR products were precipitated, and dissolved for labeling in 20 μl of 100 mM NaHCO₃ buffer (pH 9.0).

6. DNA Labeling

Five microliters of Cy3 or Cy5 (Monoreactive Dye Pack, Amersham, Piscataway, N.J.) in DMSO were dried in a vacuum and PCR products were added for 2 hrs at room temperature. Unreacted dyes were quenched by 10 μl of 4M hydroxylamine, and the products were precipitated. The PCR products from undigested (control) DNA were labeled with Cy5, while Cy3 was used to label the PCR products from Hin6I-digested DNA.

7. Hybridization and Signal Detection

Custom-designed arrays (MWG Bioinformatics, High Point, N.C.) containing 60-mer probes for each amplified product were printed in triplicate on aminosilane-modified glass by Microarrays, Inc (Nashville, Tenn.). The slides were pre-hybridized for 1 hr at 42° C. in 5×SSC, 0.1% SDS, 1% BSA, rinsed with deionized water and dried. Labeled DNA was dissolved in the hybridization buffer (100 μl; Ocimum Biosolutions, Indianapolis, Ind.), denatured (2 min; 95° C.), and quenched on ice. Microarray GeneFrames (AbGene, Rochester, N.Y.) were used to create space between the slide and the coverslip. Denatured DNA was added, the coverslip was sealed, and the slides were incubated 18 hr at 42° C. The GeneFrame and the coverslip were removed, and the slides were washed at 42° C. for 5 min in 1×SSC, 0.1% SDS; and twice for 5 min in 0.1×SSC, 0.1% SDS. Slides were scanned using ScanArray XL4000 (Perkin Elmer, Boston, Mass.; sensitivity ≦0.1 molecule per μm²) with ScanArray™ software. Intensity of each fluorophor was measured for each spot, and the background values were subtracted. Ratios of Cy5/Cy3 fluorescence were calculated to compare the yields of PCR products from control and Hin6I-digested DNA.

8. Statistical Analysis

Methylation calls were made independently for each spot, and final gene-specific calls were made according to the majority call from the triplicate spots for that gene. Non-specific filtering removed uninformative spots; informative genes were selected by Fisher's Exact Test for differential methylation in each pairwise analysis. Naïve Bayes classification with uninformative prior was used to classify samples assuming that methylation was independent for each of the analyzed sites. The predictive ability of the naïve Bayes classifier for all four pairwise comparisons (cancer v. Normal, IDC v. Normal, DCIS v. Normal, and ADH v. Normal) was evaluated using five-fold cross-validation. The data were partitioned into five sets with equal distribution of each type of specimens. Each set then served as a test set based on training of the naïve Bayes classifier with the other four sets. The number of misclassifications was counted over all five runs and over 25 random partitions of the data into five groups. Gene selection and classifier parameter estimation were performed anew with each round of cross-validation.

9. Assessment of Assay Variability

Methylation profiling of genomic DNA of MCF-7 was repeated five times. Forty nine spots were unambiguously detected and their methylation calls were independently established for each experiment, creating forty nine groups (the number of fragments) of five calls each (five repeats). All calls different from the majority were counted; the number of these calls divided by the total number of calls was used as a measure of the assay's variability.

### C. Results

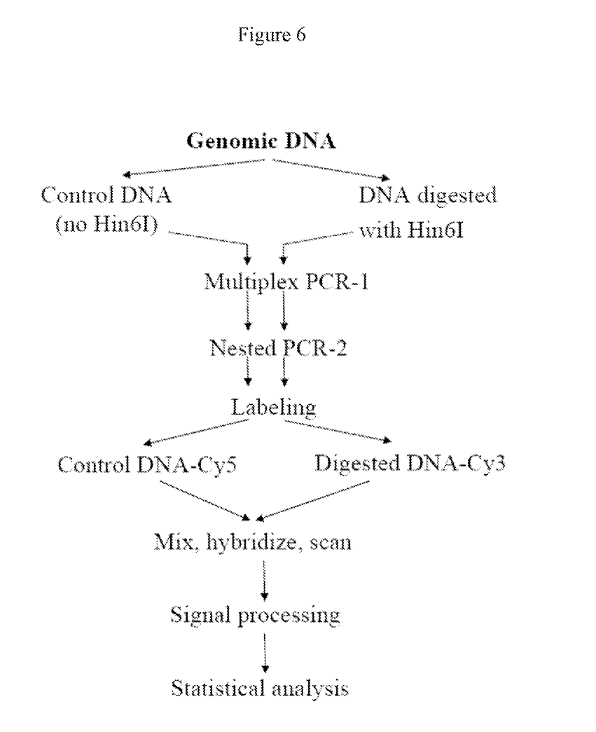

In this project, we evaluated the possibility of observer-independent analysis of heterogeneous clinical samples with the overall goal of identifying DNA fragments informative for cancer detection. DNA methylation signatures were created for each sample using the microarray-mediated methylation assay (M³-assay) developed in our laboratory (FIG. 6). Formalin-fixed paraffin-embedded (FFPE) breast tissues were used.

1. Clinical Samples

The most advanced stage in each sample was used to assign samples to ADH, DCIS and IDC groups, so tumors with IDC could contain regions with DCIS and ADH, while DCIS samples could include regions with ADH. To ensure observer-independent evaluation, we did not microdissect tumor-containing regions.

Age distribution was similar within each group (Table 4). The mean age was lower for reduction mammaplasty (normal) group (p<0.001 using an ANOVA model). The age difference was significant between the normal and other groups (adjusted p-values <0.001 in pairwise comparisons with Bonferroni adjusted p-values). Data on the expression of estrogen and progesterone receptors, and p53 were not available for ADH and normal samples. In DCIS, the fraction of estrogen receptor-positive tumors (100%) was higher than reported (p<0.001), but the fraction of progesterone positive tumors (75%) was similar (Leonard G D, et al., Breast J, 10:146-9 (2004)). In IDC, the fraction of tumors expressing estrogen and progesterone receptors was consistent with reported values (Leonard G D, et al., Breast J, 10:146-9 (2004)). The percentage of p53-positive tumors was close to reported for both DCIS (Rajan P B, et al., Breast Cancer Res Treat, 42:283-90 (1997)) and IDC (Tan P, et al., Oncol Rep, 6:1159-63 (1999)) groups.

2. M³-Assay

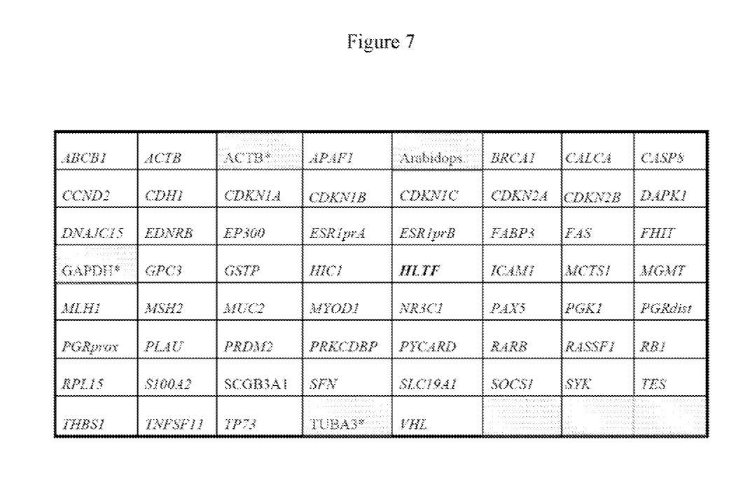

DNA methylation analysis was performed as shown in FIG. 6. Fifty six promoter fragments were interrogated (FIG. 7) in each experiment. Negative control fragments included coding sequences of three genes (marked with * in FIG. 7) and heterologous DNA from A. thaliana. Each probe on the array was designed to detect corresponding PCR product. Each microarray contained three identical sub-arrays, so that every hybridization signal was confirmed in triplicate. Unreliable hybridization signals with intensities comparable to or less than background were excluded, and background was subtracted. The threshold for methylation was determined experimentally using “self-self” hybridizations (Yang Y H, et al., Nucleic Acids Res, 30:e15 (2002)); i.e., PCR products from control (undigested) DNA were divided into two equal aliquots, labeled with either Cy3 or Cy5, mixed and hybridized to the array; the average Cy5/Cy3 ratio was recorded. This “self-self” design assured equal representation of Cy3- and Cy5-labeled fragments as would be expected from samples of methylated DNA. This average ratio of intensities was used as a threshold to define methylation (standard methylation call, SMC). SMCs were used to assign calls for each gene, “methylated (M)”—to genes with Cy5/Cy3≦SMC, and “unmethylated (U)”—to genes with Cy5/Cy3>SMC; an example of data is shown in Table 5. If no call could be assigned, the gene was scored as NA (non-applicable).

TABLE 5
SMC-based call assignment*

| | | | | Methylation |
|---|---|---|---|---|
| Gene | Cy5 | Cy3 | Ratio | Call |
| ABCB1 | 64400 | 64946 | 1.0 | M |
| SFN | 64450 | 64976 | 1.0 | M |
| CDKN2B | 64547 | 63763 | 1.0 | M |
| RPL15 | 64524 | 60570 | 1.1 | M |
| PGK1 | 64510 | 50217 | 1.3 | M |
| FABP3 | 64490 | 40435 | 1.6 | M |
| RASSF1 | 10212 | 6360 | 1.6 | M |
| BRCA1 | 64504 | 36053 | 1.8 | M |
| PAX5 | 64561 | 33619 | 1.9 | M |
| DNAJC15 | 64504 | 32923 | 2.0 | M |
| SLC19A1 | 17732 | 8786 | 2.0 | M |
| EDNRB | 44391 | 17758 | 2.5 | M |
| ESR1 promoter A | 5807 | 2210 | 2.6 | M |
| CDKN1C | 37616 | 13193 | 2.9 | M |
| MCTS1 | 64509 | 17836 | 3.6 | M |
| TNFSF11 | 15402 | 1389 | 11.1 | U |
| CDH1 | 6044 | 508 | 11.9 | U |
| ICAM1 | 51208 | 3997 | 12.8 | U |
| EP300 | 64551 | 4781 | 13.5 | U |
| PGR distal promoter | 61207 | 2653 | 23.1 | U |
| TP73 | 31236 | 1304 | 24.0 | U |
| MGMT | 64423 | 2336 | 27.6 | U |
| MSH2 | 50032 | 534 | 93.8 | U |

*SMC = 4.0

3. Validation of the Assay

A previously validated procedure (MSRE-PCR) (Melnikov A A, et al., Nucleic Acids Res, 33:e93 (2005)) was used for methylation detection. Every assay included two stages: 1) detection of methylation by MSRE digestion, and 2) detection of the signal for each promoter fragment. Briefly, the analytical sensitivity of the assay was determined to be 60 pg for one gene in MSRE-PCR (Bhandare D J, et al., Clin. Chim. Acta, 367:211-3 (2006)) or 100 pg for multiple genes in M³-assay (data not shown). Digestion was confirmed by real-time PCR for selected genes (Melnikov A A, et al., Nucleic Acids Res, 33:e93 (2005)), by detection of unmethylated genes in the M³-assay, and by preservation of methylation patterns in experiments with increased digestion (data not shown). Similar, if not identical, methylation patterns were detected by the MSRE-PCR and bisulfite-based assays (methylation-sensitive PCR and bisulfite sequencing (Melnikov A A, et al., Nucleic Acids Res, 33:e93 (2005))); in addition, comparison of MSRE-PCR data with published results revealed a remarkable degree of correlation (Melnikov A A, et al., Nucleic Acids Res, 33:e93 (2005)).

No attempt was made to correlate the results of the M³-assay and expression profile of analyzed samples. By its design, the M³-assay assessed methylation only in a few CpG sites in each promoter, so a rigorous correlation between gene expression and methylation results could not be expected.

Reproducibility of the M³-assay was evaluated using genomic DNA from MCF-7 cells. The assay was repeated five times, and the readout was evaluated for each fragment as described in Materials and Methods. Six out of 245 total data points were variable (2.4%), suggesting a variability of less than 3% for the assay.

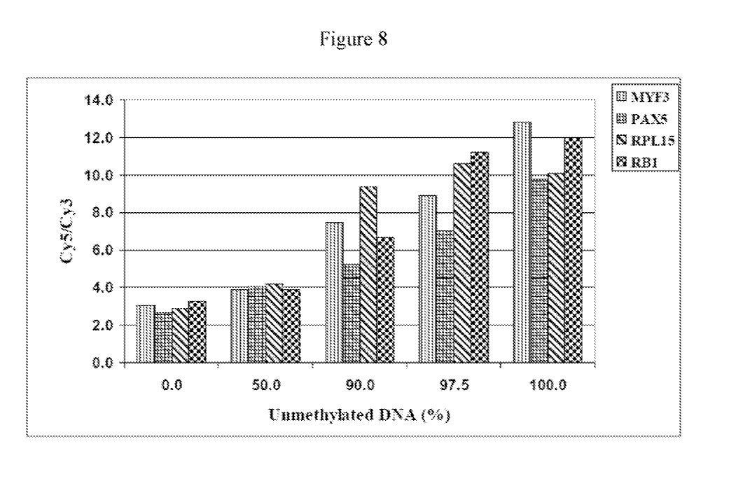

We also evaluated the link between the Cy5/Cy3 ratio and the level of methylation in heterogeneous samples. Control samples were prepared using a mixture of genomic DNA from MCF-7 and TD47D cells so that each sample contained a pre-determined percentage of methylated and unmethylated genes. Cy5/Cy3 ratios below SMC were observed for samples with up to 50% unmethylated DNA (FIG. 8). Samples with greater than 50% unmethylated genomic DNA fragments caused gradual increases of the Cy5/Cy3 ratio (FIG. 8). These results indicate that the efficient detection of methylated fragments incorporated in the MSRE-PCR procedure (Melnikov A A, et al., Nucleic Acids Res, 33:e93 (2005)) was preserved in the M³-assay.

The likelihood of potential PCR bias in the M³-assay was reduced by the use of the same sets of primers and amplification conditions for digested and control DNA, so controllable parameters (DNA concentration, amplicon length, primer concentration, etc) were identical. Each specimen contained multiple genes that produced high signal in digested sample and were scored as “methylated” based on the selected criteria, thus providing direct evidence against such a bias. Each sample also contained several genes that were scored as “unmethylated”, thus providing evidence that Hin6I digestion was efficient.

4. Classification of Samples

Each sub-array contained 61 fragments and three empty spots (FIG. 7) producing 192 spots on the array, 183 of which contained probes. Methylation calls were made in a blinded manner and independently for each spot. The majority call for the three spots for each gene was assigned as a final gene-specific methylation call. If there was no majority, the final call was NA. In a total of 8418 calls made for 61 genes in each of 138 samples, 4725 were M (56.1%), 2045 were U (24.3%), and 1648 (19.6%) were NA.

Similar to expression microarray analysis (Scholtens D, von Heydebreck, A., H. W. Gentleman R, Irizarry R, Dudoit S, Editor. (2005)), non-specific filtering was used to eliminate uninformative genes with detectable calls in less than ⅔ of the samples or less than 10% differential methylation across the entire sample set (e.g. 90% M and 10% U). Non-specific filtering steps were repeated for four pairwise analyses, but only a few genes were eliminated, and over forty-five genes were selected for each comparison: DCIS v. Normal—46 genes; IDC v. Normal—48 genes; DCIS/IDC v. Normal—48 genes; ADH v. Normal—49 genes. Informative features for classifiers were selected with Fisher's Exact test using p<0.10. The moderate p-value of 0.10 was chosen to narrow the set of genes, but to include informative genes with occasionally inflated p-values.

The apparent independence of methylation sites Model F, et al., Bioinformatics, 17 Suppl. 1:S157-164 (2001)) suggested selection of the naïve Bayes classifier (Domingos P, Michael J. Pazzani, Machine Learning, 29:103-130 (1997)), which performed surprisingly well even when independence was not satisfied (Worm J, et al., J Biol Chem, 276:39990-40000 (2001)). Naïve Bayes classifiers were constructed using the e1071 R (R Development Core Team, 2005) package (Gentleman R C, et al., Genome Biol, 5:R80 (2004)), using an uninformative prior with probabilities of 0.5 for each group in the pairwise classification schemes.

Sensitivity and specificity of the assay, and overall classification accuracy was determined (Table 6). Besides DCIS and IDC groups a combined Cancer group was created, which contained both DCIS and IDC samples.

TABLE 6
Performance of M³-assay
True Status

| Predicted | | Cancer | Normal | 1. Cancer classifier* |
|---|---|---|---|---|
| Status | pCancer | 0.7239 | 0.2526 | |
| | pNormal | 0.2761 | 0.7474 | |
| | | ADH | Normal | 2. ADH classifier |
| | pACH | 0.8750 | 0.0501 | |
| | pNormal | 0.1250 | 0.9499 | |
| | | DCIS | Normal | 3. DCIS classifier |
| | pDCIS | 0.7048 | 0.1869 | |
| | pNormal | 0.2952 | 0.8131 | |
| | | IDC | Normal | 4. IDC classifier |
| | pIDC | 0.7056 | 0.2686 | |
| | pNormal | 0.2944 | 0.7314 | |

*Cancer is any sample with either DCIS or IDC component.

Predicted status for each sample (e.g. pCancer, pADH, pNormal, etc) was compared with its true status (Cancer, ADH, Normal, etc). Intersection of predicted and true status for each type of cancer shows the sensitivity (e.g. 72.39% of Cancer samples are correctly identified, so the sensitivity of cancer classifier is 72.39%), while intersection of predicted and true status of Normals indicates the specificity of the classifier (e.g. 74.74% of Normal samples are correctly identified by the cancer classifier, so its specificity is 74.74%).

5. Classifier Genes

Nine promoters were consistently predictive for cancer classification in all rounds of cross-validation, while 19 were important for ADH classification as indicated in Table 7.

TABLE 7
Genes used for classifier of each sample group

| | Normal | ADH | DCIS | IDC | Cancer* |
|---|---|---|---|---|---|
| | % U | % U (Fisher's Exact Test p-value) | | | |
| EP300 | .167 | .675 | .577 | .474 | .516 |
| | | (<0.001) | (.002) | (0.010) | (0.001) |
| MGMT | .379 | .925 | .852 | .744 | .788 |
| | | (<0.001) | (<0.001) | (0.003) | (<0.001) |
| TP73 | .103 | .750 | .520 | | .410 |
| | | (<0.001) | (0.001) | | (0.003) |
| PGR (distal pr) | .346 | .842 | | .657 | .639 |
| | | (<0.001) | | (0.021) | (0.018) |
| THBS1 | .233 | .750 | | .526 | .515 |
| | | (<0.001) | | (0.024) | (0.014) |
| PYCARD | .200 | .889 | .545 | .706 | .643 |
| (TMS1) | | (<0.001) | (0.018) | (<0.001) | (<0.001) |
| PRKCDBP | .269 | .826 | .647 | | |
| (SRBC) | | (<0.001) | (0.026) | | |
| FABP3 | .333 | | | .724 | .660 |
| (MDGI) | | | | (0.009) | (0.018) |
| MSH2 | .385 | | .875 | | .750 |
| | | | (<0.001) | | (0.003) |
| HIC1 | .100 | | .444 | .395 | .415 |
| | | | (0.006) | (0.011) | (0.002) |
| BRCA1 | .032 | .650 | | | |
| | | (<0.001) | | | |
| TES | .000 | .600 | | | |
| | | (<0.001) | | | |
| NR3C1 (GR) | .032 | .550 | | | |
| | | (<0.001) | | | |
| ICAM1 | .214 | .781 | | | |
| | | (<0.001) | | | |
| DAPK1 | .161 | .600 | | | |
| | | (<0.001) | | | |
| TNFSF11 | .194 | .641 | | | |
| (RANKL) | | (<0.001) | | | |
| DNAJC15 | .346 | .800 | | | |
| (MCJ) | | (<0.001) | | | |
| CDH1 | .308 | .760 | | | |
| | | (.002) | | | |
| CASP8 | .269 | .641 | | | |
| | | (.005) | | | |
| RPL15 | .231 | .550 | | | |
| | | (.012) | | | |
| PGK1 | .179 | .475 | | | |
| | | (.019) | | | |

*Cancer is any sample with either DCIS or IDC component.

The fraction of U calls for each tissue type is shown with p-values from Fisher's Exact Test for differential methylation on 2×2 tables for all pairwise comparisons. These values are reported only as summary statistics. In the cross-validation scheme, gene selection was performed separately for each training set (see text). Blank cells indicate that the gene was not consistently selected in the classifier for the corresponding comparison.

In all cases unmethylated genes were informative; this was consistent with the design of the assay in which a “methylated” signal would be found even when only a fraction of specific templates was methylated (Melnikov A A, et al., Nucleic Acids Res, 33:e93 (2005)). In this respect, the M³-assay performed very similar to the original MSRE-PCR assay (see FIG. 8). In a heterogeneous specimen, a methylated sequence could originate from tumor cells or any other part of the sample; would nonetheless be amplified, and the whole fragment would be scored as methylated. Only unmethylated fragments could be unequivocally assigned to tumor cells and their unmethylated status in other parts of the sample would not change the result of the M³-assay.

### D. Discussion

1. Technical Approach

Abnormal DNA methylation in neoplastic cells can be a valuable biomarker for cancer detection (Herman J G., Chest, 125:119 S-22S (2004); Brena R M, et al., J Mol Med, 84:365-77 (2006)). Unfortunately there is only a limited probability of methylation for each gene (Herman J G, et al., Cancer Res, 55:4525-30 (1995)), so only a combined measurement of multiple methylation biomarkers may provide useful data. The M³-assay is developed to generate such composite biomarkers.

Use of bisulfite degrades the target DNA (up to 95%) (Grunau C, et al., Nucleic Acids Res, 29:E65-5 (2001)), and hence may reduce amplifiable DNA (Munson K, et al., Nucleic Acids Res, 35:2893-903 (2007)). Biased amplification of remaining DNA (sequence-, strand-, and level of methylation-dependent bias) has been reported (Warnecke P M, et al., Nucleic Acids Res, 25:4422-6 (1997)). While these problems may not be significant for homogeneous or ample specimens, they can be critical for heterogeneous clinical specimens and may produce inaccurate results, especially if DNA degradation is specific to certain sequences. In addition, degradation of the major part of a limited clinical sample may prevent its comprehensive analysis that will be also reflected in reduced analytical sensitivity. With this in mind, we have compared bisulfite-based techniques (methylation-specific PCR and bisulfite sequencing) to MSRE-PCR using homogeneous specimens from cultured cells where these problems are less likely to produce biased results (Melnikov A A, et al., Nucleic Acids Res, 33:e93 (2005)). The inherent flaws in the bisulfite technique suggest that an alternative procedure for detection of methylated DNA in clinical samples is needed.

The M³-assay is similar to MSRE-PCR (Melnikov A A, et al., Nucleic Acids Res, 33:e93 (2005)), but relies on microarray-based rather than gel-based signal detection. As in many other DNA methylation techniques, the M³-assay evaluates methylation in a selected number of sites in each gene that may or may not correlate with sites critical for gene expression; this feature makes direct comparison of methylation and expression tenuous (Melnikov A A, et al., Nucleic Acids Res, 33:e93 (2005)). The M³-assay is designed to efficiently detect methylated DNA fragments that can serve as templates for PCR in a heterogeneous sample. In the heterogeneous sample any component can provide such a fragment, making it impossible to explicitly assign methylation to a specific part of the sample, e.g. to neoplastic cells. The absence of PCR product, on the other hand, indicates that no tissue within the sample contains methylated fragments, so the absence of methylation in neoplastic tissue can be unequivocally established. This feature of the M³-assay makes the detection of umethylated genes informative for specimen classification, while detection of methylated genes is uninformative.

Assignment of “methylated” (M) and “unmethylated” (U) calls in the M³-assay depends on the ratio of fluorescence produced by undigested and digested DNA, which in theory can only assume two values: 1/1=1, if the fragment is methylated and digestion has no effect, or 1/0=infinity, if the fragment is unmethylated and no signal from digested DNA is detected. This type of ideal distribution is rarely seen even in cell lines (Melnikov A A, et al., Nucleic Acids Res, 33:e93 (2005)).

Quantitative measurement of signals expressed as Cy5/Cy3 ratio can produce significant discrepancies due to variability of experimental conditions and sampling differences. To manage experimental variability (e.g. the dye bias), SMC is used to define a threshold for methylation (a “self-self” hybridization (Yang Y H, et al., Nucleic Acids Res, 30:e15 (2002))). This approach reduces numerical microarray data to a binary readout (Table 5), simplifies downstream analysis, and reduces the influence of sampling errors. As with the MSRE-PCR, the M³-assay efficiently detects methylated genes. For example, a sample containing equal amounts of methylated and unmethylated fragments (50% unmethylated) produces a “methylated” readout (FIG. 8). Further increase of unmethylated fragment's share drives the Cy5/Cy3 ratio above the SMC level, so these fragments are scored as “unmethylated”. Interestingly, the increase in the Cy5/Cy3 ratio is different for analyzed genes suggesting certain influence of nucleotide composition on dye incorporation; for PAX5 even 10% of methylated fragments keep the Cy5/Cy3 ratio rather low (FIG. 8).

Importantly, the M³-assay is not intended for quantitative assessment of methylation: it is designed for analysis of heterogeneous clinical samples where quantitative differences in methylation can depend on many reasons, including variations in tumor/stroma ratio and presence or absence of inflammation. These variations can be reduced by careful selection of samples, but at the cost of their subjective evaluation.

Another feature of the M³-assay is the internal control for each spot provided by undigested DNA. This control is essential when damaged DNA (e.g. DNA from FFPE samples) is used to ensure that a specific fragment is present. Data processing ignores all spots where hybridization signals for control (undigested) DNA are not detected.

Due to technical challenges of microarray-based techniques, the M³-assay is not intended for immediate clinical use; rather, the M³-assay provides the screening tool for selection of informative genes for a specific disease. Once such genes are identified, other, less demanding techniques can be applied to design the final clinical test.

2. Classifier Genes

The Classifier for Cancer is a combination of DCIS and IDC classifiers (Table 7). For example, TP73 and MSH2 are components of the DCIS but not of the IDC classifier, indicating differences important only to ductal carcinoma in situ. Conversely, PGR, THBS1 and FABP3 are not informative for DCIS classification, but contribute to IDC classification, suggesting that disparities in their methylation status are significant only in invasive cancer.

Most of the promoters that define the Cancer classifier (6/9) are also components of the ADH classifier, a result consistent with previously reported data that cancer-defining methylation changes appear very early in the process (Umbricht C B, et al., Oncogene, 20:3348-53 (2001)) and extending these findings to unmethylated genes. Presence of PRKCDBP within the ADH and DCIS classifiers may indicate methylation changes that are informative during early stages of breast cancer, but not during IDC. U calls for each gene and p-values from Fisher's Exact Test for all pairwise comparisons are shown (Table 7). Blank cells indicate that the gene was not selected for the biomarker.

It is important that a useful biomarker for cancer contains unmethylated rather than methylated genes, because in a heterogeneous tissue, a methylated fragment may be amplified from any part of the sample, so the methylation signal is not necessarily produced by the tumor. Absence of methylation, however, explicitly indicates that the fragment is unmethylated everywhere in the sample, including tumor cells, so the difference in unmethylated genes between healthy tissue and cancer specimen can be used to identify tumors. It is expected that genes that are unmethylated in tumor, but methylated in healthy tissue can be related to tumor growth, de-differentiation, and invasiveness. Indeed, at least some of the genes found in our study meet these criteria (e.g. EP300 (Iyer N G, et al., Proc Natl Acad Sci USA, 101:7386-91 (2004)), TP73, (Beitzinger M, et al., Oncogene, 25:813-26 (2006)), THBS1 (Albo D, et al., J Surg Res, 108:51-60 (2002)), FABP3 (Hashimoto T, et al., Pathobiology, 71:267-73 (2004)).

The larger number of informative promoters identified for the ADH classifier (Table 7) is reflected in a higher accuracy of the ADH classifier (Table 6), suggesting a systematic difference. The most consistent difference is the source of specimens in that all samples of ADH are from core biopsies, whereas other specimens are from gross sections of surgically removed tissues. These gross sections have not been enriched for tumor cells and contain variable amounts of stroma and tumor cells. Compared to gross sections, core biopsies of ADH are by far the most homogeneous.

The similarities in sets of informative genes found for the different stages of breast cancer indicates that no substantial difference can be detected and that differentiation of these stages is currently impossible. These observations raise two distinct possibilities, either that the current set of genes is insufficient to define specific biomarkers for each stage, or that progression of breast cancer from ADH to IDC does not involve molecular differences, at least at the level of DNA methylation. While there is no data to test either hypothesis, we believe that inclusion of additional genes will create a larger analytical space and will provide new biomarkers specific for each stage of breast cancer.

Results of this study may be affected by the age difference in the control and other groups (Table 4), because DNA methylation increases with age (Li L C, et al., Biochem Biophys Res Commun, 321:455-61 (2004)). However, informative genes are chosen for their reduced methylation in abnormal samples, so it is unlikely that age-dependent increase of methylation has significantly influenced the results.

While abnormal promoter methylation is an established feature of breast cancer cells (Widschwendter M, Jones P A., Oncogene, 21:5462-82 (2002)), a diagnostic test based on DNA methylation has yet to be developed. One of the problems is the variability of methylation for each individual fragment. This variability indicates that analysis of a single gene may not provide sufficient accuracy for cancer detection. In the last two years several groups reported multi-gene DNA methylation profiles for detection and classification of breast cancer (Shinozaki M, et al., Clin Cancer Res, 11:2156-62 (2005); Lewis C M, et al., Clin Cancer Res, 11:166-72 (2005); Fiegl H, et al., Cancer Res, 66:29-33 (2006); Li S, et al., Cancer Lett, 237: 272-80 (2006); Fackler M J, et al., Clin Cancer Res, 12:3306-10 (2006)), so the need for multi-gene profiles is widely recognized. The M³-assay is designed to quickly generate such profiles facilitating selection of informative genes that can become targets for a clinical test.

Importantly, the M³-assay produces an integral methylation profile, where the signal from tumor cells is merged with signal from other tissues. As a result the M (methylated) call can be produced by any or all parts of the sample, so the informative value of the M calls is much lower than that of the U (unmethylated), which indicates that the fragment is unmethylated in all parts of the sample. Low informative value of the M calls explains why the composite biomarker contains only the U calls. This feature complicates direct comparison with data from other studies, where hypermethylation of a specific promoter is informative. Results of Fackler et al. (Fackler M J, et al., Clin Cancer Res, 12:3306-10 (2006)) demonstrate this difference: all hypermethylated (and thus informative) promoters of their study tested in our project, are scored as methylated (and thus uninformative) by the M³-assay.

This study shows that complex and heterogeneous samples can be classified if methylation in multiple sites within the same specimen is evaluated. The current version of the assay is still insufficiently accurate and too complex for clinical application; however, it provides the platform for selection of informative genes that can produce a composite biomarker. Furthermore, tissue analysis has only limited clinical utility, and serves only as a proof-of-principle that a combined analysis of multiple informative genes in heterogeneous samples is feasible, and may lead to development of an accurate composite biomarker. It is possible that using the same assay with cell-free circulating DNA may provide a useful approach for cancer detection.

### E. Conclusion

Abnormal DNA methylation is well established for cancer cells, but a methylation-based diagnostic test is yet to be developed. One of the problems is insufficient accuracy of cancer detection in heterogeneous clinical specimens when only a single gene is analyzed. A new technique was developed to produce a multi-gene methylation signature in each sample, and its potential for selection of informative genes was tested using DNA from formalin-fixed paraffin embedded breast cancer tissues. Fifty six promoters were analyzed in each of 138 clinical specimens by a microarray-based modification of the previously developed technique. Specific methylation signatures were identified for atypical ductal hyperplasia, ductal carcinoma in situ, and invasive ductal carcinoma. Informative promoters selected by Fisher's Exact Test were used for composite biomarker design using naïve Bayes algorithm. All informative promoters were unmethylated in disease as compared to normal tissue. Cross-validation showed 72.4% sensitivity and 74.7% specificity for detection of ductal carcinoma in situ and invasive ductal carcinoma, and 87.5% sensitivity and 95% specificity for detection of atypical ductal hyperplasia. These results indicate that informative cancer-specific methylation signatures can be detected in heterogeneous tissue specimens, suggesting that a diagnostic assay can then be developed.

### Example III

### A. Introduction

Despite its relatively low prevalence (40 cases per 100,000 women per year (Jemal A, et al., CA Cancer J Clin, 55:10-30 (2005)) ovarian cancer is the most frequent cause of death from gynecological malignancies. The vast majority of ovarian tumors occur in postmenopausal women; at early stages they are mostly asymptomatic or present with vague and non-specific symptoms. As a result, early ovarian cancer is difficult to diagnose, and almost 90% of patients are diagnosed at an advanced stage with metastases in the pelvis or abdomen. For these patients surgical and chemotherapeutic management have limited impact with 5-year survival rates being less than 30%. In contrast, patients diagnosed with stage I ovarian cancer have a 5-year survival rate in excess of 90%, strongly suggesting that screening for early detection of ovarian cancer may reduce cancer-related mortality.

It has been suggested that a screening test for ovarian cancer should have a positive predictive value of 10% or more; then 10 women would undergo exploratory surgery to diagnose one cancer (Bast R C, Jr., et al., Recent Results Cancer Res, 174:91-100 (2007). Considering the low prevalence of ovarian cancer in the general population the screening test would need a sensitivity of at least 75% and a specificity of at least 99.6% to achieve this positive predictive value. The screening test should also be simple, inexpensive, and produce only minimal discomfort for women.

Such a test has yet to emerge. A blood-based test developed by R. Bast and coworkers, (Bast R C, Jr., et al., J Clin Invest, 68:1331-7 (1981), which measures cancer antigen 125 (CA 125), is currently the most widely used procedure for ovarian cancer detection and monitoring (Yurkovetsky Z R, et al., Future Oncol, 2:733-41 (2006; Munkarah A, et al., Curr Opin Obstet Gynecol, 19:22-6 (2007)). The specificity of CA 125 for early-stage disease is high (96-100%), but the sensitivity is relatively unimpressive ranging between 40% (Jacobs 1, et al., Bmj 306:1030-4 (1993); Skates S J, et al., J Clin Oncol 22:4059-66 (2004)), and 60% (Bast R C, Jr., J Clin Oncol, 21:200s-205s (2003)). Low sensitivity indicates that CA 125 test alone is insufficient for diagnosis and has to be combined with other types of analysis. A two-line screening procedure can be performed: first, the CA 125 test identifies candidates with higher than normal CAl25, who then undergo the second line procedure, transvaginal ultrasonography (TVUS) (Bast R C, Jr., et al., Recent Results Cancer Res, 174:91-100 (2007); Bast R C, Jr., et al., Int J Gynecol Cancer, 15 Suppl 3:274-81 (2005)).

Unfortunately, a combination of CA125 and TVUS still has only a limited sensitivity because of low sensitivity of the initial CA 125 test (Menon U, et al., Bjog, 107:165-9 (2000), even when women from a high-risk group are screened the test still does not provide considerable advantages (van Nagell J R, Jr., et al., Gynecol Oncol, 77:350-6 (2000); Fishman D A, et al., Am J Obstet Gynecol, 192:1214-22 (2005); Stirling D, et al., J Clin Oncol, 23:5588-96 (2005); Fields M M, Chevlen E., Clin J Oncol Nurs, 10:77-81 (2006)). In addition, the test does not detect tumors at a sufficiently early stage to influence outcomes (Stirling D, et al., J Clin Oncol, 23:5588-96 (2005); Olivier R1, et al., Gynecol Oncol, 100:20-6 (2006)). As a result, low sensitivity and a high rate of false-negative results of the CA125 test reduce access to TVUS for women who might have benefited from this procedure; on the other hand, low sensitivity of TVUS for early cancer suggests that even if it was done, the effect on prognosis would have been negligible (Stirling D, et al., J Clin Oncol, 23:5588-96 (2005); Olivier R I, et al., Gynecol Oncol, 100:20-6 (2006)).

To improve detection rates different combinations of CA 125 with other antigens have been suggested (Skates S J, et al., J Clin Oncol 22:4059-66 (2004); Bast R C, Jr., et al., Int J Gynecol Cancer, 15 Suppl 3:274-81 (2005); Rosen D G, et al., Gynecol Oncol, 99:267-77 (2005); Scholler N, et al., Clin Cancer Res, 12:2117-24 (2006); Moore L E, et al., Cancer Epidemiol Biomarkers Prey, 15:1641-6 (2006); Diefenbach C S, et al., Gynecol Oncol, 104:435-42 (2007)) indicating the trend towards evaluation of multiple biomarkers for improved detection. The current paradigm involves combinations of serum markers as the first line of screening followed by TVUS for confirmation (Bast R C, Jr., et al., Recent Results Cancer Res, 174:91-100 (2007); Munkarah A, et al., Curr Opin Obstet Gynecol, 19:22-6 (2007)); the major focus remains on proteins and only a few attempts are made to use other markers, including DNA. Meanwhile DNA is a relatively stable molecule, which can be readily amplified in polymerase chain reaction to provide high analytical sensitivity; it can be recovered from blood of ovarian cancer patients (e.g. (Chang H W, et al., J Natl Cancer Inst, 94:1697-703 (2002); Kamat A A, et al., Cancer Biol Ther, 5:1369-74 (2006)), and can be used as a biomarker directly (Kamat A A, et al., Acad Sci, 1075:230-4 (2006)) or as a substrate to test for the presence of mutations (e.g. in p53 (Okuda T, et al., Gynecol Oncol, 88:318-25 (2003)). It can also be used to test for abnormal DNA methylation, which has been found in ovarian tumors (Dhillon V S, et al., Br J Cancer, 90:874-81 (2004); Kassim S, et al., IUBMB Life, 56:417-26 (2004); Kaneuchi M, et al., Biochem Biophys Res Commun, 316:1156-62 (2004); Yang H J, et al., BMC Cancer, 6:212 (2006); Wiley A, et al., Cancer, 107:299-308 (2006)); this option is explored in our work.

Considering that methylation of a single gene is unlikely to provide diagnostic accuracy at the level required for screening of the asymptomatic population, we hypothesized that a combination of several informative genes (a composite biomarker) would increase accuracy of detection. This task requires development of methylation profiles with multiple genes in order to identify the most informative genes. In this proof-of-principle project we sought to confirm that this approach can eventually produce a sufficiently accurate composite biomarker. We tested the methylation status of 56 promoters in DNA extracted from ovarian tumors and from unaffected ovaries. To confirm that a similar approach can be used for blood-based detection, we analyzed methylation profiles of cell-free plasma DNA from cancer patients and healthy controls.

### B. Materials and Methods

1. Clinical Specimens

The project was approved by the Institutional Review Board at Northwestern University. Tissues: formalin-fixed paraffin-embedded (FFPE) tissues were provided by Pathology Core Facility of the Robert H. Lurie Comprehensive Cancer Center, Feinberg School of Medicine, Northwestern University. Serous papillary adenocarcinoma (stage 3 in over 80% of samples) with mostly endometrioid components was selected as the most frequent type of ovarian tumors; tumor description from the Surgical Pathology final report was confirmed by a single pathologist. Control group included ovarian tissues from subjects of the high-risk group defined as women with family history of ovarian cancer, personal history of breast cancer or women with a mutation in BRCA1 gene; in most cases follicular and luteal cysts were present in removed ovaries. Plasma from women with serous papillary adenocarcinoma was provided by the Fox Chase Cancer Center Biosample Repository. Blood specimens were collected from ovarian cancer patients prior to tumor removal or initiation of chemotherapy. Stage of the disease and tumor grade was extracted from the Surgical Pathology final report. Plasma from healthy female volunteers of similar age and race was deposited in the same Repository. A brief description of samples including stage of the disease, grade of the tumor, and age of donors is presented in Table 8.

TABLE 8

| | | |
|---|---|---|
| Age | Stage | Grade |

| | | |
|---|---|---|
| Mean | Range | Range |

Tissue specimens

| | | | | |
|---|---|---|---|---|
| Disease | 59 | 29-80 | 1c-4 | 1-3 |
| Control | 47.4 | 32-61 | NA | NA |

Plasma specimens

| | | | | |
|---|---|---|---|---|
| Disease | 65 | 50-80 | 3a-4 | 1-3C |
| Control | 65 | 50-81 | NA | NA |

2. DNA isolation

One 10 micron section from a paraffin block was used for DNA isolation. After xylene deparaffination and ethanol precipitation, the tissue pellet was processed using a DNeasy Tissue kit (Qiagen, Valencia, Calif.). Purified DNA was dissolved in 10 mM Tris pH7.8, 0.5 mM EDTA. DNA from plasma (0.2 ml) was purified using DNAzol reagent (Molecular Research Center, Cincinnati, Ohio).

3. Microarray Mediated Methylation Assay: Overall Approach

In the microarray mediated methylation assay (M³-assay), one portion of each genomic DNA sample was digested with a methylation-sensitive restriction enzyme while another portion of the same sample served as an undigested control. Selected regions of the genomic DNA from each of the digested and undigested DNA samples were amplified by PCR using gene-specific primers that flank restriction sites. For the amplified product from the digested portion only fragments with methylated sites were capable to serve as templates, whereas in the undigested (control) portion, all fragments were amplified. Comparison between the two sets of PCR products was done by gel electrophoresis (MSRE-PCR) (Melnikov A A, et al., Nucleic Acids Res, 33:e93 (2005)) or by competitive hybridization with custom-designed microarrays (M³-assay). Fluorescent signals of hybridized fragments in the M³-assay were separately scored, and the ratio between the signals from control and digested DNAs was calculated. This ratio was used to assign “methylated” or “unmethylated” calls to the targeted regions. The data were statistically assessed to select groups of informative fragments, which were then analyzed together as a composite biomarker. Details of the method are presented below.

4. DNA Digestion

Hin6I (Fermentas, Hanover, Md.) was used to digest one half of each purified genomic DNA sample as described (Melnikov A A, et al., Nucleic Acids Res, 33:e93 (2005)). The second half of each DNA sample was incubated in the digestion buffer but without the enzyme and served as the control.

5. PCR Amplification

Nested PCR was performed as described (Melnikov A A, et al., Nucleic Acids Res, 33:e93 (2005)). KlenTaq1 (Barnes W M., Proc Natl Acad Sci USA, 91:2216-20 (1994)) (DNA Polymerase Technology, St. Louis, Mo.) was used at 8 U per 30 μl reaction. Betain and dNTPs (Sigma, St. Louis, Mo.) were added to the PCR buffer to 1.5M and 0.25 mM, respectively. The PCR reaction was assembled on ice, the tubes were placed into a thermocycler (ABI 9600, Applied Biosystems, Foster City, Calif.), incubated at 95° C. for 5 min, and KlenTaq1 was added. After 25 cycles (95° C.; 45 sec—62° C.; 1 min—72° C.; 1 min) the products were precipitated, dissolved in TE, and 1.5 ng was used for the second PCR, assembled with aminoallyl-dUTP (Biotium, Hayward, Calif.) and dTTP (3:1), and performed as the first. PCR products were precipitated, and dissolved for labeling in 20 μl of 100 mM NaHCO₃ buffer (pH 9.0).

6. DNA Labeling

Five microliters of Cy3 or Cy5 (Monoreactive Dye Pack, Amersham, Piscataway, N.J.) in DMSO were dried in a vacuum. PCR products in 100 mM NaHCO₃ buffer (pH 9.0) were added, and the reaction was allowed to proceed for 2 hrs at room temperature. Unreacted dyes were quenched by 10 μl of 4M hydroxylamine, and the labeled products were precipitated. The PCR products from undigested (control) DNA were labeled with Cy5, while Cy3 was used to label the PCR products from Hin6I-digested DNA.

7. Methylation Assay (MethDet-Assay)

DNA methylation analysis was done as described (Melnikov A A, et al., Nucleic Acids Res, 33:e93 (2005)) except microarray-based detection was used. PCR products from undigested DNA were labeled with Cy5, while those from digested DNA—with Cy3. Labeled products were mixed and hybridized to the custom-designed microarray that contained probes for 56 promoter fragments and five controls (Table 9).

TABLE 9

| Official Symbol | Official Full Name | Alias | Other Designations | GenBank ID |
|---|---|---|---|---|
| ABCB1 | ATP-binding cassette, sub-family B, | MDR1 | multidrug resistance, gene 1 | X58723 |
| | member 1 | | | |
| ACTB | actin beta | | | Y00474 |
| ACTB* | actin beta (cDNA) | | | X63432 |
| APAF1 | apoptotic peptidase activating factor | | | AC013283 |
| Arabidopsis* | | | | |
| BRCA1 | Breast cancer 1, early onset | | breast and ovarian cancer | U37574 |
| | | | susceptibility protein 1 | |
| CALCA | calcitonin/calcitonin-related polypeptide, alpha | | | X15943 |
| CASP8 | caspase 8, apoptosis-related cysteine peptidase | | | AB038980 |
| CCND2 | cyclin D2 | | | U47284 |
| CDH1 | Cadherin 1 | | E-cadherin | L34545 |
| CDKN1A | cyclin-dependent kinase inhibitor 1A | p21waf1, | | AF497972 |
| | | p21cip1 | | |
| CDKN1B | cyclin-dependent kinase inhibitor 1B | p27kip1 | | AB005590 |
| CDKN1C | cyclin-dependent kinase inhibitor 1C | p57kip2 | | D64137 |
| CDKN2A | cyclin-dependent kinase inhibitor 2A | p16INK4A | | NT_037734 |
| CDKN2B | cyclin-dependent kinase inhibitor 2B | p15INK4B | | NT_037734 |
| DAPK1 | death-associated protein kinase 1 | | death-associated protein | AL161787 |
| | | | kinase | |
| DNAJC15 | DnaJ (Hsp40) homolog, subfamily C, | MCJ | methylation-controlled J | NT_033922 |
| | member 15 | | protein | |
| EDNRB | endothelin receptor type B | | | AF114163 |
| EP300 | E1A binding protein p300 | | | AL080243 |
| ESR1 promoter A | estrogen receptor 1 | ER alpha | estrogen receptor alpha | AL356311 |
| ESR1 promoter B | estrogen receptor 1 | ER alpha | estrogen receptor alpha | AL356311 |
| FABP3 | fatty acid binding protein 3 | MDGI | mammary-derived growth | U17081 |
| | | | inhibitor | |
| FAS | Fas (TNF receptor superfamily, member 6) | | | X87625; |
| | | | | D31968 |
| FHIT | fragile histidine triad gene | | | AF399855 |
| GAPDH* | glyceraldehyde-3-phosphate dehydrogenase (cDNA) | | | X01677 |
| GPC3 | glypican 3 | | | AF003529 |
| GSTP | glutathione S-transferase pi | | | M37065 |
| HIC1 | hypermethylated in cancer 1 | | | L41919 |
| HLTF | helicase-like transcription factor | | | Z46606 |
| ICAM1 | intercellular adhesion molecule 1 | CD54 | | M65001 |
| MCTS1 | malignant T cell amplified sequence 1 | MCT1 | | AC011890 |
| MGMT | O-6-methylguanine-DNA methyltransferase | | | X61657 |
| MLH1 | mutL homolog 1 | | | AC011816 |
| MSH2 | mutS homolog 2 | | | AB006445 |
| MUC2 | mucin 2, intestinal/tracheal | | | U67167 |
| MYOD1 | myogenic differentiation 1 | MYF-3 | myogenic factor 3 | AC124056 |
| NR3C1 | nuclear receptor subfamily 3, group | GR | glucocorticoid receptor | M69074 |
| | C, member 1 | | | |
| PAX5 | paired box gene 5 | | | AF268279 |
| PGK1 | phosphoglycerate kinase 1 | | | M34017 |
| PGR dist | progesterone receptor | PR | | X51730 |
| PGR prox | progesterone receptor | PR | | X51730 |
| PLAU | plasminogen activator, urokinase | uPA | urokinase plasminogen | X02419 |
| | | | activator | |
| PRDM2 | PR domain containing 2, with ZNF | RIZ1 | retinoblastoma protein- | AF472587 |
| | domain | | interacting zinc finger protein | |
| PRKCDBP | protein kinase C, delta binding protein | SRBC | serum deprivation response | AF408198 |
| | | | factor (sdr)-related gene | |
| | | | product that binds to c-kinase | |
| PYCARD | PYD and CARD domain containing | TMS1 | target of methylation-induced | AF184072 |
| | | | silencing-1 | |
| RARB | retinoic acid receptor, beta | RAR beta 2 | retinoic acid receptor, beta 2 | X56849 |
| RASSF1 | Ras association (RaIGDS/AF-6) | RASSF1A | | AC002481 |
| | domain family 1 | | | |
| RB1 | retinoblastoma 1 | | | AL392048 |
| RPL15 | ribosomal protein L15 | | | AB061823 |
| S100A2 | S100 calcium binding protein A2 | | | AL162258 |
| SCGB3A1 | secretoglobin, family 3A, member 1 | HIN1 | high in normal-1 | NT_006519 |
| SFN | stratifin | 14-3-3 s | 14-3-3 sigma | AF029081 |
| SLC19A1 | solute carrier family 19 (folate | RFC1 | reduced folate carrier | U92868 |
| | transporter), member 1 | | | |
| SOCS1 | suppressor of cytokine signaling 1 | | | Z46940 |
| SYK | spleen tyrosine kinase | | | AC021581 |
| TES | testis derived transcript | | | AJ250865 |
| THBS1 | thrombospondin 1 | | | J04835 |
| TNFSF11 | tumor necrosis factor (ligand) | TRANCE, | osteoprotegerin ligand | AF333234 |
| | superfamily, member 11 | RANKL, | | |
| | | OPGL | | |
| TP73 | tumor protein p73 | p73 | | AF235000 |
| TUBA3* | Tubulin alpha 3 (cDNA) | | | K00558 |
| VHL | von Hippel-Lindau tumor suppressor | | | AF010238 |

Sequences marked with (*) were used as negative controls.

Three identical sub-arrays were spotted on each slide, so hybridization signal was confirmed in triplicate. Cy5 and Cy3 signals were filtered to exclude unreliable data (signal intensity comparable to or less than background), and the background was subtracted before ratios of Cy5/Cy3 were calculated for each spot.

To avoid labeling variability due to the sequence differences we determined individual Cy5/Cy3 ratios for each completely methylated fragment using “self-self” assay (Yang I V, et al., Genome Biol, 3:research0062 (2002)). PCR products from control (undigested) DNA were divided into two equal aliquots, labeled with either Cy3 or Cy5, mixed, and used for hybridization. This design assured equal representation of Cy3- and Cy5-labeled fragments as if DNA was methylated, so the ratio of intensities defined a methylation threshold for each promoter (standard methylation call, SMC). SMCs were used to assign calls to each gene; an example of data is shown (Table 10). If no call can be assigned, the gene was scored as NA (none assigned).

TABLE 10

| | Signal from | | Ratio | Methylation |
|---|---|---|---|---|
| Gene | Cy5 | Cy3 | Cy5/Cy3 | calls |
| DNAJC15 | 64504 | 36053 | 1.8 | M |
| MCTS1 | 64561 | 33619 | 1.9 | M |
| ICAM1 | 64504 | 32923 | 2 | M |
| MGMT | 64509 | 17836 | 3.6 | M |
| TNFSF11 | 15402 | 1389 | 11.1 | UM |
| CDH1 | 6044 | 508 | 11.9 | UM |
| BRCA1 | 51208 | 3997 | 12.8 | UM |
| EP300 | 64551 | 4781 | 13.5 | UM |
| PAX5 | 64423 | 2336 | 27.6 | UM |

8. Hybridization and Signal Detection

Custom-designed arrays (MWG Bioinformatics, High Point, N.C.) containing 60-mer probes for each amplified product were printed as a 8×8 grid on aminosilane-modified glass by Microarrays, Inc (Nashville, Tenn.). Each array contained three identical sub-arrays, so the signal was confirmed in triplicate. Out of 64 spots in each sub-array 61 contained probes and three were empty. Four control probes in each sub-array were designed to control non-specific binding; three of them were derived from cDNA and one—from DNA of Arabidopsis thaliana. Out of remaining 57 (61−4=57) promoter-specific probes in each sub-array one did not pass quality control, leaving 56 promoter-specific probes to be tested. Slides were pre-hybridized for 1 hr at 42° C. in 5×SSC, 0.1% SDS, 1% BSA, rinsed with deionized water and dried. Labeled DNA was dissolved in the hybridization buffer (100 μl; Ocimum Biosolutions, Indianapolis, Ind.), denatured (2 min; 95° C.), and quenched on ice. Microarray GeneFrames (AbGene, Rochester, N.Y.) were used to create space between the slide and the coverslip. Denatured DNA was added, the coverslip was sealed, and the slides were incubated 18 hr at 42° C. The GeneFrame and the coverslip were removed, and the slides were washed at 42° C. for 5 mm in 1×SSC, 0.1% SDS; and twice for 5 min in 0.1×SSC, 0.1% SDS. Slides were scanned using ScanArray XL4000 (Perkin Elmer, Boston, Mass.; sensitivity ≦0.1 molecule per μm²) with ScanArray™ software. Intensity of each fluorophor was measured for each spot, and the background values were subtracted. Ratios of Cy5/Cy3 fluorescence were calculated to compare the yields of PCR products from control and Hin6I-digested DNA.

9. Statistical Analysis

Methylation calls were made independently for each spot, and final gene-specific calls were made according to the majority call from the triplicate spots for that gene. If there was no majority, the final call was NA. As with expression microarray analysis (Scholtens D, von Heydebreck, A., H. W. Gentleman R, Irizarry R, Dudoit S, (2005), Springer), non-specific filtering removed uninformative spots (detectable calls in less than ⅔ of the samples or less than 10% differential methylation across the entire sample set). Informative genes with p<0.10 were selected by Fisher's Exact Test for differential methylation in gene-specific analyses comparing methylation status for cancer and normal samples. The moderate p-value of 0.10 was chosen to include informative genes with occasionally inflated p-values. The apparent independence of methylation sites (Model F, et al., Bioinformatics, 17 Suppl 1:S157-64 (2001)) suggested selection of the naïve Bayes classifier (Domingos P, Michael J. Pazzani, Machine Learning, 29:103-130 (1997)). Naïve Bayes classifiers were constructed using the e1071 R (R Development Core Team, 2005) package (Gentleman R C, et al., Genome Biol, 5:R80 (2004)), using an uninformative prior with probabilities of 0.5 for normal or cancer classification. The predictive ability of the naïve Bayes classifier was estimated using 25 rounds of five-fold cross-validation. For each round of cross-validation, the data were partitioned into five sets with an equal distribution of diseased and control specimens. Each set then served as a test set based on training of the naïve Bayes classifier with the other four sets. Sensitivity and specificity were estimated and averaged over all five runs and over 25 random partitionings of the data into five groups. Gene selection and classifier parameter estimation were performed anew with each round of cross-validation.

### C. Results

1. Clinical Specimens

Age of subjects and tumor descriptions are presented in Table 8 for tissues and plasma samples. Serous papillary adenocarcinoma is the most frequent form of ovarian cancer (Jemal A, et al., CA Cancer J Clin, 55:10-30 (2005)) so its successful detection would have the strongest impact. Most (26 of 30 or 86.7%) of ovarian cancer cases (n=30) had advanced disease (stage 3b and higher), and only 4 cases had lower stages. Most of the tumors were either moderately or poorly differentiated (90% grade 2 or higher) and only 3 tumors were either grade 1 or borderline. Histology of the tumors was predominantly serous papillary adenocarcinoma (70%) with additional endometrioid components present in 30% of the cases. As ovarian tissues from healthy women were not available, control group (n=30) contained tissues from women at high risk for ovarian cancer undergoing preventive bilateral salpingo-oophorectomy. This group included women with family history of ovarian cancer, with personal history of breast cancer, and six women had confirmed mutations of BRCA1 (Kauff N D, Barakat R R., J Clin Oncol, 25: 2921-7 (2007)). No neoplastic changes were detected in specimens from this group, although a possibility of occult neoplasia could not be excluded. Most of the samples (83.3%) contained multiple cysts, including hemorrhagic and paratubal cysts. Five specimens contained benign tumors (cystadenoma, adenofibroma, teratoma), and surface epithelial hyperplasia was noted for two samples. Cancer cases were on average older than controls, with mean age 59 vs. 47.4 (p<0.001 using two sample t-test).

Plasma samples were obtained from a different cohort of healthy women (n=33) and women with serous papillary adenocarcinoma (n=33). These samples were collected prior to surgery and/or chemotherapy. Cases and controls were age-matched (average age 65 in both groups). All cancer cases had disease at stage 3A or higher; of the 22 cases where tumor grade was established only 3 had well-differentiated (grade 1), while all the rest were poorly differentiated (grade 3 and higher).

2. Genes of the Composite Biomarker

Ten genes were found to be consistently predictive for ovarian cancer detection in multiple rounds of cross-validation when tissue samples were used, while five were important for cancer detection using plasma samples (Table 11).

TABLE 11
Unmethylated genes of the composite biomarkers

| | |
|---|---|
| Control | Cancer |

A. Tissue

| | | | |
|---|---|---|---|
| TISSUE | BRCA1 | 20 (66.7%) | 8 (26.7%) |
| | EP300 | 17 (56.7%) | 9 (30%) |
| | NR3C1 (GR) | 19 (63.3%) | 5 (16.7%) |
| | MLH1 | 22 (73.3%) | 7 (23.3%) |
| | DNAJC15 (MCJ) | 21 (70%) | 11 (36.7%) |
| | CDKN1C (p57kip2) | 19 (63.3%) | 3 (10%) |
| | TP73 | 25 (83.3%) | 8 (26.7%) |
| | PGR (prox) | 16 (53.3%) | 1 (3.3%) |
| | THBS1 | 27 (90%) | 12 (40%) |
| | PYCARD (TMS1)* | 20 (76.9%) | 9 (34.6%) |

N = 30 for each group
B. Plasma

| | | | |
|---|---|---|---|
| PLASMA | BRCA1 | 16 (48.5%) | 2 (6.1%) |
| | HIC1 | 16 (48.5%) | 7 (21.2%) |
| | PAX5 | 14 (42.4%) | 7 (21.2%) |
| | PGR (prox) | 18 (54.5%) | 6 (18.2%) |
| | THBS1 | 16 (48.5%) | 3 (9.1%) |

N = 33 for each group
*TMS was detected in 26 samples
Listed are the raw number of times each gene has been scored as unmethylated. The percent of unmethylated scores for each group (Control or Cancer) is presented in parentheses.

In all cases hypomethylation was significant for the classification value, which is consistent with the design of the assay to over-represent methylated fragments (Melnikov A A, et al., Nucleic Acids Res, 33:e93 (2005)). Additionally, only unmethylated promoters in a heterogeneous specimen can be unequivocally assigned to tumor cells; their unmethylated status in other cells will not be reflected in the MethDet-assay. The reverse is not true: methylated promoters will produce a signal regardless of their origin within the heterogeneous specimen, so their informative value is very low.

A combination of genes was used for classification of samples; each of these genes was evaluated for methylation in the set of samples, so that their combined values for the whole set were contributing to the composite biomarker. Individual informative genes exhibited higher level of methylation in cancer samples compared to controls (Table 11), although none of them was exclusively methylated or unmethylated in all samples of any group.

Statistical evaluation of results was done as described in Materials and Methods and sensitivity and specificity of the assay were calculated (Table 12).

TABLE 12
Accuracy of detection
TISSUE SPECIMENS

| | TRUE | |
|---|---|---|
| PREDICTED | Cancer | Normal |
| pCancer | 0.694 | 0.298 |
| pNormal | 0.306 | 0.702 |

PLASMA SPECIMENS

| | TRUE | |
|---|---|---|
| PREDICTED | Cancer | Normal |
| pCancer | 0.851 | 0.389 |
| pNormal | 0.149 | 0.611 |

Sensitivity was determined as the number of positive tests among the cancer cases divided by the total number of cancer cases. Specificity was determined as the number of negative tests among the controls divided by the total number of controls.

### D. Discussion

Current knowledge of ovarian cancer is insufficient for development of mechanistic biomarkers, while carefully designed and tested correlative biomarkers can improve cancer treatment and provide insights into mechanisms of cancer growth. Correlative biomarkers based on abnormal DNA methylation have a significant appeal, because multiple individual markers (differentially methylated CpG sites) are present in each sample and can be analyzed as a group, while the use of PCR ensures that the analytical sensitivity of the technique is extremely high. In addition, abnormally methylated DNA has been consistently detected in bloodstream of patients with different cancers, including ovarian cancer; this provides the opportunity to develop a minimally invasive test that can be used for regular screening of asymptomatic women. The test has to accommodate the inherent heterogeneity of DNA extracted from tumor or blood in order to be clinically applicable. In this project we have explored the feasibility of a sensitive and specific methylation biomarker for ovarian cancer detection based on DNA extracted from ovarian tumors or from patients' blood.

Clinical specimens are heterogeneous by nature, so diagnostic tests have to incorporate sample heterogeneity into their design. In this report we evaluated the possibility of an observer-independent assay for DNA methylation applied to detection of ovarian cancer (serous papillary adenocarcinoma) in clinical samples—tissues and plasma. While the developed test cannot be immediately used for ovarian cancer detection, the results indicate that the approach has obvious merits and that cancer detection by methylation profiling is indeed practical.

The assay includes two stages: detection of methylation by MSRE digestion and detection of the signal for each promoter fragment. Previously validated (Melnikov A A, et al., Nucleic Acids Res, 33:e93 (2005)) procedure has been used for methylation detection. Briefly, analytical sensitivity of the assay is at least 60 pg (for one gene in MSRE-PCR (Bhandare D J, et al., Clin Chim Acta, 367:211-3 (2006)) to 100 pg (for multiple genes in M³-assay, data not shown). During development of the MethDet the efficiency of Hin6I digestion has been controlled by real-time PCR for selected genes (Melnikov A A, et al., Nucleic Acids Res, 33:e93 (2005)); internal control for the M³-assay is provided by detection of unmethylated genes, while preservation of methylation patterns has been observed for both MSRE-PCR and M³-assay in experiments with increased digestion (data not shown). Similar if not identical methylation patterns are detected by the MSRE-PCR and bisulfite-based assays (methylation-sensitive PCR and bisulfite sequencing)(Melnikov A A, et al., Nucleic Acids Res, 33:e93 (2005)); comparison of MSRE-PCR data with published results reveals a remarkable degree of correlation (Melnikov A A, et al., Nucleic Acids Res, 33:e93 (2005)). By design and similar to MSP MethDet evaluates methylation only in a few CpG sites in each promoter, so it would be difficult to expect rigorous correlation between gene expression and MethDet results; although these results correlate well with expression of certain genes (Melnikov A A, et al., Nucleic Acids Res, 33:e93 (2005)), this correlation is likely to be imprecise. For heterogeneous samples this correlation is probably especially tenuous: a positive methylation signal may be generated from a methylated and possibly repressed component while a positive expression signal may be produced from an unmethylated and thus active part of the same specimen. To validate the microarray-based detection platform we have compared results of MSRE-PCR and M³-assay: in eight repeat experiments using genomic DNA from MCF-7, only two genes showed significant differences (2:51=0.39 or 3.9%; data not shown).

It should be noted that control (undigested) DNA is amplified with the same sets of primers side by side with the digested DNA, so controllable parameters (DNA concentration, amplicon length, primer concentration, etc.) are exactly the same. Each specimen contains multiple genes that produce high signal in digested sample and are scored as “methylated”. These genes provide a certain level of assurance that amplification of methylated genes is equally efficient for digested and control samples. At the same time each sample contains several genes that are scored as “unmethylated”, and provide confirmation that Hin6I digestion is efficient.

Initially, we have used the MethDet test (see Materials and Methods) to compare DNA methylation in ovarian tumors and in ovaries without histologically noticeable neoplastic growth. It is important to note that most tissue specimens in the control group have been collected from women of the high-risk group (family or personal history of breast cancer, family history of ovarian cancer, and mutations in BRCA1 gene), so the possibility of an occult neoplasm, which will affect the accuracy of the test, has to be considered.

This part of the project has been designed to establish whether any differences in methylation can be detected by MethDet. Indeed, ten out of 56 genes contribute to the composite biomarker (Table 11) indicating that differential methylation can be detected in heterogeneous samples of ovarian tumors and normal ovaries. Tumors are characterized by increased frequency of methylation in all of the contributing genes. Complete or partial inactivation of several of them is well-established in ovarian cancer: BRCA1 is either mutated (Geisler J P, et al., J Natl Cancer Inst, 94:61-7 (2002)) or its promoter is methylated (Wilcox C B, et al., Cancer Genet Cytogenet, 159:114-22 (2005); Chiang J W, et al., Gynecol Oncol, 101:403-10 (2006)); LOH is frequent in 22q13 locus that contains EP300 (Bryan E J, et al., Int J Cancer, 102:137-41 (2002)); a combination of LOH and methylation is found for DNAJC15 (MCJ)⁴² and MLH1 (Gifford G, et al., Clin Cancer Res, 10:4420-6 (2004); Arzimanoglou, II, et al., Anticancer Res, 22:969-75 (2002)); frequent methylation is observed in promoters of TP73 (Strathdee G, et al., Am J Pathol, 158:1121-7 (2001)) and PYCARD (TMS1) (Terasawa K, et al., Clin Cancer Res, 10:2000-6 (2004)). For other genes (CDKN1C(p57), PGR, and THBS1) there is a good correlation between increased methylation in tumors (this study) and reduced expression in ovarian cancer (Sui L, et al., Anticancer Res, 22:3191-6 (2002); Akahira J, et al., Jpn J Cancer Res, 93:807-15 (2002); Lee P, et al., Gynecol Oncol, 96:671-7 (2005); Kodama J, et al., Anticancer Res, 21:2983-7 (2001)).

The accuracy of cancer detection has been established by stratified cross-validation as described in Materials and Methods. Both sensitivity and specificity have been only fair (Table 12); this can depend on the presence of tissues with occult neoplasia in the control group and/or on the suboptimal selection of genes for MethDet assay. While only moderate accuracy has been achieved for tissue samples, we nonetheless demonstrated that multiplexed analysis of DNA methylation in heterogeneous samples can produce meaningful results and these results can be used for tumor detection.

Analysis of methylation in circulating DNA holds a greater promise for cancer screening, so we have analyzed cell-free circulating DNA from ovarian cancer patients and healthy gender- and age-matched controls. In this case, the sensitivity of plasma-based detection has been considerable (85%), but the specificity has been unacceptably low (Table 12).

Only five genes are required for detection using circulating DNA (Table 11), and three of them (BRCA1, PGR, and THBS1) are parts of the tissue-based composite biomarker panel as well. Among other genes of the biomarker methylation of HIC1 has been identified in ovarian tumors (Strathdee G, et al., Am J Pathol, 158:1121-7 (2001); Rathi A, et al., Clin Cancer Res, 8:3324-31 (2002); Teodoridis J M, et al., Cancer Res, 65:8961-7 (2005); Tam K F, et al., J Cancer Res Clin Oncol, 133:331-41 (2007)), but PAX5 involvement has not been reported previously. Our results correlate well with data from the Cairns' group, who described increased methylation of BRCA1 and RASSF1 in serum of ovarian cancer patients (Ibanez de Caceres I, et al., Cancer Res, 64:6476-81 (2004)); while RASSF1 is among the genes tested, it has not been selected as an informative gene by the naïve Bayes algorithm. The same is true for hypermethylation of MLH1, which has been identified as a predictor of poor survival for ovarian cancer patients after carboplatin/taxol chemotherapy (Gifford G, et al., Clin Cancer Res, 10:4420-6 (2004)).

While it would be premature to apply results of this communication to a clinical trial, high sensitivity of blood-based detection achieved in this proof-of-principle project strongly suggests that the chosen approach can be optimized. One of the obvious directions is improvement of target selection for MethDet: if high sensitivity can be achieved within the existing analytical space of 56 promoters, it is reasonable to expect that a rational choice of targets will improve the accuracy to the level compatible with screening. The relatively high sensitivity of cancer detection in the blood-based assay (85%) suggests that MethDet can be considered as the first-line test in combination with TVUS or other imaging techniques. Finally, samples from the late stages of ovarian cancer have been used in this work. While the most informative targets may be stage-specific, and additional optimization may be required for an early screening test, it appears that a composite biomarker for ovarian cancer based on methylation detection in circulating DNA is feasible and can be developed relatively soon.

### E. Conclusion

Early detection of ovarian cancer through regular screening can improve prognosis for cancer patients. Advances in biomarker development and better imaging techniques indicate that ovarian cancer can be accurately detected, although a definitive test has yet to emerge. In this study we evaluated the detection potential of methylation profiling using a panel of 56 potentially methylated genes. Profiles of tumor sections (n=30) of serous papillary adenocarcinoma were compared to profiles of uninvolved ovaries (n=30) from women of a high-risk group, and ten genes (BRCA1, EP300, NR3C1 (GR), MLH1, DNAJC15 (MCJ), CDKN1C (p57kip2), TP73, PGR (proximal promoter), PYCARD (TMS1), THBS1) emerged as components of a composite biomarker. In stratified five-fold cross-validation this biomarker identified ovarian cancer with 70% accuracy. Similar profiling of circulating DNA from blood of patients with serous papillary adenocarcinoma (n=33) and healthy controls (n=33), identified five genes (BRCA1, HIC1, PAX5, PGR (proximal promoter), THBS1) as components of the composite biomarker. This biomarker has 85% sensitivity and 61% specificity for detection of ovarian cancer as estimated by stratified five-fold cross-validation. Our results indicate that differential methylation profiling is possible with heterogeneous samples (whole sections of ovarian tissues and circulating DNA from blood). While the accuracy of developed biomarkers needs additional refinement, even at this time the blood-based biomarker can be useful as a first-line screening tool in combination with imaging techniques.

All publications and patents mentioned in the above specification are herein incorporated by reference. Various modifications and variations of the described compositions and methods of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the present invention.

1. A method for diagnosing cancer in a subject, comprising:
(a) reacting isolated genomic DNA from the subject and a methylation-sensitive restriction enzyme; wherein the genomic DNA comprises a plurality of promoters from different genes, and the enzyme cleaves unmethylated CpG sequences in the promoters and does not cleave methylated CpG sequences in the promoters;
(b) contacting the genomic DNA thus reacted and a plurality of pairs of specific primers in an amplification mixture, the pairs of specific primers being configured to hybridize to the genomic DNA and to amplify a plurality of different promoters through a region comprising an uncleaved CpG sequence;
(c) reacting the amplification mixture;
(d) detecting one or more amplified promoters in the reacted amplification mixture or the absence thereof, thereby diagnosing cancer in the subject selected from the group consisting of ovarian cancer, lung cancer, prostate cancer, pancreatic cancer, and colon cancer.

2. The method of claim 1, wherein the genomic DNA is isolated from blood.

3. The method of claim 1, wherein the genomic DNA is isolated from plasma.

4. The method of claim 1, wherein the genomic DNA is isolated from tissue of the subject.

5. The method of claim 1, wherein detecting one or more amplified promoters in the reacted amplification mixture or the absence thereof comprises:
(1) contacting a microarray and the reacted amplification mixture, the microarray comprising a plurality of DNA samples, each of which hybridizes to one of the plurality of different promoters; and
(2) detecting hybridization or the lack of hybridization between DNA in the reacted amplification mixture and one or more of the plurality of DNA samples of the microarray thereby obtaining a methylation profile.

6. The method of claim 5, further comprising comparing the methylation profile for the subject and a standard methylation profile selected from the group consisting of a standard methylation profile for non-cancerous samples, a standard methylation profile for cancerous samples, and both standard methylation profiles.

7. The method of claim 1, further comprising the step of separating the isolated genomic DNA of step (a) into: (i) a control sample and (ii) an experimental sample and adding control nucleic acid to both the control and experimental samples, wherein the control nucleic acid comprises at least one known CpG sequence that is unmethylated.

8. The method of claim 7, wherein the control sample is not reacted with the methylation-sensitive restriction enzyme and the experimental sample is reacted with the methylation-sensitive restriction enzyme, and wherein both the control and experimental samples are contacted with primers for the control nucleic acid under conditions such that a fragment of the control nucleic acid is amplified if the known CpG sequence is uncleaved.

9. The method of claim 1, wherein the plurality of pairs of specific primers comprises at least five pairs of specific primers.

10. The method of claim 9, wherein each of the five pairs of specific primers is configured to amplify a gene selected from the group consisting of FHIT, HMLH1, DNAJC15, MGMT, progesterone receptor, RARB, RPL15, PYCARD, and PLAU, and the diagnosed cancer is ovarian cancer.

11. The method of claim 9, wherein each of the five pairs of specific primers is configured to amplify a gene selected from the group consisting of BRCA1, EP300, NR3C1 (GR), MLH1, DNAJC15 (MCJ), CDKN1C (p57kip2), TP73, PGR (proximal promoter), THBS1, and PYCARD (TMS1), and the diagnosed cancer is ovarian cancer.

12. The method of claim 9, wherein each of the five pairs of specific primers is configured to amplify a gene selected from the group consisting of BRCA1, HIC1, PAX5, PGR (proximal promoter), and THBS1, and the diagnosed cancer is ovarian cancer.

13. The method of claim 9, wherein each of the five pairs of specific primers is configured to amplify a gene selected from the group consisting of CASP 8, CDKN1C, VHL, PAX5, DAPK1, NR3C1, MGMT, progesterone receptor, MLH1, RFC, TES, TNFSF11, CCND2, MYOD1, RB1, SFN, ESR1 promoter A, and GPC3, and the diagnosed cancer is lung cancer.

14. The method of claim 9, wherein each of the five pairs of specific primers is configured to amplify a gene selected from the group consisting of CASP 8, CDKN1C, VHL, PAX5, PGR (proximal promoter), and GPC3, and the diagnosed cancer is lung cancer.

15. The method of claim 9, wherein each of the five pairs of specific primers is configured to amplify a gene selected from the group consisting of BRCA1, CALCA, CASP 8, CCND2, EDNRB, EP 300, FHIT, GPC3, NR3C1, HIC, DNAJC15, FABP3, ABCB1, MSH2, CDKN1A, CDKN1C, PAX5, PGK1, PGR (distal promoter), S100A2, TES, THBS, and VHL, and the diagnosed cancer is prostate cancer.

16. The method of claim 9, wherein each of the five pairs of specific primers is configured to amplify a gene selected from the group consisting of SFN, BRCA1, DAPK1, EDNRB, NR3C1, DNAJC15, MUC2, CDKN1A, CDKN1C, PGK1, PGR, S100A2, TES, and VHL, and the diagnosed cancer is pancreatic cancer.

17. The method of claim 9, wherein each of the five pairs of specific primers is configured to amplify a gene selected from the group consisting of BRCA1, CASP 8, CCND2, DAPK1, ESR1, GPC3, NR3C1, ABCB1, MYOD1, CDKN1A, CDKN1C, PGK1, PGR, RARB, RB1, RFC, RPL15, S100A2, SOCS1, TES, THBS, and VHL, and the diagnosed cancer is colon cancer.

18. The method of claim 1, wherein the amplification mixture is a multiplex amplification mixture.

19. A method for diagnosing pancreatic cancer in a subject, comprising:
(a) reacting a plasma sample from the subject and reagents for detecting methylation status of genomic DNA in the sample;
(b) determining the methylation status for a plurality of genes to generate a methylation profile, thereby diagnosing pancreatic cancer in the subject.

20. A method for diagnosing colon cancer in a subject, comprising:
(a) reacting a plasma sample from the subject and reagents for detecting methylation status of genomic DNA in the sample;
(b) determining the methylation status for a plurality of genes to generate a methylation profile, thereby diagnosing colon cancer in the subject.


SEQUENCE LISTING (223 sequences)

SEQ ID NO: 1 - DNA, 21 bp, Homo sapiens
tgggaaatgtgtccaacaaac

SEQ ID NO: 2 - DNA, 21 bp, Homo sapiens
gccaccaattccctgaaactc

SEQ ID NO: 3 - DNA, 17 bp, Homo sapiens
aatcgcgtgcgccgttc

SEQ ID NO: 4 - DNA, 20 bp, Homo sapiens
atcggcaaaggcgaggctct

SEQ ID NO: 5 - DNA, 20 bp, Homo sapiens
gcgccttccactgcgatatt

SEQ ID NO: 6 - DNA, 21 bp, Homo sapiens
gttcccaccaatgccggactc

SEQ ID NO: 7 - DNA, 19 bp, Homo sapiens
ctgagaggctgctgcttag

SEQ ID NO: 8 - DNA, 21 bp, Homo sapiens
gaatacccatctgtcagcttc

SEQ ID NO: 9 - DNA, 23 bp, Homo sapiens
tgcggagagcgagtcttagatac

SEQ ID NO: 10 - DNA, 21 bp, Homo sapiens
ccaattacgcgtgacctcaac

SEQ ID NO: 11 - DNA, 18 bp, Homo sapiens
cggctggtgagcaggaag

SEQ ID NO: 12 - DNA, 25 bp, Homo sapiens
gcatctgagctccaagtccactctg

SEQ ID NO: 13 - DNA, 19 bp, Homo sapiens
gaccgtgctggcggacttc

SEQ ID NO: 14 - DNA, 20 bp, Homo sapiens
tggccacaccgatgcagctt

SEQ ID NO: 15 - DNA, 18 bp, Homo sapiens
aggatctggagcgaactg

SEQ ID NO: 16 - DNA, 17 bp, Homo sapiens
ggctccggaagtgactg

SEQ ID NO: 17 - DNA, 20 bp, Homo sapiens
ctccagcttgggtgaaagag

SEQ ID NO: 18 - DNA, 20 bp, Homo sapiens
cgtaccgctgattggctgag

SEQ ID NO: 19 - DNA, 20 bp, Homo sapiens
gagagggcatcaggaaggag

SEQ ID NO: 20 - DNA, 20 bp, Homo sapiens
aggccgcaggcaagaaccag

SEQ ID NO: 21 - DNA, 21 bp, Homo sapiens
aggaggtgagtgtctcttgtc

SEQ ID NO: 22 - DNA, 21 bp, Homo sapiens
ctggagagggatgcggactcg

SEQ ID NO: 23 - DNA, 19 bp, Homo sapiens
ggtgccctactacctggag

SEQ ID NO: 24 - DNA, 18 bp, Homo sapiens
ccggcgagagaacttgac

SEQ ID NO: 25 - DNA, 19 bp, Homo sapiens
ctctggctgtgccacactg

SEQ ID NO: 26 - DNA, 21 bp, Homo sapiens
gcacaaagaatcctacaagtc

SEQ ID NO: 27 - DNA, 20 bp, Homo sapiens
aatgcccatttgtgcaacga

SEQ ID NO: 28 - DNA, 18 bp, Homo sapiens
cgtactgagcgggtccac

SEQ ID NO: 29 - DNA, 21 bp, Homo sapiens
gtgcggtacagcctttcgtta

SEQ ID NO: 30 - DNA, 19 bp, Homo sapiens
tcctgtgaccggacagagc

SEQ ID NO: 31 - DNA, 21 bp, Homo sapiens
agtggccctgaggagcaagag

SEQ ID NO: 32 - DNA, 20 bp, Homo sapiens
ccagagcgccctgtgtagag

SEQ ID NO: 33 - DNA, 21 bp, Homo sapiens
gcgtcaccaacaggttgcatc

SEQ ID NO: 34 - DNA, 20 bp, Homo sapiens
tctccttccacccacagaat

SEQ ID NO: 35 - DNA, 18 bp, Homo sapiens
tccgggatcgcagcggtc

SEQ ID NO: 36 - DNA, 20 bp, Homo sapiens
cgaagactgcggcggcgaaa

SEQ ID NO: 37 - DNA, 22 bp, Homo sapiens
gtaaagttctccgccctgaatg

SEQ ID NO: 38 - DNA, 18 bp, Homo sapiens
ccggaccaggagaaggag

SEQ ID NO: 39 - DNA, 20 bp, Homo sapiens
acgttgccacggtctgggat

SEQ ID NO: 40 - DNA, 19 bp, Homo sapiens
caggcaggcccggcctttg

SEQ ID NO: 41 - DNA, 19 bp, Homo sapiens
cgccacataccgctcgtag

SEQ ID NO: 42 - DNA, 20 bp, Homo sapiens
gctgtccgctcttcctattg

SEQ ID NO: 43 - DNA, 20 bp, Homo sapiens
cttagcgcggtgtagaccgt

SEQ ID NO: 44 - DNA, 19 bp, Homo sapiens
gagccatagcgaggctgag

SEQ ID NO: 45 - DNA, 19 bp, Homo sapiens
catggctgcccgtggtgtc

SEQ ID NO: 46 - DNA, 18 bp, Homo sapiens
ggcgtcaaagcccagcac

SEQ ID NO: 47 - DNA, 21 bp, Homo sapiens
aagtcccgccctttcagctac

SEQ ID NO: 48 - DNA, 20 bp, Homo sapiens
atagggaagggcccggaatg

SEQ ID NO: 49 - DNA, 21 bp, Homo sapiens
gccaccaggcagtgagagtga

SEQ ID NO: 50 - DNA, 22 bp, Homo sapiens
ggcctctaggcactctggaatc

SEQ ID NO: 51 - DNA, 20 bp, Homo sapiens
tccactaaagtcggagtatc

SEQ ID NO: 52 - DNA, 17 bp, Homo sapiens
tggtccagtgccactac

SEQ ID NO: 53 - DNA, 18 bp, Homo sapiens
acgggccatttggcaaac

SEQ ID NO: 54 - DNA, 18 bp, Homo sapiens
gtcggcgcatgcccagtg

SEQ ID NO: 55 - DNA, 19 bp, Homo sapiens
cttccgggcacattacgag

SEQ ID NO: 56 - DNA, 20 bp, Homo sapiens
cacacccactaagctgtttc

SEQ ID NO: 57 - DNA, 18 bp, Homo sapiens
cagggctgcctcatcctg

SEQ ID NO: 58 - DNA, 17 bp, Homo sapiens
ctcccagacgcgacttg

SEQ ID NO: 59 - DNA, 21 bp, Homo sapiens
gttgttgcactcgtgcgtttc

SEQ ID NO: 60 - DNA, 19 bp, Homo sapiens
cggcacgccctttccaaac

SEQ ID NO: 61 - DNA, 19 bp, Homo sapiens
ctggcctcccggcgatcac

SEQ ID NO: 62 - DNA, 22 bp, Homo sapiens
cattaccctcccgtcgtccttc

SEQ ID NO: 63 - DNA, 21 bp, Homo sapiens
agcatggagccttcggctgac

SEQ ID NO: 64 - DNA, 21 bp, Homo sapiens
tccggagaatcgaagcgctac

SEQ ID NO: 65 - DNA, 20 bp, Homo sapiens
tggagagtgccaactcattc

SEQ ID NO: 66 - DNA, 20 bp, Homo sapiens
tcagcgcggccctgatatac

SEQ ID NO: 67 - DNA, 18 bp, Homo sapiens
ctccgaggccagccagag

SEQ ID NO: 68 - DNA, 22 bp, Homo sapiens
ggtggaagggaggctgacgaag

SEQ ID NO: 69 - DNA, 18 bp, Homo sapiens
atcgccgtggtgttgttg

SEQ ID NO: 70 - DNA, 19 bp, Homo sapiens
ctgtccggtggtggactct

SEQ ID NO: 71 - DNA, 18 bp, Homo sapiens
aaaggcggcgggaaggag

SEQ ID NO: 72 - DNA, 18 bp, Homo sapiens
cggcccctaggcgggtta

SEQ ID NO: 73 - DNA, 18 bp, Homo sapiens
aaacccggcctgcgctcg

SEQ ID NO: 74 - DNA, 18 bp, Homo sapiens
ctagccagcgcacctacg

SEQ ID NO: 75 - DNA, 21 bp, Homo sapiens
ctaagtcgggaaggttccttg

SEQ ID NO: 76 - DNA, 20 bp, Homo sapiens
gcttgcagaatgcggaacac

SEQ ID NO: 77 - DNA, 20 bp, Homo sapiens
tcggccatacctatctccct

SEQ ID NO: 78 - DNA, 19 bp, Homo sapiens
agccggtggatcttcggga

SEQ ID NO: 79 - DNA, 20 bp, Homo sapiens
agtactctgcgtctccagtc

SEQ ID NO: 80 - DNA, 18 bp, Homo sapiens
cagagggaggagaaagtg

SEQ ID NO: 81 - DNA, 18 bp, Homo sapiens
gtttagggcttgcatgtg

SEQ ID NO: 82 - DNA, 18 bp, Homo sapiens
caccaactcccaggattc

SEQ ID NO: 83 - DNA, 20 bp, Homo sapiens
cgcggctctcctcagctcct

SEQ ID NO: 84 - DNA, 20 bp, Homo sapiens
cccagatgaagtcgccacag

SEQ ID NO: 85 - DNA, 21 bp, Homo sapiens
ccacagtcacccaccagactc

SEQ ID NO: 86 - DNA, 20 bp, Homo sapiens
tcctctcccgactcccgtta

SEQ ID NO: 87 - DNA, 22 bp, Homo sapiens
gatccagcttgcgccaggaatg

SEQ ID NO: 88 - DNA, 17 bp, Homo sapiens
cgtcccgcgaacgcgtc

SEQ ID NO: 89 - DNA, 20 bp, Homo sapiens
ctagggtgcggtcggacttg

SEQ ID NO: 90 - DNA, 19 bp, Homo sapiens
gccgccatcttgactccag

SEQ ID NO: 91 - DNA, 21 bp, Homo sapiens
gcggtgcgtgaaacaaacctg

SEQ ID NO: 92 - DNA, 24 bp, Homo sapiens
cccagagcgtcatgggacatgtag

SEQ ID NO: 93 - DNA, 22 bp, Homo sapiens
gggttggatttcagcaggatag

SEQ ID NO: 94 - DNA, 22 bp, Homo sapiens
cagggaagggaacaccacatac

SEQ ID NO: 95 - DNA, 21 bp, Homo sapiens
cacctgtgcctgctagaagag

SEQ ID NO: 96 - DNA, 22 bp, Homo sapiens
cctgcgccagtcttttaaaccg

SEQ ID NO: 97 - DNA, 19 bp, Homo sapiens
ttgccgtgccaacacagtc

SEQ ID NO: 98 - DNA, 22 bp, Homo sapiens
cttgaaagcgtttcgccttccg

SEQ ID NO: 99 - DNA, 19 bp, Homo sapiens
cgggcgcgttaaggaagtt

SEQ ID NO: 100 - DNA, 22 bp, Homo sapiens
cccgtaacctcctctccttacc

SEQ ID NO: 101 - DNA, 20 bp, Homo sapiens
aaacgggcccagtctctagt

SEQ ID NO: 102 - DNA, 20 bp, Homo sapiens
cgcgcaactttccagctaga

SEQ ID NO: 103 - DNA, 20 bp, Homo sapiens
acgcccagagaatcccttcg

SEQ ID NO: 104 - DNA, 20 bp, Homo sapiens
gcgccgctcaacagccactc

SEQ ID NO: 105 - DNA, 20 bp, Homo sapiens
tggaattgagggagcttcac

SEQ ID NO: 106 - DNA, 20 bp, Homo sapiens
aaggcgcttccttactacac

SEQ ID NO: 107 - DNA, 23 bp, Homo sapiens
ctcttggacctccagaaagacag

SEQ ID NO: 108 - DNA, 18 bp, Homo sapiens
cttggagcccggctttgg

SEQ ID NO: 109 - DNA, 24 bp, Homo sapiens
ttctgtctgtgcttcttgggagag

SEQ ID NO: 110 - DNA, 23 bp, Homo sapiens
ccgcaacgctcacaaagatttgg

SEQ ID NO: 111 - DNA, 20 bp, Homo sapiens
ctatttccgcgagcgcgttc

SEQ ID NO: 112 - DNA, 20 bp, Homo sapiens
attccctccgcgatccagac

SEQ ID NO: 113 - DNA, 29 bp, Homo sapiens
ggcctctgacctatgagctccagactgtg

SEQ ID NO: 114 - DNA, 23 bp, Homo sapiens
aatcgcgtgcgccgttccgaaag

SEQ ID NO: 115 - DNA, 23 bp, Homo sapiens
atcggcaaaggcgaggctctgtg

SEQ ID NO: 116 - DNA, 24 bp, Homo sapiens
gcgccttccactgcgatattgctc

SEQ ID NO: 117 - DNA, 22 bp, Homo sapiens
gttcccaccaatgccggactcg

SEQ ID NO: 118 - DNA, 25 bp, Homo sapiens
ctgagaggctgctgcttagcggtag

SEQ ID NO: 119 - DNA, 28 bp, Homo sapiens
gaatacccatctgtcagcttcggaaatc

SEQ ID NO: 120 - DNA, 27 bp, Homo sapiens
tgcggagagcgagtcttagatacccag

SEQ ID NO: 121 - DNA, 26 bp, Homo sapiens
ccaattacgcgtgacctcaacagctc

SEQ ID NO: 122 - DNA, 23 bp, Homo sapiens
ccgctgggaggctgccaaagttc

SEQ ID NO: 123 - DNA, 28 bp, Homo sapiens
gcatctgagctccaagtccactctgttc

SEQ ID NO: 124 - DNA, 22 bp, Homo sapiens
gaccgtgctggcggacttcacc

SEQ ID NO: 125 - DNA, 24 bp, Homo sapiens
tggccacaccgatgcagctttcta

SEQ ID NO: 126 - DNA, 24 bp, Homo sapiens
ggagagggagtcgccaggaatgtg

SEQ ID NO: 127 - DNA, 24 bp, Homo sapiens
cagggacgccgcggaagaatgaag

SEQ ID NO: 128 - DNA, 26 bp, Homo sapiens
ctccagcttgggtgaaagagtgagac

SEQ ID NO: 129 - DNA, 25 bp, Homo sapiens
cgtaccgctgattggctgagggttc

SEQ ID NO: 130 - DNA, 27 bp, Homo sapiens
gagagggcatcaggaaggagtttcgac

SEQ ID NO: 131 - DNA, 24 bp, Homo sapiens
gcaggcaagaaccagcgcaaccag

SEQ ID NO: 132 - DNA, 24 bp, Homo sapiens
tctcttgtcgcctcctcctctccc

SEQ ID NO: 133 - DNA, 25 bp, Homo sapiens
ctggagagggatgcggactcgatag

SEQ ID NO: 134 - DNA, 25 bp, Homo sapiens
ggtgccctactacctggagaacgag

SEQ ID NO: 135 - DNA, 25 bp, Homo sapiens
ccggcgagagaacttgactctgaac

SEQ ID NO: 136 - DNA, 24 bp, Homo sapiens
ccacactgctccctgtgagcagac

SEQ ID NO: 137 - DNA, 25 bp, Homo sapiens
cccatggagaacagcaatcctcatc

SEQ ID NO: 138 - DNA, 23 bp, Homo sapiens
aatgcccatttgtgcaacgaacc

SEQ ID NO: 139 - DNA, 22 bp, Homo sapiens
cgtactgagcgggtccaccaac

SEQ ID NO: 140 - DNA, 24 bp, Homo sapiens
gtgcggtacagcctttcgttacac

SEQ ID NO: 141 - DNA, 24 bp, Homo sapiens
tcctgtgaccggacagagcagagc

SEQ ID NO: 142 - DNA, 24 bp, Homo sapiens
agtggccctgaggagcaagagacg

SEQ ID NO: 143 - DNA, 24 bp, Homo sapiens
caccctcctctcgcactgccttcg

SEQ ID NO: 144 - DNA, 25 bp, Homo sapiens
gcgtcaccaacaggttgcatcgttc

SEQ ID NO: 145 - DNA, 22 bp, Homo sapiens
tctccttccacccacagaatcc

SEQ ID NO: 146 - DNA, 22 bp, Homo sapiens
tctccttccacccacagaatcc

SEQ ID NO: 147 - DNA, 23 bp, Homo sapiens
cgaagactgcggcggcgaaactc

SEQ ID NO: 148 - DNA, 25 bp, Homo sapiens
ggtaaagttctccgccctgaatgac

SEQ ID NO: 149 - DNA, 27 bp, Homo sapiens
ggaccaggagaaggagcaggaggtgag

SEQ ID NO: 150 - DNA, 25 bp, Homo sapiens
acgttgccacggtctgggatcagag

SEQ ID NO: 151 - DNA, 23 bp, Homo sapiens
caggcaggcccggcctttgtctc

SEQ ID NO: 152 - DNA, 25 bp, Homo sapiens
cgccacataccgctcgtagtattcg

SEQ ID NO: 153 - DNA, 28 bp, Homo sapiens
gctgtccgctcttcctattggttcgttt

SEQ ID NO: 154 - DNA, 24 bp, Homo sapiens
cttagcgcggtgtagaccgtgatt

SEQ ID NO: 155 - DNA, 23 bp, Homo sapiens
gagccatagcgaggctgaggttg

SEQ ID NO: 156 - DNA, 23 bp, Homo sapiens
catggctgcccgtggtgtcatcg

SEQ ID NO: 157 - DNA, 23 bp, Homo sapiens
ggcgtcaaagcccagcacaaagc

SEQ ID NO: 158 - DNA, 24 bp, Homo sapiens
aagtcccgccctttcagctacctc

SEQ ID NO: 159 - DNA, 26 bp, Homo sapiens
atagggaagggcccggaatgggaaag

SEQ ID NO: 160 - DNA, 24 bp, Homo sapiens
gccaccaggcagtgagagtgaagg

SEQ ID NO: 161 - DNA, 25 bp, Homo sapiens
tggcctctaggcactctggaatctg

SEQ ID NO: 162 - DNA, 23 bp, Homo sapiens
tttcacgtcttggtggccgttcc

SEQ ID NO: 163 - DNA, 23 bp, Homo sapiens
tggtccagtgccactacggtttg

SEQ ID NO: 164 - DNA, 23 bp, Homo sapiens
acgggccatttggcaaactaagg

SEQ ID NO: 165 - DNA, 22 bp, Homo sapiens
ggcctgaggcagtctgcgcatc

SEQ ID NO: 166 - DNA, 26 bp, Homo sapiens
cctggtggcaacctacccttgcatac

SEQ ID NO: 167 - DNA, 25 bp, Homo sapiens
agtcagcttccagggctgcgtttcg

SEQ ID NO: 168 - DNA, 24 bp, Homo sapiens
cagggctgcctcatcctgaagaag

SEQ ID NO: 169 - DNA, 24 bp, Homo sapiens
ccaaagacagggccaggcacacag

SEQ ID NO: 170 - DNA, 25 bp, Homo sapiens
gttgttgcactcgtgcgtttctctg

SEQ ID NO: 171 - DNA, 24 bp, Homo sapiens
cggcacgccctttccaaacctctc

SEQ ID NO: 172 - DNA, 24 bp, Homo sapiens
acggaattctttgccggctggctc

SEQ ID NO: 173 - DNA, 25 bp, Homo sapiens
cattaccctcccgtcgtccttctgc

SEQ ID NO: 174 - DNA, 24 bp, Homo sapiens
agcatggagccttcggctgactgg

SEQ ID NO: 175 - DNA, 28 bp, Homo sapiens
tccggagaatcgaagcgctacctgattc

SEQ ID NO: 176 - DNA, 24 bp, Homo sapiens
gggaaatgtgtccagcgcaccaac

SEQ ID NO: 177 - DNA, 24 bp, Homo sapiens
tcagcgcggccctgatatacaacc

SEQ ID NO: 178 - DNA, 26 bp, Homo sapiens
ctccgaggccagccagagcaggtttg

SEQ ID NO: 179 - DNA, 25 bp, Homo sapiens
ggtggaagggaggctgacgaagaag

SEQ ID NO: 180 - DNA, 27 bp, Homo sapiens
atcgccgtggtgttgttgaaactgaaa

SEQ ID NO: 181 - DNA, 27 bp, Homo sapiens
ggtggtggactcttctgcgtcgggttc

SEQ ID NO: 182 - DNA, 21 bp, Homo sapiens
gagcgccgggaggagaccttg

SEQ ID NO: 183 - DNA, 23 bp, Homo sapiens
cggcccctaggcgggttatatgg

SEQ ID NO: 184 - DNA, 24 bp, Homo sapiens
aaacccggcctgcgctcgtctaag

SEQ ID NO: 185 - DNA, 23 bp, Homo sapiens
ctagccagcgcacctacgggaag

SEQ ID NO: 186 - DNA, 28 bp, Homo sapiens
ctaagtcgggaaggttccttgcggttcg

SEQ ID NO: 187 - DNA, 25 bp, Homo sapiens
cgggcaggaacagggcccacactac

SEQ ID NO: 188 - DNA, 25 bp, Homo sapiens
tcggccatacctatctccctggacg

SEQ ID NO: 189 - DNA, 25 bp, Homo sapiens
agccggtggatcttcgggaagttcg

SEQ ID NO: 190 - DNA, 25 bp, Homo sapiens
tgcgtctccagtcctcggacagaag

SEQ ID NO: 191 - DNA, 26 bp, Homo sapiens
cctgcccttggcctccatcctgtcgt

SEQ ID NO: 192 - DNA, 23 bp, Homo sapiens
acagacagaaaggcgcacagagg

SEQ ID NO: 193 - DNA, 24 bp, Homo sapiens
caccaactcccaggattctcacag

SEQ ID NO: 194 - DNA, 22 bp, Homo sapiens
cgcggctctcctcagctccttc

SEQ ID NO: 195 - DNA, 25 bp, Homo sapiens
cccagatgaagtcgccacagaggtc

SEQ ID NO: 196 - DNA, 25 bp, Homo sapiens
ccacagtcacccaccagactctttg

SEQ ID NO: 197 - DNA, 25 bp, Homo sapiens
tcctctcccgactcccgttacaaaa

SEQ ID NO: 198 - DNA, 25 bp, Homo sapiens
gatccagcttgcgccaggaatgcag

SEQ ID NO: 199 - DNA, 20 bp, Homo sapiens
gtcccgcgaacgcgtcctga

SEQ ID NO: 200 - DNA, 22 bp, Homo sapiens
ctagggtgcggtcggacttgcc

SEQ ID NO: 201 - DNA, 25 bp, Homo sapiens
gccgccatcttgactccagtcggaa

SEQ ID NO: 202 - DNA, 26 bp, Homo sapiens
gcggtgcgtgaaacaaacctgttctc

SEQ ID NO: 203 - DNA, 27 bp, Homo sapiens
cccagagcgtcatgggacatgtagttc

SEQ ID NO: 204 - DNA, 25 bp, Homo sapiens
ggcatgggcatgtgtgggcacgttc

SEQ ID NO: 205 - DNA, 27 bp, Homo sapiens
ccacataccagggcctgtgggcagttg

SEQ ID NO: 206 - DNA, 28 bp, Homo sapiens
cacctgtgcctgctagaagagtctcatc

SEQ ID NO: 207 - DNA, 26 bp, Homo sapiens
cctgcgccagtcttttaaaccggctc

SEQ ID NO: 208 - DNA, 24 bp, Homo sapiens
ttgccgtgccaacacagtctctgc

SEQ ID NO: 209 - DNA, 27 bp, Homo sapiens
cttgaaagcgtttcgccttccgctgtc

SEQ ID NO: 210 - DNA, 24 bp, Homo sapiens
cgggcgcgttaaggaagttgccca

SEQ ID NO: 211 - DNA, 26 bp, Homo sapiens
cccgtaacctcctctccttaccagaa

SEQ ID NO: 212 - DNA, 26 bp, Homo sapiens
aaacgggcccagtctctagtatccac

SEQ ID NO: 213 - DNA, 26 bp, Homo sapiens
gcgcgcaactttccagctagaaagtg

SEQ ID NO: 214 - DNA, 23 bp, Homo sapiens
acgcccagagaatcccttcggag

SEQ ID NO: 215 - DNA, 23 bp, Homo sapiens
cgaacacgggaaacctgcggaac

SEQ ID NO: 216 - DNA, 27 bp, Homo sapiens
tggaattgagggagcttcacgcttcta

SEQ ID NO: 217 - DNA, 28 bp, Homo sapiens
aaggcgcttccttactacacccttggtc

SEQ ID NO: 218 - DNA, 27 bp, Homo sapiens
ggacctccagaaagacagctgaggatg

SEQ ID NO: 219 - DNA, 24 bp, Homo sapiens
cttggagcccggctttgggtcctg

SEQ ID NO: 220 - DNA, 27 bp, Homo sapiens
gtcgcgtgatgaagacttcacagctcc

SEQ ID NO: 221 - DNA, 26 bp, Homo sapiens
cccaacagcgtctggactgaggaatc

SEQ ID NO: 222 - DNA, 24 bp, Homo sapiens
ctatttccgcgagcgcgttccatc

SEQ ID NO: 223 - DNA, 24 bp, Homo sapiens
attccctccgcgatccagaccacc